(19) United States
(12) Patent Application Publication
(10) Pub. No.: US 2004/0076954 A1
(43) Pub. Date: Apr. 22, 2004

(54) Genomics-driven high speed cellular assays, development thereof, and collections of cellular reporters

(75) Inventors: Jeremy S. Caldwell, Cardiff, CA (US); John B. Hogenesch, Encinitas, CA (US); Andrew I. Su, La Jolla, CA (US)

Correspondence Address: HELLER EHRMAN WHITE & MCAULIFFE LLP, 4350 LA JOLLA VILLAGE DRIVE, 7TH FLOOR, SAN DIEGO, CA, 92122-1246, US

(73) Assignee: IRM, LLC

(21) Appl. No.: 10/097,034

(22) Filed: Mar. 12, 2002

Related U.S. Application Data
(60) Provisional application No. 60/275,148, filed on Mar. 12, 2001.
(60) Provisional application No. 60/274,979, filed on Mar. 12, 2001.
(60) Provisional application No. 60/275,070, filed on Mar. 12, 2001.

(51) Int. Cl. C12Q 1/68; G01N 33/53; G01N 33/567; C12N 5/06; C12N 15/85

(52) U.S. Cl. 435/6; 435/7.2; 435/455; 435/325

(57) ABSTRACT

Methods for identifying responder genes and regulatory regions that confer responsiveness to a test substance or other perturbation are provided. Regulatory regions identified by such methods or other methods are cloned into expression constructs to control expression of a nucleic acid molecule that encodes, for example, a selectable marker or reporter, and introduced into cells. The resulting cells are used, for example, in high throughput screening assays for profiling substances and conditions and for studying the function of the regulatory region mediating the response. Addressable collections of the cells are also provided.

### RELATED APPLICATIONS

Benefit of priority under 35 U.S.C. §119(e) is claimed to the following applications: U.S. provisional application Ser. No. 60/275,148, filed Mar. 12, 2001, by Jeremy S. Caldwell, entitled, “Chemical and Combinatorial Biology Strategies for High-Throughput Gene Functionalization;” U.S. provisional application Ser. No. 60/274,979, filed Mar. 12, 2001, by Jeremy S. Caldwell, entitled, “Cellular Reporter Arrays;” and U.S. provisional application Ser. No. 60/275,070, filed Mar. 12, 2001, by Andrew Su, John B. Hogenesch, Sumit Chanda and Jeremy S. Caldwell, entitled, “Genomics-driven high speed cellular assay development.” This application is related to U.S. provisional application Ser. No. 60/275,266, filed Mar. 12, 2001, by Jeremy S. Caldwell, entitled, “Identification of cellular targets for biologically active molecules”. The subject matter of each application is herein incorporated by reference in its entirety.

### FIELD OF INVENTION

Fully automated systems and methods for screening cells are provided. Methods for identifying gene regulatory regions and producing gene regulatory region libraries are provided. In particular, arrays of cells with regulatory regions responsive to a stimulus for assessing the effects of agents are provided. The cellular arrays serve as biosensors for assessing effects of any agent, including small molecules and other signals.

### BACKGROUND

A power of cell-based screening is the ability to blindly interrogate complex cellular pathways to assess critical components and to identify small molecule effectors. The process, however, often is stymied because there are inadequate methods to determine the cellular targets of a small molecule effector found in a screen. Screening assays, thus, are generally black boxes. A cell is contacted or exposed to an effector molecule or condition, and an effect is observed. It, however, is not possible to identify with what a test compound or test condition is reacting or affecting in the cell. Many drug development campaigns are thwarted by the lack of target information; structure activity relationship studies are impossible, and appropriate animal model tests and eventually phase I-III clinical trials can be hampered without target identification.

Thus, there is a need for improved cell-based assays and the development of ways to obtain target information. Therefore, among the objects herein, it is an object to provide improved cell-based assays and high throughput assays and to provide methods for obtaining target information.

### SUMMARY

Collections of reporter cells, which serve as real-time, cell-based alternative to DNA microarrays, are provided. The cells are produced by introducing nucleic acid elements that include regulatory elements for all genes or a subset of genes in a genome, tissue, cell, organism or other selected target into reporter gene cassettes, which are then introduced stably or transiently into cells to produce the collections. The cells are provided as addressable collections, such as in high-density microtiter plates or other addressable format, in loci on the plate or other format. Each contains a cellular population expressing a unique reporter gene construct. The collections of cells have a variety of uses, including, but are not limited to, drug target identification and drug discovery.

In particular, collections of reporter cells for use in screening methods, including high throughput methods of screening that are automated or partially automated, are provided. The collections of cells serve as biosensors to assess the effects of any perturbation, such a an external or internal condition, on the cells from which the regulatory regions in the reporter gene constructs are derived can be inferred. The collections also provide a means to obtain target information when screened with known and test compounds or other conditions. The collections optionally include control cells that, for example, do not contain a regulatory region linked to a reporter or they do not contain a reporter.

Cell-based assays and high throughput cell-based assays that employ the collections are provided. A collection of cells is exposed to a perturbations, such as treatment with characterized and/or unchacterized cell modulators or conditions whose effects are monitored. Such perturbations, include, but are not limited to, nucleic acid expression vectors, nucleic acids, oligonucleotides, proteins, peptides, antibodies, small molecules, extracts, mixtures of samples, or multivariate combinations of these inputs, changes in pH, temperature, oxygen pressure, external medium, different time periods and other conditions. The effect of these inputs on cellular reporter activity is measured using any suitable device or means, such as standard plate readers, charge coupled devices (CCDs) and video monitors or even visually observed.

The patterns of changes in cellular reporter activity affected by these inputs generates constitute a unique fingerprint for each characterized pertubation, such as a condition. Profiles of characterized perturbations can be determined and stored, such as in a database. By comparing profiles of unknown cell perturbations with the profiles from characterized perturbations, functions are ascribed to uncharacterized perturbations. Similarly, perturbations with similar patterns can be clustered or group to aid in selecting candidates for further study or to identify heretofore unknown relationships.

Also provided are methods for obtaining target information. By knowing what regulatory regions are activated, the collections can be used to identify cellular targets in a particular pathway.

Also provided are methods for producing the collections of reporter cells, particularly addressable collections, of such cells. The collections of cells, which contain regulatory regions linked to nucleic acids encoding reporters or nucleic acid reporters, are produced by identifying and isolating collections of promoter and regulatory regions from a desired target organism or tissue type or other sub-genomic fraction and introducing the identified regulatory regions operatively linked to reporters into cells to produce a collection of cells that are substantially identical, except that each set of cells contains a different regulatory and/or promoter region.

The methods herein provide rapid selection of gene regulatory regions appropriate for robust high-throughput screening assays and production of reporters whose expression is regulated by the regulatory regions and living cells that respond to the substance or stimulus.

Methods for identifying responder genes and regulatory regions that confer responsiveness to a perturbatoins, such as a test substance or other condition. for use in the reporter gene constructs and for introduction into cells are provided.

Thus, also provided are screening assays for identifying the cis acting gene regulatory regions, such as regions of genes that contain promoters and/or other regulatory sequences, such as enhancers, silencers, transcription factor binding sites, enhancers, scaffold attachment regions. The resulting regions and genes can be introduced into vectors and used to express heterologous proteins under the original perturbation, such as a condition, including but are not limited to, small effector molecules.

The regulatory/promoter regions can be identified and isolated by any suitable method. First, for example, using high-throughput screening methods, such as an oligonucleotide array, a gene expression profile of a cell, tissue or organ, or a biological sample from a subject, is obtained in the presence and absence of a perturbation, such as a test substance or a modulator. Next the regulatory regions are obtained. For example, one such method includes the steps of: (a) identifying protein-encoding sequences in an organism or tissue, such as from a database of DNA sequences of the organism or tissue; (b) designing primers for amplifying untranslated sequences that contain transcriptional regulatory sequences, including promoters, which are typically upstream of the protein encoding sequences in genomic DNA; (c) amplifying the untranslated sequences using the primers, thereby obtaining nucleic acid molecules that include regulatory regions, such as promoters.

The resulting promoters are then linked to nucleic acid encoding a reporter and a method for producing the cells can further include: (d) producing a plurality of reporter constructs that each contain one of the promoters operably linked to nucleic acid encoding a reporter, such as a detectable marker; and (d) introducing the reporter constructs into cells to produce a collections of reporter cell that each contain a reporter construct. The resulting cells can be introduced or produced as addressable arrays, such as microtiter plates with wells or surfaces for attaching the cells, or other solid surfaces that can be addressably encoded.

Responder genes, particularly those herein designated as robust responders, whose expression is increased or decreased a predetermined amount, typically at least 0.5-fold to 10-fold, generally at least two to three-fold, in response to the substance or stimulus, are identified and candidate gene regulatory regions, including promoters are selected using genomic sequence data or methods that permit or provide for such identification. Reporter gene constructs driven by the gene regulatory regions are produced and introduced into cells thereby producing cells containing the reporters, designated responder cells herein, that respond to the substance or stimulus or other perturbation. A plurality, such as a library, of the resulting responder cells are provided. Each cell contains a reporter driven by a different gene regulatory region. Such cells can be provided in addressable arrays, such as positionally addressable or labeled or identified in other ways. There resulting arrays are used in high-throughput screening assays for expression profiling of test substances or stimuli or other modulators of gene or gene expression activity.

For example, the reporter cells can be produced in a two-dimensional array or panel, for examples in wells of a microtiter plate Such arrays can include a large number of reporter cells, for example 96 or higher multiples thereof (i.e. 96×2, 96×3, 96×4 . . . 96×n, where n is 1 to any desired number, typically 15-20) or more different reporter cells, each representing a different promoter. Automated screening methods employing the addressable arrays are also provided herein.

The assays can be used to identify regulatory regions from any organism or tissue or organ or other subset of all regulatory regions. The regulatory regions can be selected to be those that are most responsive or are responsive when cells containing them are exposed to particular perturbations or sets thereof. Regulatory regions identified by such methods or other methods are cloned into expression constructs to control expression of a nucleic acid molecule that encodes, for example, a reporter, such as a detectable marker, and introduced into cells. The resulting collections cells are used, for example, in the high throughput screening assays for profiling perturbations, such as substances and conditions, and for studying the function of the regulatory region mediating the response.

Vectors that can infect a broad spectrum of cell types for expression reporter gene constructs in which reporter expression is modulated by the regulatory region are also provided. Also provided are cell specific vectors for expression of reporter gene constructs designed for expression in the specific cell types. In one embodiment, retroviral vectors that are designed for use in the processes are provided herein. These vectors deliver high-titer retroviral production, and ubiquitous and high-level gene expression in target cells. The vectors are optimized to facilitate image-based cDNA matrix-based expression screening. In particular retroviral vectors containing a unidirectional transcriptional blocker; a scaffold attachment region; and a robust responder regulatory region operatively linked to nucleic acid encoding a reporter gene are provided. These vectors can be designed to be self-inactivating. Any suitable retrovirus may be employed used. In one particular embodiment, an LTR is from a moloney murine leukemia virus (MoMLV).

The resulting addressable collections of cells serve as biosensors for assessing the effects of perturbatoins, such as conditions, including extracellular signals, thereon. Hence, methods for assessing the effect(s) of a perturbation, such as a small molecule on a cell are provided. In practicing such methods, reporter cells, such as the addressable arrays of such cells provided herein, are contacted with one or a plurality of test or known molecules or other perturbation. For any perturbation, the results for a particular array can serve as a fingerprint of the effects. Hence for any given signal, certain cells will respond or have altered responses compared to a control cell, such as a cell that does not have a reporter construct. The regulatory region/promoter in each responding cell is known. Sets of responding regions serve as a fingerprint of the perturbation. In addition, it is possible to deduce pathways based upon the effects. For example, if all one knows is that a test compound, such as a TNF antagonist, has a particular activity it is possible to identify where in a pathway it acts. To do each promoter in the pathway is separately over-expressed in the presence (and absence) of the inhibitor. If the inhibitor no longer inhibits when it a particular promoter is overexpressed, then that must be the target of the inhibitor.

Collections of responder regions and cells can be prepared for any desired perturbatoin or input. Alternatively, the effect of any input on a collection can be assessed and serve as a fingerprint of the effects of such input. Subarrays and collections produced under a variety of arrays or using cells from selected tissues or organs or other subset of the genome or from disease tissue and non-diseased cells, such as caner cells and non-cancerous cells from the same tissue, are also provided. The resulting collections of responding cells can provide fingerprints or signatures for known inputs (perturbations; conditions).

A variety of regulatory regions identified by the methods herein are also provided. Collections of cells that contain the regulatory regions operatively linked to nucleic acid encoding a reporter are also provided.

Collections of cells containing all of the identified promoters, each introduced into cells are provided. Also provided are collections in which the promoters are those that respond to a particular perturbation. The latter collections can be prepared from the former collections by sub-plating the first collection and identifying and selecting the cells that have promoters that respond to a particular condition.

Fully automated systems for screening cells, small molecules, antisense, RNA and other modulations, conditions and perturbations are provided. Computer systems and programs for directing the operation of the systems and/or for storing data from the screening assays are provided. Also provided are the resulting databases that contain information, such as the screened compounds, the regulatory regions and/or the cells.

### DESCRIPTION OF THE FIGURES

FIG. 1 depicts the cell-based assays provided herein showing the diversity of inputs that include small organics, combinatorial libraries, antibodies, natural products, genes, nucleic acid molecules and any other condition or perturbation that alters the state of a cell or alters gene expression, the hits that are produced by the assays and the variety of further analytical protocols that can be employed, and that the assays provide insights into biological processes and identification of targets of the input perturbations.

FIG. 2 sets forth retroviral transduction efficiencies for exemplary cell types and cellular processes that can be studied using each cell type.

### DETAILED DESCRIPTION

### A. Definitions

Unless defined otherwise, all technical and scientific terms used herein have the same meaning as is commonly understood by one of skill in the art to which the invention(s) belong. All patents, patent applications, published applications and publications, Genbank sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such indentifier or address, it understood that such identifiers can change and particular information on the internet can come and go, but equivalent information can be found by searching the internet. Reference thereto evidences the availability and public dissemination of such information.

As used herein, high-throughput screening (HTS) refers to processes that test a large number of samples, such as samples of test proteins or cells containing nucleic acids encoding the proteins of interest to identify structures of interest or the identify test compounds that interact with the variant proteins or cells containing them. HTS operations are amenable to automation and are typically computerized to handle sample preparation, assay procedures and the subsequent processing of large volumes of data.

As used herein, a perturbuation refers to any input that results in an altered cell response. Perturbations include any internal or external change in a cellular environment that results in an altered response compared to its absence. Thus, as used herein, a perturbation with reference to the cells refers to anything intra- or extra-cellular that alters gene expression or alters a cellular response. Perturbations include, but are not limited to, signals, such as those transduced by secondary messenger pathways, small effector molecules, including, for example, small organics, antisense, RNA and DNA, changes in intra or extracellular ion concentrations, such as changes in pH, Ca, Mg, Na and other ions, changes in temperature, pressure and concentration of any extracellular or intracellular component. Any such change or effector or condition is collectively referred to as a perturbation.

As used herein, signals refer to transduced signals, such as those initiated by binding or removal or other interaction of a ligand with a cell surface receptor. Extracellular signals include an molecule or a change in the environment that is transduced intracellularly via cell surface proteins that interact, directly or indirectly, with the signal. An extracellular signal or effector molecule is any compound or substance that in some manner specifically alters the activity of a cell surface protein. Examples of such signals include, but are not limited to, molecules such as acetylcholine, growth factors, hormones and other mitogenic substances, such as phorbol mistric acetate (PMA), that bind to cell surface receptors and ion channels and modulate the activity of such receptors and channels. For example, antagonists are extracellular signals that block or decrease the activity of cell surface protein and agonists are examples of extracellular signals that potentiate, induce or otherwise enhance the activity of cell surface proteins.

As used herein, extracellular signals also include as yet unidentified substances that modulate the activity of a cell surface protein and thereby affect intracellular functions and that are potential pharmacological agents that can be used to treat specific diseases by modulating the activity of specific cell surface receptors.

As used herein, “reporter” or “reporter moiety” refers to any moiety that allows for the detection of a molecule of interest, such as a protein expressed by a cell. Typical reporter moieties include, include, for example, fluorescent proteins, such as red, blue and green fluorescent proteins (see, e.g., U.S. Pat. No. 6,232,107, which provides GFPs from Renilla species and other species), the lacZ gene from E. coli, alkaline phosphatase, chloramphenicol acetyl transferase (CAT) and other such well-known genes. For expression in cells, nucleic acid encoding the reporter moiety can be expressed as a fusion protein with a protein of interest or under to the control of a promoter of interest. For the methods herein, reporters that are identifiable visually with a light detecting device are conveniently used. Patterns of light resulting from exposure of a collection of cells to a perturbation can be readily observed and saved as an image or a form derived therefrom. Pattern recognition software is optionally employed to identify resulting patterns.

As used herein, identifying the target “for an effector” means finding an appropriate protein traget to screen perturbation, such as a small molecule modulator of that protein. In essence, the method provides a means for rational target selection by altering concentrations of components of pathways and observing the phenotypic results to permit identification of the rate limiting step(s) in a pathway. Typically the rate limiting step(s) is targeted.

As used herein, identifying the target “of an effector” or “of a perturbation” means having a perturbations, such as an effector or condition, that has a known effect and then finding the target that mediates the effect.

As used herein, chemiluminescence refers to a chemical reaction in which energy is specifically channeled to a molecule causing it to become electronically excited and subsequently to release a photon thereby emitting visible light. Temperature does not contribute to this channeled energy. Thus, chemiluminescence involves the direct conversion of chemical energy to light energy. Bioluminescence refers to the subset of chemiluminescence reactions that involve luciferins and luciferases (or the photoproteins). Bioluminescence does not herein include phosphorescence.

As used herein, bioluminescence, which is a type of chemiluminescence, refers to the emission of light by biological molecules, particularly proteins. The essential condition for bioluminescence is molecular oxygen, either bound or free in the presence of an oxygenase, a luciferase, which acts on a substrate, a luciferin. Bioluminescence is generated by an enzyme or other protein (luciferase) that is an oxygenase that acts on a substrate luciferin (a bioluminescence substrate) in the presence of molecular oxygen and transforms the substrate to an excited state, which upon return to a lower energy level releases the energy in the form of light.

As used herein, the substrates and enzymes for producing bioluminescence are generically referred to as luciferin and luciferase, respectively. When reference is made to a particular species thereof, for clarity, each generic term is used with the name of the organism from which it derives, for example, bacterial luciferin or firefly luciferase.

As used herein, luciferase refers to oxygenases that catalyze a light emitting reaction. For instance, bacterial luciferases catalyze the oxidation of flavin mononucleotide (FMN) and aliphatic aldehydes, which reaction produces light. Another class of luciferases, found among marine arthropods, catalyzes the oxidation of Cypridina (Vargula) luciferin, and another class of luciferases catalyzes the oxidation of Coleoptera luciferin.

Thus, luciferase refers to an enzyme or photoprotein that catalyzes a bioluminescent reaction (a reaction that produces bioluminescence). The luciferases, such as firefly and Renilla luciferases, that are enzymes which act catalytically and are unchanged during the bioluminescence generating reaction. The luciferase photoproteins, such as the aequorin and obelin photoproteins to which luciferin is non-covalently bound, are changed, such as by release of the luciferin, during bioluminescence generating reaction. The luciferase is a protein that occurs naturally in an organism or a variant or mutant thereof, such as a variant produced by mutagenesis that has one or more properties, such as thermal or pH stability, that differ from the naturally-occurring protein. Luciferases and modified mutant or variant forms thereof are well known.

Thus, reference, for example, to “Renilla luciferase” means an enzyme isolated from member of the genus Renilla or an equivalent molecule obtained from any other source, such as from another Anthozoa, or that has been prepared synthetically. The luciferases and luciferin and activators thereof are referred to as bioluminescence generating reagents or components. As used herein, the component luciferases, luciferins, and other factors, such as O₂, Mg²⁺, Ca²⁺ are also referred to as bioluminescence generating reagents (or agents or components).

As used herein, a promoter region refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences can be cis acting or can be responsive to trans acting factors. Promoters, depending upon the nature of the regulation, can be constitutive or regulated.

As used herein, the term “regulatory region” means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present, or at increased concentration, gene expression increases. Regulatory regions also include sequences that confer repression of gene expression (i.e., a substance or stimulus decreases transcription). When a repressor is present or at increased concentration, gene expression decreases. Regulatory regions are known to influence, modulate or control many in vivo biological activities including cell proliferation, cell growth and death, cell differentiation and immune-modulation. Regulatory regions typically bind one or more trans-acting proteins which results in either increased or decreased transcription of the gene.

Particular examples of gene regulatory regions are promoters and enhancers. Promoters are sequences located around the transcription or translation start site, typically positioned 5′ f the translation start site. Promoters usually are located within 1 Kb of the translation start site, but can be located further away, for example, 2 Kb, 3 Kb, 4 Kb, 5 Kb or more, up to an including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding sites (IRES) elements for the creation of multigene, or polycistronic, messages, polyadenylation signals to provide proper polyadenylation of the transcript of a gene of interest and stop codons and can be optionally included in an expression vector.

As used herein, regulatory molecule refers to a polymer of deoxyribonucleic acid (DNA) or ribonucleic acid (RNA), or an oligonucleotide mimetic, or a polypeptide or other molecule that is capable of enhancing or inhibiting expression of a gene.

As used herein, the phrase “operatively linked” generally means the sequences or segments have been covalently joined into one piece of DNA, whether in single or double stranded form, whereby control or regulatory sequences on one segment control or permit expression or replication or other such control of other segments. The two segments are not necessarily contiguous. It means a juxtaposition between two or more components so that the components are in a relationship permitting them to function in their intended manner. Thus, in the case of a regulatory region operatively linked to a reporter or any other polynucleotide, or a reporter or any polynucleotide operatively linked to a regulatory region, expression of the polynucleotide/reporter is influenced or controlled (e.g., modulated or altered, such as increased or decreased) by the regulatory region. For gene expression a sequence of nucleotides and a regulatory sequence(s) are connected in such a way to control or permit gene expression when the appropriate molecular signal, such as transcriptional activator proteins, are bound to the regulatory sequence(s). Operative linkage of heterologous nucleic acid, such as DNA, to regulatory and effector sequences of nucleotides, such as promoters, enhancers, transcriptional and translational stop sites, and other signal sequences refers to the relationship between such DNA and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA in reading frame.

As used herein, a responder gene is a gene whose expression increases or decreases when a cell containing the gene or the gene is exposed to a perturbation, such as a small effector molecule, an extracellular signal, and a change in environment. Cells from an organism, or a tissue or an organ or other are exposed to a perturbation, and genes that have altered expression are identified. The genes that respond to the perturbation are referred to as responder genes. Exposure to different perturbations will yield different sets of genes that are responders. In some embodiments, responders to a plurality of perturbations are identified; in other embodiments, responders to a selected or particular perturbation, or from a particular cell type are selected. Subsets of the responder genes also can be identified. Once the responder genes are identified, regulatory regions, such as regions containing promoters, enhancers, transcription factor binding sites, translational regulatory regions, silencers and other such regulatory regions, are identified and isolated. The regulatory regions are each linked to nucleic acid encoding a reporter or to a nucleic acid reporter, and are introduced into cells. The resulting collection of cells is a collection of responder cells. Generally the collection is addressable (i.e., the identity of the regulatory region in each cell is known), such as by position on a substrate. Sub-collections of cells with different response patterns can be identified.

As used herein, robust responders refer to genes whose expression is increased or decreased substantially in response to a substance or stimulus. What is substantial depends upon the assay and reporting moiety. The precise increase, which can be empirically determined for each assay and/or collection of cells, should be sufficient to render the signals from reporters expressed from nucleic acid operatively linked to a robust responder regulatory region detectable under the conditions of the assay. Typically at least two-fold, generally at least a three-fold increase compared to other genes expressed under the same perturbations and/or compared to the regulatory region in the absence of the perturbations.

As used herein, receptor refers to a biologically active molecule that specifically binds to (or with) other molecules. The term “receptor protein” can be used to more specifically indicate the proteinaceous nature of a specific receptor. A receptor refers to a molecule that has an affinity for a given ligand. Receptors can be naturally-occurring or synthetic molecules. Receptors also can be referred to in the art as anti-ligands. As used herein, the receptor and anti-ligand are interchangeable. Receptors can be used in their unaltered state or as aggregates with other species. Receptors can be attached, covalently or noncovalently, or in physical contact with, to a binding member, either directly or indirectly via a specific binding substance or linker. Examples of receptors, include, but are not limited to: antibodies, cell membrane receptors, cell surface receptors and internalizing receptors, monoclonal antibodies and antisera reactive with specific antigenic determinants (such as on viruses, cells, or other materials), drugs, polynucleotides, nucleic acids, peptides, cofactors, lectins, sugars, polysaccharides, cells, cellular membranes, and organelles.

Examples of receptors and applications using such receptors, include but are not restricted to:

a) enzymes: specific transport proteins or enzymes essential to survival of microorganisms, which could serve as targets for antibiotic (ligand) selection;

b) antibodies: identification of a ligand-binding site on the antibody molecule that combines with the epitope of an antigen of interest can be investigated; determination of a sequence that mimics an antigenic epitope can lead to the development of vaccines of which the immunogen is based on one or more of such sequences or lead to the development of related diagnostic agents or compounds useful in therapeutic treatments such as for auto-immune diseases

c) nucleic acids: identification of ligand, such as protein or RNA, binding sites;

d) catalytic polypeptides: polymers, preferably polypeptides, that are capable of promoting a chemical reaction involving the conversion of one or more reactants to one or more products; such polypeptides generally include a binding site specific for at least one reactant or reaction intermediate and an active functionality proximate to the binding site, in which the functionality is capable of chemically modifying the bound reactant (see, e.g., U.S. Pat. No. 5,215,899);

e) hormone receptors: determination of the ligands that bind with high affinity to a receptor is useful in the development of hormone replacement therapies; for example, identification of ligands that bind to such receptors can lead to the development of drugs to control blood pressure; and

f) opiate receptors: determination of ligands that bind to the opiate receptors in the brain is useful in the development of less-addictive replacements for morphine and related drugs.

As used herein, antibody includes antibody fragments, such as Fab fragments, which are composed of a light chain and the variable region of a heavy chain.

As used herein, a ligand is a molecule that is specifically recognized by a particular receptor. Examples of ligands, include, but are not limited to, agonists and antagonists for cell membrane receptors, toxins and venoms, viral epitopes, hormones, such as steroids), hormone receptors, opiates, peptides, enzymes, enzyme substrates, cofactors, drugs, lectins, sugars, oligonucleotides, nucleic acids, oligosaccharides, proteins, and monoclonal antibodies.

As used herein, an anti-ligand is a molecule that has a known or unknown affinity for a given ligand and can be immobilized on a predefined region. Anti-ligands can be naturally-occurring or manmade molecules. Also, they can be employed in their unaltered state or as aggregates with other species. Anti-ligands can be reversibly attached, covalently or noncovalently, to a binding member, either directly or via a specific binding substance. By “reversibly attached” is meant that the binding of the anti-ligand (or specific binding member or ligand) is reversible and has, therefore, a substantially non-zero reverse, or unbinding, rate. Such reversible attachments can arise from noncovalent interactions, such as electrostatic forces, van der Waals forces, hydrophobic (i.e., entropic) forces and other forces. Furthermore, reversible attachments also can arise from certain, but not all covalent bonding reactions. Examples include, but are not limited to, attachment by the formation of hemiacetals, hemiketals, imines, acetals and ketals (see, e.g., Morrison et al. (1966) “Organic Chemistry”, 2nd ed., ch. 19). Examples of anti-ligands which can be employed in the methods and devices herein include, but are not limited to, cell membrane receptors, monoclonal antibodies and antisera reactive with specific antigenic determinants (such as on viruses, cells or other materials), hormones, drugs, oligonucleotides, peptides, peptide nucleic acids, enzymes, substrates, cofactors, lectins, sugars, oligosaccharides, cells, cellular membranes, and organelles.

As used herein, small amounts of nucleic acid (or protein) mean sub microgram amounts, including picogram and fentamole amounts.

As used herein, the term vector refers to a nucleic acid molecule capable of transporting another nucleic acid to which it has been linked, and include, but are not limited to, plasmids, cosmids and vectors of virus origin. Ioning vectors are typically used to genetically manipulate gene sequences while expression vectors are used to express the linked nucleic acid in a cell in vitro, ex vivo or in vivo. A vector that remains episomal contains at least an origin of replication for propagation in a cell; other vectors, such as retroviral vectors integrate into a host cell chromosome. One type of vector is an episome, i.e., a nucleic acid capable of extra-chromosomal replication.

Other vectors include are those capable of autonomous replication and/or expression of nucleic acids to which they are linked. Vectors capable of directing the expression of genes to which they are operatively linked are referred to herein as “expression vectors”. An “expression vector” therefore includes a gene regulatory region operatively linked to a sequence such as a reporter and can be propagated in cells. An “expression vector” can contain an origin of replication for propagation in a cell and includes a control element so that expression of a gene operatively linked thereto is influenced by the control element. Control elements include gene regulatory regions (e.g., promoters, transcription factor binding sites and enhancer elements) as set forth herein, that facilitate or direct or control transcription of an operatively linked sequence. “Plasmid” and “vector” are used interchangeably as the plasmid is the most commonly used form of vector. Other such other forms of expression vectors that serve equivalent functions and that become known in the art subsequently hereto. Vectors can include a selection marker.

As used herein, “selection marker” means a gene that allows selection of cells containing the gene. “Positive selection” means that only cells that contain the selection marker will survive upon exposure to the positive selection agent. For example, drug resistance is a common positive selection marker; cells containing a drug resistance gene will survive in culture medium containing the selection drug; whereas those which do not contain the resistance gene will die. Suitable drug resistance genes are neo, which confers resistance to G418, hygr, which confers resistance to hygromycin and puro, which confers resistance to puromycin. Other positive selection marker genes include reporter genes that allow identification by screening of cells. These genes include genes for fluorescent proteins (GFP), the lacZ gene (β-galactosidase), the alkaline phosphatase gene, and chlorampehnicol acetyl transferase. Vectors provided herein can contain negative selection markers.

As used herein, “negative selection” means that cells containing a negative selection marker are killed upon exposure to an appropriate negative selection agent. For example, cells which contain the herpes simplex virus-thymidine kinase (HSV-tk) gene are sensitive to the drug gancyclovir (GANC). Similarly, the gpt gene renders cells sensitive to 6-thioxanthine.

As used herein, self-inactivating (“SIN”) retroviral vectors are replication-deficient vectors that are created by deleting the promoter and enhancer sequences from the U3 region of the 3′ LTR (see, e.g., Yu et al. (1986) Proc. Natl. Acad. Sci. U.S.A. 83:3194-3198). Self-inactivating retrovirus have the 3′LTR and U3 regions removed so that upon recombination the LTR is gone. A functional U3 region in the 5′ LTR permits expression of a recombinant viral genome in appropriate packaging lines. Upon expression of its genomic RNA and reverse transcription into cDNA, the U3 region of the 5′ LTR of the original provirus is deleted and replaced with defective U3 region of the 3′ LTR. As a result, when a SIN vector integrates, the non-functional 3′ LTR replaces the functional 5′ LTR U3 region, rendering the virus incapable of expressing the full-length genomic transcript.

As used herein, “expression cassette” means a polynucleotide sequence containing a gene operatively linked to a control element (i.e. gene regulatory region) that can be transcribed and, if appropriate, translated. A gene regulatory region expression cassette includes a gene regulatory region of a responder, such as a robust responder, gene operatively linked to a sequence that encodes a reporter.

As used herein, a unidirection blocking sequence (utb) is a sequence of nucleotides that blocks expression of downstream nucleic acids (see, e.g., U.S. Pat. No. 5,583,022). A utb avoids antisense effects created by two promoters that are on opposite strands.

As used herein, a scaffold attachment region (SAR) or a sequence that reduces or prevents nearby chromatin or adjacent sequences from influencing a promoter's control of the reporter gene. SARs insulate chromatin from nearby silencers and enhancers. In the constructs and vectors herein, a SAR insulates the reporter construct from other genes. A SAR is not transcribed or translated, it is not a promoter or enhancer element. Its affect on gene expression is primarily position independent (see, U.S. Pat. No. 6,194,212, which describes the identification and use of SARs in retroviral vectors). Typically a SAR is at least 450 base pairs (bp) in length, generally from 600-1000 bp, such as about 800 bp. The SAR generally is AT-rich (i.e., more than 50%, typically more than 70% of the bases are adenine or thymine), and will generally include repeated 4-6 bp motifs, e.g., ATTA, ATTTA, ATTTTA, TAAT, TAAAT, TAAAAT, TAATA, andlor ATATTT, separated by spacer sequences, such as 3-20 bp, usually 8-12 bp, in length. The SAR can be from any eukaryote, such as a mammal, including a human. Suitably the SAR is the SAR for human IFN-β gene or a fragment thereof, such as a SAR derived from or corresponding to the 5′ SAR of human interferon beta (IFN-β) (see, Klehr et al. (1991) Biochemistry 30:1264-1270), including a fragment of at least 50 base pairs (bp) in length, typically from 600-1000 bp, such as about 800 bp, and being substantially identical to a corresponding portion of the 5′ SAR of a human IFN-βgene. By corresponding is meant having at least 80% (i.e., 8 out of every 10 base pairs is the same), generally at least 90% or 95% identity therewith. An exemplary SAR is the 800 bp Eco-RI-HindIII (blunt end) fragment of the 5′ SAR element of IFN-β (see, Mielke et al.(1990) Biochemistry 29:7475-7485) or one that is at least 80%, 90%, and 95% identical thereto.

As used herein, position independent means that functioning of a sequence does not require insertion into a specific site, but such sequence cannot be inserted such that other functioning sequences are destroyed.

Solid Supports, Chips, Arrays and Collection

As used herein, a collection contains two, generally three, or more elements.

As used herein, an array refers to a collection of elements, such as cells and nucleic acid molecules, containing three or more members; arrays can be in solid phase or liquid phase. An addressable array or collection is one in which each member of the collection is identifiable typically by position on a solid phase support or by virtue of an identifiable or detectable label, such as by color, fluorescence, electronic signal (i.e. RF, microwave or other frequency that does not substantially alter the interaction of the molecules of interest), bar code or other symbology, chemical or other such label. Hence, in general the members of the array are immobilized to discrete identifiable loci on the surface of a solid phase or directly or indirectly linked to or otherwise associated with the identifiable label, such as affixed to a microsphere or other particulate support (herein referred to as beads) and suspended in solution or spread out on a surface. The collection can be in the liquid phase if other discrete identifiers, such as chemical, electronic, colored, fluorescent or other tags are included.

As used herein, a substrate (also referred to as a matrix support, a matrix, an insoluble support, a support or a solid support) refers to any solid or semisolid or insoluble support to which a molecule of interest, typically a biological molecule, organic molecule or biospecific ligand is linked or contacted. A substrate or support refers to any insoluble material or matrix that is used either directly or following suitable derivatization, as a solid support for chemical synthesis, assays and other such processes. Substrates contemplated herein include, for example, silicon substrates or siliconized substrates that are optionally derivatized on the surface intended for linkage of anti-ligands and ligands and other macromolecules. Other substrates are those on which cells adhere.

Such materials include any materials that are used as affinity matrices or supports for chemical and biological molecule syntheses and analyses, such as, but are not limited to: polystyrene, polycarbonate, polypropylene, nylon, glass, dextran, chitin, sand, pumice, agarose, polysaccharides, dendrimers, buckyballs, polyacrylamide, silicon, rubber, and other materials used as supports for solid phase syntheses, affinity separations and purifications, hybridization reactions, immunoassays and other such applications.

Thus, a substrate, support or matrix refers to any solid or semisolid or insoluble support on which the molecule of interest, typically a biological molecule, macromolecule, organic molecule or biospecific ligand or cell is linked or contacted. Typically a matrix is a substrate material having a rigid or semi-rigid surface. In many embodiments, at least one surface of the substrate is substantially flat or is a well, although in some embodiments it can be desirable to physically separate synthesis regions for different polymers with, for example, wells, raised regions, etched trenches, or other such topology. Matrix materials include any materials that are used as affinity matrices or supports for chemical and biological molecule syntheses and analyses, such as, but are not limited to: polystyrene, polycarbonate, polypropylene, nylon, glass, dextran, chitin, sand, pumice, polytetrafluoroethylene, agarose, polysaccharides, dendrimers, buckyballs, polyacrylamide, Kieselguhr-polyacrlamide noncovalent composite, polystyrene-polyacrylamide covalent composite, polystyrene-PEG (polyethyleneglycol) composite, silicon, rubber, and other materials used as supports for solid phase syntheses, affinity separations and purifications, hybridization reactions, immunoassays and other such applications.

The substrate, support or matrix herein can be particulate or can be a be in the form of a continuous surface, such as a microtiter dish or well, a glass slide, a silicon chip, a nitrocellulose sheet, nylon mesh, or other such materials. When particulate, typically the particles have at least one dimension in the 5-10 mm range or smaller. Such particles, referred collectively herein as “beads”, are often, but not necessarily, spherical. Such reference, however, does not constrain the geometry of the matrix, which can be any shape, including random shapes, needles, fibers, and elongated. Roughly spherical “beads”, particularly microspheres that can be used in the liquid phase, are also contemplated. The “beads” can include additional components, such as magnetic or paramagnetic particles (see, e.g., Dyna beads (Dynal, Oslo, Norway)) for separation using magnets, as long as the additional components do not interfere with the methods and analyses herein. For the collections of cells, the substrate should be selected so that it is addressable (i.e., identifiable) and such that the cells are linked, absorbed, adsorboed or otherwise retained thereon.

As used herein, matrix or support particles refers to matrix materials that are in the form of discrete particles. The particles have any shape and dimensions, but typically have at least one dimension that is 100 mm or less, 50 mm or less, 10 mm or less, 1 mm or less, 100 μm or less, 50 μm or less and typically have a size that is 100 mm³ or less, 50 mm³ or less, 10 mm³ or less, and 1 mm³ or less, 100 μm³ or less and can be order of cubic microns. Such particles are collectively called “beads.”

As used herein, high density arrays refer to arrays that contain 384 or more, including 1536 or more or any multiple of 96 or other selected base, loci per support, which is typically about the size of a standard 96 well microtiter plate. Each such array is typically, although not necessarily, standardized to be the size of a 96 well microtiter plate. It is understood that other numbers of loci, such as 10, 100, 200, 300, 400, 500, 10ⁿ, wherein n is any number from 0 and up to 10 or more. Ninety-six is merely an exemplary number. For addressable collections that are homogeneous (i.e. not affixed to a solid support), the numbers of members are generally greater. Such collections can be labeled chemically, electronically (such as with radio-frequency, microwave or other detectable electromagnetic frequency that does not substantially interfere with a selected assay or biological interaction).

As used herein, the attachment layer refers the surface of the chip device to which molecules are linked. A chip can be a silicon semiconductor device, which is coated on a least a portion of the surface to render it suitable for linking molecules and inert to any reactions to which the device is exposed. Molecules are linked either directly or indirectly to the surface, linkage can be effected by absorption or adsorption, through covalent bonds, ionic interactions or any other interaction. Where necessary the attachment layer is adapted, such as by derivatization for linking the molecules.

As used herein, a gene chip, also called a genome chip and a microarray, refers to high density oligonucleotide-based arrays. Such chips typically refer to arrays of oligonucleotides for designed monitoring an entire genome, but can be designed to monitor a subset thereof. Gene chips contain arrayed polynucleotide chains (oligonucleotides of DNA or RNA or nucleic acid analogs or combinations thereof) that are single-stranded, or at least partially or completely single-stranded prior to hybridization. The oligonucleotides are designed to specifically and generally uniquely hybridize to particular polynucleotides in a population, whereby by virtue of formation of a hybrid the presence of a polynucleotide in a population can be identified. Gene chips are commercially available or can be prepared. Exemplary microarrays include the Affymetrix GeneChip® arrays. Such arrays are typically fabricated by high speed robotics on glass, nylon or other suitable substrate, and include a plurality of probes (oligonucleotides) of known identity defined by their address in (or on) the array (an addressable locus). The oligonucleotides are used to determine complementary binding and to thereby provide parallel gene expression and gene discovery in a sample containing target nucleic acid molecules. Thus, as used herein, a gene chip refers to an addressable array, typically a two-dimensional array, that includes plurality of oligonucleotides associate with addressable loci “addresses”, such as on a surface of a microtiter plate or other solid support.

As used herein, a plurality of genes includes at least two, five, 10, 25, 50, 100, 250, 500, 1000, 2,500, 5,000, 10,000, 100,000, 1,000,000 or more genes. A plurality of genes can include complete or partial genomes of an organism or even a plurality thereof. Selecting the organism type determines the genome from among which the gene regulatory regions are selected. Exemplary organisms for gene screening include animals, such as mammals, including human and rodent, such as mouse, insects, yeast, bacteria, parasites, and plants.

As used herein, a transcriptome is a collection of transcripts from a genome, such a collection from a particular organ, cell, tissue, cell(s) or pathway. A transcriptome is a collection of RNA molecules (or cDNA produced therefrom) present in a cell, tissue or organ or other selected component of an animal or plant or other organism (see, e.g., Hoheisel et al. (1997) Trends Biotechnol. 15:465-469; Velculescu (1997) Cell 88:243-251 (1997).

Recombinases

As used herein, recognition sequences are particular sequences of nucleotides that a protein, DNA, or RNA molecule, such as, but are not limited to, a restriction endonuclease, a modification methylase and a recombinase) recognizes and binds. For example, a recognition sequence for Cre recombinase (see, e.g., SEQ ID 46 is a 34 base pair sequence containing two 13 base pair inverted repeats (serving as the recombinase binding sites) flanking an 8 base pair core and designated loxP (see, e.g., Sauer (1994) Current Opinion in Biotechnology 5:521-527).

As used herein, a recombinase is an enzyme that catalyzes the exchange of DNA segments at specific recombination sites. An integrase herein refers to a recombinase that is a member of the lambda (λ) integrase family.

As used herein, recombination proteins include excisive proteins, integrative proteins, enzymes, co-factors and associated proteins that are involved in recombination reactions using one or more recombination sites (see, Landy (1993) Current Opinion in Biotechnology 3:699-707).

As used herein the expression “lox site” means a sequence of nucleotides at which the gene product of the cre gene, referred to herein as Cre, can catalyze a site-specific recombination. A LoxP site is a 34 base pair nucleotide sequence from bacteriophage P1 (see, e.g., Hoess et al. (1982) Proc. Natl. Acad. Sci. U.S.A. 79:3398-3402). The LoxP site contains two 13 base pair inverted repeats separated by an 8 base pair spacer region as follows: (SEQ ID NO. 46):

ATAACTTCGTATA ATGTATGC TATACGAAGTTAT

E. coliDH5Δlac and yeast strain BSY23 transformed with plasmid pBS44 carrying two loxP sites connected with a LEU2 gene are available from the American Type Culture Collection (ATCC) under accession numbers ATCC 53254 and ATCC 20773, respectively. The lox sites can be isolated from plasmid pBS44 with restriction enzymes Eco RI and Sal I, or Xho I and Bam I. In addition, a preselected DNA segment can be inserted into pBS44 at either the Sal I or Bam I restriction enzyme sites. Other lox sites include, but are not limited to, LoxB, LoxL, LoxC2 and LoxR sites, which are nucleotide sequences isolated from E. coli (see, e.g., Hoess et al. (1982) Proc. Natl. Acad. Sci. U.S.A. 79:3398). Lox sites also can be produced by a variety of synthetic techniques (see, e.g., Ito et al. (1982) Nuc. Acid Res. 10:1755 and Ogilvie et al. (1981) Science 270:270.

As used herein, the expression “cre gene” means a sequence of nucleotides that encodes a gene product that effects site-specific recombination of DNA in eukaryotic cells at lox sites. One cre gene can be isolated from bacteriophage P1 (see, e.g., Abremski et al. (1983) Cell 32:1301-1311). E. coli DH1 and yeast strain BSY90 transformed with plasmid pBS39 carrying a cre gene isolated from bacteriophage P1 and a GAL1 regulatory nucleotide sequence are available from the American Type Culture Collection (ATCC) under accession numbers ATCC 53255 and ATCC 20772, respectively. The cre gene can be isolated from plasmid pBS39 with restriction enzymes Xho I and Sal I.

As used herein, site specific recombination refers site specific recombination that is effected between two specific sites on a single nucleic acid molecule or between two different molecules that requires the presence of an exogenous protein, such as an integrase or recombinase.

For example, Cre-lox site-specific recombination includes the following three events:

a. deletion of a pre-selected DNA segment flanked by lox sites;

b. inversion of the nucleotide sequence of a pre-selected DNA segment flanked by lox sites; and

c. reciprocal exchange of DNA segments proximate to lox sites located on different DNA molecules.

This reciprocal exchange of DNA segments can result in an integration event if one or both of the DNA molecules are circular. DNA segment refers to a linear fragment of single- or double-stranded deoxyribonucleic acid (DNA), which can be derived from any source. Since the lox site is an asymmetrical nucleotide sequence, two lox sites on the same DNA molecule can have the same or opposite orientations with respect to each other. Recombination between lox sites in the same orientation result in a deletion of the DNA segment located between the two lox sites and a connection between the resulting ends of the original DNA molecule. The deleted DNA segment forms a circular molecule of DNA. The original DNA molecule and the resulting circular molecule each contain a single lox site. Recombination between lox sites in opposite orientations on the same DNA molecule result in an inversion of the nucleotide sequence of the DNA segment located between the two lox sites. In addition, reciprocal exchange of DNA segments proximate to lox sites located on two different DNA molecules can occur. All of these recombination events are catalyzed by the gene product of the cre gene. Thus, the Cre-lox system has can be used to specifically excise, delete or insert DNA. The precise event is controlled by the orientation of lox DNA sequences, in cis the lox sequences direct the Cre recombinase to either delete (lox sequences in direct orientation) or invert (lox sequences in inverted orientation) DNA flanked by the sequences, while in trans the lox sequences can direct a homologous recombination event resulting in the insertion of a recombinant DNA.

General Definitions

As used herein, biological and pharmacological activity includes any activity of a biological pharmaceutical agent and includes, but is not limited to, biological efficiency, transduction efficiency, gene/transgene expression, differential gene expression and induction activity, titer, progeny productivity, toxicity, cytotoxicity, immunogenicity, cell proliferation and/or differentiation activity, anti-viral activity, morphogenetic activity, teratogenetic activity, pathogenetic activity, therapeutic activity, tumor suppressor activity, ontogenetic activity, oncogenetic activity, enzymatic activity, pharmacological activity, cell/tissue tropism and delivery.

As used herein, “loss-of-function” sequence, as it refers to the effect of a polynucleotide such as antisense nucleic acid, siRNA and cDNA, refers to those sequences which, when expressed in a host cell, inhibit expression of a gene or otherwise render the gene product thereof to have substantially reduced activity, or preferably no activity relative to one or more functions of the corresponding wild-type gene product.

As used herein, phenotype refers to the physical or other manifestation of a genotype (a sequence of a gene). In the methods herein, phenotypes that result from alteration of a genotype are assessed.

As used herein, the amino acids, which occur in the various amino acid sequences appearing herein, are identified according to their known, three-letter or one-letter abbreviations (see, Table 1). The nucleotides, which occur in the various nucleic acid fragments, are designated with the standard single-letter designations used routinely in the art.

As used herein, amino acid residue refers to an amino acid formed upon chemical digestion (hydrolysis) of a polypeptide at its peptide linkages. The amino acid residues described herein are presumed to be in the “L” isomeric form. Residues in the “D” isomeric form, which are so-designated, can be substituted for any L-amino acid residue, as long as the desired functional property is retained by the polypeptide; such residues. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. § § 1.821-1.822, abbreviations for amino acid residues are shown in the following Table:

TABLE 1
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence and modified and unusual amino acids, such as those referred to in 37 C.F.R. § § 1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. (1987) Molecular Biology of the Gene, 4th Edition, The Benjamin/Cummings Pub. co., p.224).

Such substitutions are preferably made in accordance with those set forth in TABLE 2 as follows:

TABLE 2

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions are also permissible and can be determined empirically or in accord with known conservative substitutions.

As used herein, a biopolymer includes, but is not limited to, nucleic acid, proteins, polysaccharides, lipids and other macromolecules. Nucleic acids include DNA, RNA, and fragments thereof. Nucleic acids can be isolated or derived from genomic DNA, RNA, mitochondrial nucleic acid, chloroplast nucleic acid and other organelles with separate genetic material or can be prepared synthetically.

As used herein, nucleic acids include DNA, RNA and analogs thereof, including protein nucleic acids (PNA) and mixture thereof. Nucleic acids can be single or double stranded. When referring to probes or primers, optionally labeled with a detectable label, such as a fluorescent or radiolabel, single-stranded molecules are contemplated. Such molecules are typically of a length such that they are statistically unique or low copy number (typically less than 5 or 6, generally less than 3 copies in a library) for probing or priming a library. Generally a probe or primer contains at least 14, 16 or 30 contiguous nucleotides from a selected sequence thereof complementary to or identical to a polynucleotide of interest. Probes and primers can be 10, 14, 16, 20, 30, 50, 100 or more nucleic acid bases long.

As used herein, “oligonucleotide,” “polynucleotide” and “nucleic acid” include linear oligomers of natural or modified monomers or linkages, including deoxyribonucleosides, ribonucleotides, α-anomeric forms thereof capable of specifically binding to a target gene by way of a regular pattern of monomer-to-monomer interactions, such as Watson-Crick type of base pairing, base stacking, Hoogsteen or reverse Hoogsteen types of base pairing. Monomers are typically linked by phosphodiester bonds or analogs thereof to form the oligonucleotides. Whenever an oligonucleotide is represented by a sequence of letters, such as “ATGCCTG,” it is understood that the nucleotides are in a 5′→3′ order from left to right.

Typically oligonucleotides for hybridization include the four natural nucleotides; however, they also can include non-natural nucleotide analogs, derivatized forms or mimetics. Analogs of phosphodiester linkages include phosphorothioate, phosphorodithioate, phosphorandilidate, phosphoramidate, for example. A particular example of a mimetic is protein nucleic acid (see, e.g., Egholm et al. (1993) Nature 365:566; see also U.S. Pat. No. 5,539,083).

As used herein, labels include any composition or moiety that can be attached to or incorporated into nucleic acid that is detectable by spectroscopic, photochemical, biochemical, immunochemical, electrical, optical or chemical means. Exemplary labels include, but are not limited to, biotin for staining with labeled streptavidin conjugate, magnetic beads (e.g., DynabeadsTM), fluorescent dyes (e.g., 6-FAM, HEX, TET, TAMRA, ROX, JOE, 5-FAM, R110, fluorescein, texas red, rhodamine, phycoerythrin , lissamine, phycoerythrin (Perkin Elmer Cetus), Cy2, Cy3, Cy3.5, Cy5, Cy5.5, Cy7, FluorX (Amersham), radiolabels, enzymes (e.g., horse radish peroxidase, alkaline phosphatase and others used in ELISA), and colorimetric labels such as colloidal gold or colored glass or plastic (e.g., polystyrene, polypropylene, latex and other supports) beads, a fluorophore, a radioisotope or a chemiluminescent moiety.

As used herein, “mistmatch control” means a sequence that is not perfectly complementary to a particular oligonucleotide. The mismatch can include one or more mismatched bases. The mismatch(s) can be located at or near the center of the probe such that the mismatch is most likely to destabilize the duplex with the target sequence under hybridization conditions, but can be located anywhere, for example, a terminal mismatch. The mismatch control typically has a corresponding test probe that is perfectly complementary to the same particular target sequence. Mismatches are selected such that under appropriate hybridization conditions the test or control oligonucleotide hybridizes with its target sequence, but the mismatch oligonucleotide does not. Mismatch oligonucleotides therefore indicate whether hybridization is specific or not. For example, if the target gene is present the perfect match oligonucleotide should be consistently brighter than the mismatch oligonucleotide.

As used herein, nucleic acid derived from an RNA means that the RNA has ultimately served as a template. Thus, a cDNA reverse transcribed from an mRNA, an RNA transcribed from that cDNA, a DNA amplified from the cDNA, an RNA transcribed from the amplified DNA are derived from an RNA and using such derived products to determine changes in gene expression are included. Thus, suitable nucleic acids include, but are not limited to, mRNA transcripts of the gene or genes, cDNA reverse transcribed from the mRNA, cRNA transcribed from the cDNA, DNA amplified from the genes and RNA transcribed from amplified DNA.

As used herein, amplifying refers to means for increasing the amount of a biopolymer, especially nucleic acids. Based on the 5′ and 3′ primers that are chosen, amplification also serves to restrict and define the region of the genome, transcriptome or other same that is subject to analysis. Amplification can be by any means known to those skilled in the art, including use of the polymerase chain reaction (PCR) and other amplification protocols, such as ligase chain reaction, RNA replication, such as the autocatalytic replication catalyzed by, for example, Qβ replicase. Amplification is done quantitatively when the frequency of a polymorphism is determined.

As used herein, cleaving refers to non-specific and specific fragmentation of a biopolymer.

As used herein, by homologous means about greater than 25% nucleic acid or amino acid sequence identity, generally 25% 40%, 60%, 80%, 90% or 95%. The intended percentage will be specified. The terms “homology” and “identity” are often used interchangeably. In general, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073).

By sequence identity, the number of conserved amino acids are determined by standard alignment algorithms programs, and are used with default gap penalties established by each supplier. Substantially homologous nucleic acid molecules would hybridize typically at moderate stringency or at high stringency all along the length of the nucleic acid of interest. Also contemplated are nucleic acid molecules that contain degenerate codons in place of codons in the hybridizing nucleic acid molecule.

As used herein, a nucleic acid homolog refers to a nucleic acid that includes a preselected conserved nucleotide sequence, such as a sequence encoding a therapeutic polypeptide. By the term “substantially homologous” is meant having at least 80%, preferably at least 90%, most preferably at least 95% homology therewith or a less percentage of homology or identity and conserved biological activity or function. Ppolypeptide homologs would be polypeptides that could be encoded substantially identical (i.e., 80%, 90%, 95% identifical) sequences of nucleotides.

The terms “homology” and “identity” are often used interchangeably. In this regard, percent homology or identity can be determined, for example, by comparing sequence information using a GAP computer program. The GAP program uses the alignment method of Needleman and Wunsch (J. Mol. Biol. 48:443 (1970), as revised by Smith and Waterman (Adv. Appl. Math. 2:482 (1981). Briefly, the GAP program defines similarity as the number of aligned symbols (i.e., nucleotides or amino acids) which are similar, divided by the total number of symbols in the shorter of the two sequences. The preferred default parameters for the GAP program can include: (1) a unitary comparison matrix (containing a value of 1 for identities and 0 for non-identities) and the weighted comparison matrix of Gribskov and Burgess, Nucl. Acids Res. 14:6745 (1986), as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Whether any two nucleic acid molecules have nucleotide sequences that are, for example, at least 80%, 85%, 90%, 95%, 96%, 97%, 98% or 99%, “identical” can be determined using known computer algorithms such as the “FAST A” program, using for example, the default parameters as in Pearson and Lipman, Proc. Natl. Acad. Sci. USA 85:2444 (1988). Alternatively the BLAST function of the National Center for Biotechnology Information database can be used to determine identity. In general, sequences are aligned so that the highest order match is obtained. “Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. & Lipton, D., SIAM J Applied Math 48:1073 (1988)). Methods commonly employed to determine identity or similarity between two sequences include, but are not limited to, those disclosed in Guide to Huge Computers, Martin J. Bishop, ed., Academic Press, San Diego, 1994, and Carillo, H. & Lipton, D., SIAM J Applied Math 48:1073 (1988). Methods to determine identity and similarity are codified in computer programs. Preferred computer program methods to determine identity and similarity between two sequences include, but are not limited to, GCG program package (Devereux et al. (1984) Nucleic Acids Research 12(I):387), BLASTP, BLASTN, FASTA (Atschul, S. F., et al., J Molec Biol 215:403 (1990)), and CLUSTALW. For sequences displaying a relatively high degree of homology, alignment can be effected manually by simpling lining up the sequences by eye and matching the conserved portions.

Therefore, as used herein, the term “identity” represents a comparison between a test and a reference polypeptide or polynucleotide. For example, a test polypeptide can be defined as any polypeptide that is 90% or more identical to a reference polypeptide. Alignment can be performed with any program for such purpose using default gap parameters and penalties or those selected by the user. For example, a program called CLUSTALW program can be employed with parameters set as follows: scoring matrix BLOSUM, gap open 10, gap extend 0.1, gap distance 40% and transitions/transversions 0.5; specific residue penalties for hydrophobic amino acids (DEGKNPQRS), distance between gaps for which the penalties are augmented was 8, and gaps of extremities penalized less than internal gaps.

As used herein, substantially identical to a product means sufficiently similar so that the property of interest is sufficiently unchanged so that the substantially identical product can be used in place of the product.

As used herein, a “corresponding” position on a protein (or nucleic acid molecule) refers to an amino acid position (or nucleotide base position) based upon alignment to maximize sequence identity between or among related proteins(or nucleic acid molecules).

As used herein, the term at least “90% identical to” refers to percent identities from 90 to 100% relative to reference polypeptides or nucleic acid moleucles. Identity at a level of 90% or more is indicative of the fact that, assuming for exemplification purposes a test and reference polypeptide (or polynucleotide) length of 100 amino acids are compared. No more than 10% (i.e., 10 out of 100) amino acids in the test polypeptide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g. 10/100 amino acid difference (approximately 90% identity). Differences are defined as nucleic acid or amino acid substitutions, or deletions.

As used herein, it is also understood that the terms substantially identical or similar varies with the context as understood by those skilled in the relevant art.

As used herein, “hybridization” refers to the binding between complementary nucleic acids. “Selective hybridization” refers to hybridization that distinguishes related sequences from unrelated sequences. Hybridization conditions will be such that an oligonucleotide will hybridize to its target nucleic acid, but not significantly to non-target sequences. As is understood by those skilled in the art, the T_{M} (melting temperature) refers to the temperature at which binding between complementary sequences is no longer stable. For two nucleic acid sequences to bind, the temperature of a hybridization reaction must be less than the calculated T_{M} for the sequences. The T_{M} is influenced by the amount of sequence complementarity, length, composition (% GC), type of nucleic acid (RNA vs. DNA), and the amount of salt, detergent and other components in the reaction (e.g., formamide). For example, longer hybridizing sequences are stable at higher temperatures. Duplex stability between RNA, DNA and mixtures thereof is generally in the order of RNA:RNA>RNA:DNA>DNA:DNA. All of these factors are considered in establishing appropriate hybridization conditions (see, e.g., the hybridization techniques and formula for calculating T_{M} described in Sambrook et al. (1989) Molecular Cloning: A Laboratory Manual (2nd Ed.), Vol. 1-3, Cold Spring Harbor Laboratory, Cold Spring Harbor, N.Y.). Generally, stringent conditions are selected to be about 5° C. lower than the melting point (Tm) for the specific sequence at a defined ionic strength and pH.

Typically, wash conditions are adjusted so as to attain the desired degree of hybridization stringency. Thus, hybridization stringency can be determined empirically, for example, by washing under particular conditions, e.g., at low stringency conditions or high stringency conditions. Optimal conditions for selective hybridization will vary depending on the particular hybridization reaction involved. An exemplary gene chip hybridization is described in Example 1.

As used herein, to hybridize under conditions of a specified stringency is used to describe the stability of hybrids formed between two single-stranded DNA fragments and refers to the conditions of ionic strength and temperature at which such hybrids are washed, following annealing under conditions of stringency less than or equal to that of the washing step. Typically high, medium and low stringency encompass the following conditions or equivalent conditions thereto:

1) high stringency: 0.1×SSPE or SSC, 0.1% SDS, 65° C.

2) medium stringency: 0.2×SSPE or SSC, 0.1% SDS, 50° C.

3) low stringency: 1.0×SSPE or SSC, 0.1% SDS, 50° C. Equivalent conditions refer to conditions that select for substantially the same percentage of mismatch in the resulting hybrids. Additions of ingredients, such as formamide, Ficoll, and Denhardt's solution affect parameters such as the temperature under which the hybridization should be conducted and the rate of the reaction. Thus, hybridization in 5×SSC, in 20% formamide at 42° C. is substantially the same as the conditions recited above hybridization under conditions of low stringency. The recipes for SSPE, SSC and Denhardt's and the preparation of deionized formamide are described, for example, in Sambrook et al. (1989) Molecular Cloning, A Laboratory Manual, Cold Spring Harbor Laboratory Press, Chapter 8; see, Sambrook et al., vol. 3, p. B.13, see, also, numerous catalogs that describe commonly used laboratory solutions). It is understood that equivalent stringencies can be achieved using alternative buffers, salts and temperatures.

As used herein equivalent, when referring to two sequences of nucleic acids means that the two sequences in question encode the same sequence of amino acids or equivalent proteins. When “equivalent” is used in referring to two proteins or peptides, it means that the two proteins or peptides have substantially the same amino acid sequence with only conservative amino acid substitutions (see, e.g., Table 2) that do not substantially alter the activity or function of the protein or peptide. When “equivalent” refers to a property, the property does not need to be present to the same extent (e.g., peptides can exhibit different rates of the same type of enzymatic activity), but the activities are preferably substantially the same. “Complementary,” when referring to two nucleotide sequences, means that the two sequences of nucleotides are capable of hybridizing, preferably with less than 25%, more preferably with less than 15%, even more preferably with less than 5%, most preferably with no mismatches between opposed nucleotides. Preferably the two molecules will hybridize under conditions of high stringency.

As used herein, heterologous or foreign nucleic acid, such as DNA and RNA, are used interchangeably and refer to DNA or RNA that does not occur naturally as part of the genome in which it is present or which is found in a location or locations in the genome that differ from that in which it occurs in nature. Heterologous nucleic acid is generally not endogenous to the cell into which it is introduced, but has been obtained from another cell or prepared synthetically. Generally, although not necessarily, such nucleic acid encodes RNA and proteins that are not normally produced by a cell in which it is expressed. Any DNA or RNA that one of skill in the art would recognize or consider as heterologous or foreign to the cell in which it is expressed is herein encompassed by heterologous DNA. Heterologous DNA and RNA also can encode RNA or proteins that mediate or alter expression of endogenous DNA by affecting transcription, translation, or other regulatable biochemical processes. Examples of heterologous nucleic acid include, but are not limited to, nucleic acid that encodes traceable marker proteins, such as a protein that confers drug resistance, nucleic acid that encodes therapeutically effective substances, such as anti-cancer agents, enzymes and hormones, and DNA that encodes other types of proteins, such as antibodies.

Hence, herein heterologous DNA or foreign DNA, includes a DNA molecule not present in the exact orientation and position as the counterpart DNA molecule found in the genome. It also can refer to a DNA molecule from another organism or species (i.e., exogenous).

As used herein, a sequence complementary to at least a portion of an RNA, with reference to antisense oligonucleotides, means a sequence having sufficient complementarily to be able to hybridize with the RNA, preferably under moderate or high stringency conditions, forming a stable duplex. The ability to hybridize depends on the degree of complementarily and the length of the antisense nucleic acid. The longer the hybridizing nucleic acid, the more base mismatches it can contain and still form a stable duplex (or triplex, as the case can be). One skilled in the art can ascertain a tolerable degree of mismatch by use of standard procedures to determine the melting point of the hybridized complex.

As used herein, “isolated” with reference to a nucleic acid molecule or polypeptide or other biomolecule means that the nucleic acid or polypeptide has separated from the genetic environment from which the polypeptide or nucleic acid were obtained. It also can mean altered from the natural state. For example, a polynucleotide or a polypeptide naturally present in a living animal is not “isolated,” but the same polynucleotide or polypeptide separated from the coexisting materials of its natural state is “isolated”, as the term is employed herein. Thus, a polypeptide or polynucleotide produced and/or contained within a recombinant host cell is considered isolated. Also intended as an “isolated polypeptide” or an “isolated polynucleotide” are polypeptides or polynucleotides that have been purified, partially or substantially, from a recombinant host cell or from a native source. For example, a recombinantly produced version of a compounds can be substantially purified by the one-step method described in Smith and Johnson, Gene 67:31-40 (1988). The terms isolated and purified are sometimes used interchangeably.

Thus, by “isolated” is meant that the nucleic acid is free of the coding sequences of those genes that, in the naturally-occurring genome of the organism (if any) immediately flank the gene encoding the nucleic acid of interest. Isolated DNA can be single-stranded or double-stranded, and can be genomic DNA, cDNA, recombinant hybrid DNA, or synthetic DNA. It can be identical to a native DNA sequence, or can differ from such sequence by the deletion, addition, or substitution of one or more nucleotides.

“Isolated” or “purified” as it refers to preparations made from biological cells or hosts means any cell extract containing the indicated DNA or protein including a crude extract of the DNA or protein of interest. For example, in the case of a protein, a purified preparation can be obtained following an individual technique or a series of preparative or biochemical techniques and the DNA or protein of interest can be present at various degrees of purity in these preparations. The procedures can include for example, but are not limited to, ammonium sulfate fractionation, gel filtration, ion exchange change chromatography, affinity chromatography, density gradient centrifugation and electrophoresis.

A preparation of DNA or protein that is “substantially pure” or “isolated” should be understood to mean a preparation free from naturally occurring materials with which such DNA or protein is normally associated in nature. “Essentially pure” should be understood to mean a “highly” purified preparation that contains at least 95% of the DNA or protein of interest.

A cell extract that contains the DNA or protein of interest should be understood to mean a homogenate preparation or cell-free preparation obtained from cells that express the protein or contain the DNA of interest. The term “cell extract” is intended to include culture media, especially spent culture media from which the cells have been removed.

As used herein, “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, referred to as a single nucleotide polymorphism (SNP), the identity of which differs in different alleles. A polymorphic region also can be several nucleotides in length.

As used herein, “polymorphic gene” refers to a gene having at least one polymorphic region.

As used herein, “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is the to be heterozygous for the gene. Alleles of a specific gene can differ from each other in a single nucleotide, or several nucleotides, and can include substitutions, deletions, and insertions of nucleotides. An allele of a gene also can be a form of a gene containing a mutation.

As used herein, the term “gene” or “recombinant gene” refers to a nucleic acid molecule containing an open reading frame and including at least one exon and (optionally) an intron sequence. A gene can be either RNA or DNA. Genes can include regions preceding and following the coding region (leader and trailer).

As used herein, “intron” refers to a DNA sequence present in a given gene which is spliced out during mRNA maturation.

As used herein, “nucleotide sequence complementary to the nucleotide sequence set forth in SEQ ID No. x” refers to the nucleotide sequence of the complementary strand of a nucleic acid strand having SEQ ID No. x. The term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ ID No. x refers to the complementary strand of the strand having SEQ ID No. x or to any nucleic acid having the nucleotide sequence of the complementary strand of SEQ ID No. x. When referring to a single stranded nucleic acid having the nucleotide sequence SEQ ID No. x, the complement of this nucleic acid is a nucleic acid having a nucleotide sequence which is complementary to that of SEQ ID No. x.

As used herein, the term “coding sequence” refers to that portion of a gene that encodes an amino acid sequence of a protein.

As used herein, the term “sense strand” refers to that strand of a double-stranded nucleic acid molecule that has the sequence of the mRNA that encodes the amino acid sequence encoded by the double-stranded nucleic acid molecule.

As used herein, the term “antisense strand” refers to that strand of a double-stranded nucleic acid molecule that is the complement of the sequence of the mRNA that encodes the amino acid sequence encoded by the double-stranded nucleic acid molecule.

As used herein, production by recombinant means by using recombinant DNA methods means the use of the known methods of molecular biology for expressing proteins encoded by cloned DNA, including cloning expression of genes and methods, such as gene shuffling and phage display with screening for desired specificities.

As used herein, a splice variant refers to a variant produced by differential processing of a primary transcript of genomic DNA that results in more than one type of mRNA.

As used herein, a composition refers to any mixture of two or more products or compounds. It can be a solution, a suspension, liquid, powder, a paste, aqueous, non-aqueous or any combination thereof.

As used herein, a combination refers to any association between two or more items. A combination can be packaged as a kit.

As used herein, “packaging material” refers to a physical structure housing the components (e.g., one or more regulatory regions, reporter constructs containing the regulatory regions or cells into which the reporter constructs have been introduced) of the kit. The packaging material can maintain the components sterilely, and can be made of material and containers commonly used for such purposes (e.g., paper, corrugated fiber, glass, plastic, foil, ampules, vials, tubes and others). The label or packaging insert can include appropriate written instructions, for example, practicing a method provided herein.

As used herein, the “database” means a collection of information, such as information (i.e., sequences) representative of two or more regulatory regions. Databases are typically present on computer readable medium so that they can be accessed and analyzed.

As used herein, the singular forms “a”, “and,” and “the” include plural referents unless the context clearly indicates otherwise. Thus, for example, reference to “a gene regulatory region” includes a plurality of such regulatory regions and reference to “a responder cell” includes reference to one or more such responder cells (e.g., a collection or library of responder cells), and so forth.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

### B. Collections of Cellular Reporter Cells and Assays Using the Collections

Collections of cells, designated responder cells, that contain regulatory regions operatively linked to reporter genes, are provided. The collections, which are generally addressable, are used in cell-based screening assays for drug discovery, target evaluation and other applications are provided. Methods for preparing the collections of cells, including identification of responder genes, and isolation of the regulatory regions, preparation of the cells and methods that use the cells are provided. In particular, as described herein, the methods employ one or more of the following steps and employ or produce the following products:

1) selecting target genomes or subsets thereof and identifying genes with altered expression;

2) identifying genes with altered expression, identifying and isolating gene regulatory regions;

3) preparing reporter gene constructs and selection of vectors

4) introducing the reporter gene constructs into cells, including optionally preparing vectors, and preparing cells; and

5) screening and profiling the resulting collections of cells. Each aspect is discussed in turn below.

Provided herein are addressable collections of cells. At each locus or address the cells contain a particular regulatory region linked to nucleic acid encoding a reporter or linked to nucleic acid such that upon binding and initiation of transcription of the promoter or activation or repression of the regulatory region a detectable signal is produced.

The addressable collection of cells permits assessment of the effects of uncharacterized and characterized perturbations, including effector molecules, and serve as a biosensor for assessing such perturbations. The collections of cells can contain regulatory regions from, for example, a particular organisms, an organism or a tissue or organ thereof.

Also provided are methods for producing the cells, including identification of the regulatory regions, identified regulatory regions, nucleic acid constructs containing the regulatory regions and cells containing constructs that include the regulatory regions.

A goal is to generate a large number of constructs and to create collections of responder cells for a variety of perturbations and/or originating cells types, that express a reporter, such as a luciferase, under the control of the regulatory regions, such as promoters. These collections can be used to screen for compounds, such as for specific disorders and for identification of the cellular or biochemical targets of known or unknown (characterized or uncharacterized) perturbations, such as characterized or uncharacterized small effector molecules and other compounds that are candidates for treatment of a particular disorder or condition.

A strategy in using the cellular collection is to narrow down targets that a test compound or other perturbation modulates with the goal of identifying targets of the compound or perturbation. For example, the collection, such an array of cells on a chip or high density microtiter plate, is exposed to a compound that has a known inhibitory activity. The cells that express altered levels of reporters are identified. Such information, which can be stored in a database or otherwise recorded, such as an image of the collection or a scan of the collection noting the response, provides a “signature” for that particular compound. Other compounds having a similar or identical signature should have the same effects. Also, subcollections of the cells that respond to particular perturbations can be prepared and, for example, can be used to study particular pathways and for cellular target identification.

By narrowing down the identify of affected genes for a particular perturbation, it is possible to test other compounds known to have the same effect as the original compound and by virtue of the results obtained it is possible to identify where in a pathway a particular perturbation, such as a compound, acts. Thus, the cell-based screen serves as a filter to get hits for particular genes in a pathway and to thereby identify the targets of small molecules.

The addressable collections of cells can be adapted for a variety of applications and have uses and applications that go beyond those for which gene chips have been applied. For example:

1) Once the initial profile experiment is performed, the possibility of rapidly re-arraying only the responder populations exists to prepare cellular arrays of populations that respond to characterized (known) perturbations for testing on uncharacterized perturbations.

2) Cellular reporter arrays allow real-time detection of changes in gene expression with an appropriate reporter gene, such as a luciferase or fluorophore, coupled to a detector that can follow the kinetics.

3) Each responding reporter cell line for a given input immediately serves as a reporter gene assay for modulators of the input and derived signals.

4) Compound profile databases can be created and searched for similar profiles. This information can be used to functionally cluster compounds.

5) Profiles for unknown genes can be matched to knowns for gene function identification.

6) Profiles for input mutant or disease genes can be matched to compound profiles to indicate compound mechanism of action.

7) Compounds for a cell-based screening program can categorized by profiles. This data enhances the drug discovery process by providing decision information. For example, if 100 compounds from screening can be grouped into 5 distinct profile patterns, he most chemically tractable compounds from each set can be selected.

8) Multidimensional combinatorial arrays can be achieved where multiple inputs are added to the array in serial or simultaneously. Coupled with automation, higher-density formats and sophisticated imaging, more complex screens can be performed.

9) Cellular reporter array experiments are inexpensive compared to gene chips, given the low cost of cells, reagents and supports.

1. Selecting Target Genomes or Subsets Thereof and Identifying Genes with Altered Expression

A genome of interest or a cell type, such as cells from diseased tissue or a particular or tissue are selected, for identification of responder genes. The cells are exposed to a perturbation of interest or to a plurality of perturbations, and genes with altered expression are identified.

Global gene expression levels are measured by any suitable method to detect induction or repression of genes under selected perturbations. These methods include techniques that employing hybridization of nucleic acid probes coupled with detection of hybrids, such as by fluorescence, radioactivity and molecular weight. The techniques include, but are not limited to, for example, cDNA microarrays, gene chips and differential display methods.

Cells, prokaryotic and eukaryotic, generally animal, plant and microbial cells, such as, but not limited to, mammalian tissue and tissue culture cells, are grown under appropriate perturbations for the particular cell type and exposed, generally for a predetermined time, to a perturbation, such as compound of interest. After treatment, cells are collected such as by pelleting, homogenization or lysis by detergents and total RNA isolated.

For microarray experiments, cDNA can be generated from the mRNA template using reverse transcriptase followed by DNA polymerase. The resulting cDNA is transcribed into cRNA in the presence of detectable ribonucleotides, such as biotinylated ribonucleotides, hybridized to a microarray and scanned by a chip reader, such as a charge coupled device (CCD) coupled to an image reader system and, if needed, appropriate software. Each pixel of the microarray contains probes that correspond to specific genes such that only biotinylated cRNA corresponding to that gene will bind and generate signal. The intensity of the signal from a particular area on the microarray correlates with the relative quantity of a gene's transcript levels from the cells.

The relative presence and identity of all polynucleoides, such as genes, represented on the microarray can be determined or is known. By comparing the treated and untreated cell samples, the magnitude and type of change can be determined for any polynucleotide, such as a gene. From this information, a list of the polynucleotides, such as genes, exhibiting the greatest increase or decrease in expression in response to a substance or a stimulus can be determined. By knowing the identity of these polynucleotides, such as genes, and their sequences, regulatory regions that mediate the increase or decrease in expression in response to a substance or a stimulus can be identified.

For the collections and methods herein, any change in expression of a gene is of interest, and particularly those that exhibit at least a 3-6 fold change, which is usually sufficient to obtain a regulatory region that will give a robust detectable signal. The fold change to select, however, can be determined empirically or selected as desired for particular perturbations and cells, such as from 0.5-fold to 10-fold or more, such as 1 to 8-fold, 2-7-fold, 3 to 8-fold. Exemplary methods to identify, isolate and clone the regulatory regions for these genes are known and some are described herein. EXAMPLE 1 provides an application of this approach for identifying inducibly regulated genes and regulatory regions thereof.

In certain embodiments, as discussed below, gene chips are used to identify genes that are up- or down-regulated in response to a particular perturbation. In some embodiments, all genes that exhibit altered expression in the presence of the perturbation compared to its absence or to another perturbation are isolated and serve as candidates from which regulatory regions are isolated. In other embodiments, a pre-selected number of regulatory regions, such as the top ten, for example, of inducible and/or repressible genes for any given system, are selected. The regulatory regions from the genes are isolated and linked to nucleic acid encoding a suitable or convenient reporter, such as a luciferase. The construct is introduced into a suitable vector, such as a retroviral vector, and introduced into the original cell type to reconstitute the activity(ies) observed in the gene chip experiment. The resulting constructs and cells are used to screen for unknown or uncharacterized perturbations that have a desired effect.

For any selected target system, such as an organism, a tissue in an organism, an organ in an organism and genes involved in a particular pathway, responder genes are identified. The regulatory regions are then identified, linked to reporters and introduced into cells. The resulting collection of cells serves as a sensor for perturbations, including signals, events, small molecule effectors and other compounds and conditions that alter gene expression in the selected targeted collection.

Any method for detecting a change in expression in the presence or absence of a perturbation can be employed. Methods that detect mRNA or cDNA derived therefrom and protein expression are contemplated.

For exemplification, identification of the regulated genes using gene chips is provided herein. It is understood that any region of a genome that alters or otherwise modulates gene expression is contemplated. Furthermore any method for identifying such regulatory regions is contemplated. Gene chips provide a convenient means for identification of regulated genes and facilitate rapid screening of large number of genes for relative changes in expression. Expression analysis including nucleic acid hybridization conditions using gene chips is well known (see, e.g., U.S. Pat. No. 6,040,138). Quantitation of relative amounts of gene expression in order to identify changes in expression is also known (see, e.g., U.S. Pat. No. 6,132,969). Any method for such analyses can be employed.

Many candidate genes and their regulatory regions are screened to identify the responders. For example, to identify one or more genes whose expression changes in response to a drug, gene expression is determined following treatment of a cell, tissue or organ, or a subject with the drug and is compared to gene expression in the absence of the drug. Nucleic acids, generally RNA, from the cells are isolated and are hybridized to an oligonucleotide array of known nucleic acids to identify those whose expression is different in the treated and untreated cells. Changes in expression levels are determined in order to identify responder genes, including robust responders.

2. Identifying Genes with Altered Expression, Identifying and Isolating Gene Regulatory Regions

In general, regulatory regions are isolated or identified for genes whose expression is altered. In some embodiments, any such gene is used as a source of a regulatory region and in other embodiments, those that are altered a predetermined amount more than other genes are selected. Those whose expression is altered substantially, such as at least two or three-fold are referred to herein robust responder regulatory regions. The particular increase depends upon the system of interest and the perturbations under which the system is examined.

Any method for identifying genes with altered expression is contemplated for use herein. In addition, provided herein are methods for detecting changes in expression levels among a plurality of genes to identify responder genes. As noted, genes whose expression is altered in response to a selected perturbation or perturbations(s) are designated as responder genes and their regulatory regions are designated responder regulatory regions.

a. Expression Analysis

Any change in gene expression or manifestation thereof can be measured when identifying responder genes. The selected change in expression can depend upon the system under consideration and the types of genes and perturbations assessed. Many methods for assessing gene expression by measuring or detecting mRNA are known to those of skill in the art. Any such method can be employed herein. Such methods include, but are not limited to, gene chips with oligoncleotides of predetermined substantially unique specificity; dot blots, and other hybridization methods in which RNA produced by cells can be compared.

The methods identify genes whose expression is different in the presence and absence of the perturbation by virtue of hybridization to a particular oligonucleotide or other method. Then, either by sequencing the gene and its flanking regions, typically at least 100, 200, 500, 1000, 2500 or more nucleotides upstream and/or downstream, or using a database, regulatory regions can be identified. For example, many regulatory signals are located in the region including about 2500 bps upstream of the ATG start codon. Using an appropriate program and database or sequence, the region can be identified and isolated or synthesized. For example, the region can be obtained using amplification with appropriate primers, and then operatively linked to a nucleic acid encoding a reporter or inserted into a vector, such as a retroviral vector, containing the nucleic acid encoding the reporter. The vector can be introduced into the same cells (or different cells) from which the responder gene was originally identified and the activity can be reconstituted and observed by virtue of expression of the reporter.

Changes in gene expression that can be measured include changes that occur over time in response to a perturbation, such as a test substance or stimulus or condition, and changes that are transient and changes that have a definable endpoint and/or are permanent. For example, a cell can be exposed to a perturbation, such as treatment with a test substance or stimulus and expression of a plurality of genes determined over a period of minutes, such as, for example (e.g., 0, 15, 30 minute intervals, or less, hours (e.g., 1, 2, 3, 4, 6, 8, 10, 12, 16, 20, 24 hour intervals, or less, or even days (e.g., 1, 2, 3, and more days).

Changes in gene expression also include changes that occur at different doses of test perturbation or the degree of exposure to the perturbation. For example, a cell can be treated with a high, moderate or low concentration of a test substance. A cell can be exposed to high, moderate or low temperature (e.g., 30, 32, 35, 39, 42, 45° C. and higher) or pH (e.g., 6.0, 6.5, 6.8, 7.0, 7.2, 7.8, 8.0, 8.5, and higher or lower) changes. A stimulus, such as increased, decreased or absence (i.e., hypoxia) of oxygen also can be assayed at fine or large deviations from normal oxygen levels.

Changes in gene expression include relative and absolute differences in gene transcript levels, and transient and permanent changes. Relative differences can be determined, for example, by a comparison of hybridization signals obtained in the presence and absence of a test substance or stimulus, or obtained from two or more treatments. Hybridization intensity can be representative of transcript level. Absolute differences can be determined, for example, by inclusion of known concentration(s) of one or more target nucleic acids (e.g. a panel of different concentrations) and comparing the hybridization intensity of unknowns with the known nucleic acid by generation of a standard curve.

1) Preparing Nucleic Acids for Expression Analysis

Nucleic acids that can be used for determining changes in gene expression include RNA, particularly mRNA. Nucleic acid (such as mRNA) can be isolated from cells, tissues or organs or from samples using any known method. For example, to isolate mRNA, an oligo-dT column or beads can be used to purify polyA containing nucleic acid. RNA can be reverse transcribed into DNA using reverse transcriptase followed by DNA polymerase or PCR amplification, then cRNA, if desired, and subsequently used for determining expression levels (see, e.g., Example 1). Labeled cDNA can be prepared from mRNA by oligo dT-primed or random-primed reverse transcription, both of which are well known in the art (see e.g., Klug et al. (1987) Methods Enzymol. 152:316-325). Reverse transcription can be performed in the presence of a dNTP conjugated to a detectable label, such as a fluorescently labeled dNTP. Alternatively, RNA can be present in a sample.

A sample can be a biological sample, such as a tissue or fluid. Samples include, but are not limited to, sputum, blood, blood cells (e.g., white cells), bone marrow cells, tissue or biopsy samples, stool, urine, synovial fluid, sweat, peritoneal fluid, pleural fluid, spinal or cranial fluid or cells therefrom. Samples also can include sections of tissues such as frozen sections taken for histological purposes. Thus, essentially any sample that contains RNA, particularly mRNA or portions thereof, can be used for determining gene expression and, therefore changes in gene expression when the sample has been exposed to (in vivo, ex vivo or in vitro) to a test or known perturbation.

The cells can be obtained from tissues, organs or other biological samples to assess disease progression, to identify pathways in disease progression, and to assess treatment effectiveness, for example. A fingerprint (profile) of the disease or progress thereof can be obtained.

The nucleic acids obtained from a cell, tissue or organ, treated or untreated with (exposed/not exposed to) a perturbation, such as test substance or stimulus, can be labeled before, during, or after hybridization to, for example, a gene chip array, although typically nucleic acids are labeled before hybridization. The labels can be incorporated by any of a number of methods known to those of skill in the art. For example, polymerase chain reaction (PCR) with labeled primers or labeled nucleotides will produce a labeled amplification product. Labels that can be employed include radioisotope labeled nucleotides (e.g., dCTP), fluorescein-labeled nucleotides (UTP or CTP). A label can be attached directly or via a linker to the nucleic acid sample (e.g., mRNA, polyA mRNA, cDNA and PNA) or to the amplification product after the amplification is completed using methods known to those of skill in the art including, for example nick translation or end-labeling, such as with labeled RNA. “Direct labels” are directly attached to or incorporated into the nucleic acid prior to hybridization. Indirect labels are attached to the hybrid duplex after hybridization. For example, an indirect label, such as biotin, can be attached to the nucleic acid prior to the hybridization. Following hybridization, an aviden-conjugated fluorophore will bind the biotin bearing hybrid duplexes to facilitate detection.

2) Identifying Regulatory Regions

Any method for identifying regulatory regions can be employed; it is also contemplated that known regulatory regions can be included among the loci of cells. In one method, provided herein, a gene expression profile of a cell, tissue or organ, or other biological sample from a subject, such as a human, and rodent, such as mouse or other animal, particularly mammals, is obtained in the presence and absence of a substance or other perturbation. These profiles can be obtained using oligonucleotide arrays, including commercially available gene chips, and other high throughput formats. The sample cells or tissues are subjected to the perturbation and mRNA is hybridized to the gene chip and compared to mRNA from untreated cells. The hybridizing nucleic acid molecules in the gene chips serve to identify the genes for which mRNA present or absent in the treated cells, and wose expression is altered in response thereto are identified.

Thus, in one embodiment, oligonucleotide arrays and hybridization analyses are used to identify altered gene transcript levels in response to a test substance or other perturbation. By performing gene-chip studies on cells treated with a test substance or stimulus, genes whose expression pattern changes are identified. Generally genes with a substantial difference in expression, such as 0.5-, 1-, 2-, 3-, 5-, 10- or greater fold alteration, such as an increase or decrease in expression in the presence of the test substance or other perturbation in comparison to the absence of the test substance or other perturbation are identified. Those with a difference of at least about 2- or 3-fold are referred to as robust responder genes.

Candidate regulatory regions, such as promoters, are then identified using available genomic sequence data or other molecular biological techniques or by sequencing of upstream regions. Reporter gene constructs driven by the gene regulatory regions are produced and introduced into cells thereby producing cells containing the reporters (i.e., responder cells) that respond to the substance or stimulus.

For example, public or proprietary (such as the database owned by Celera or Incyte) sequence databases are used to select the regulatory region or at least a portion thereof that mediates the increase or decrease in gene transcript levels in response to the test substance or other perturbation. Candidate regulatory regions, synthetically produced or isolated from genomic DNA by any suitable known biological techniques, such as, for example, polymerase chain reaction of a genomic template with primers that flank the candidate regulatory region, are cloned into a reporter gene expression construct, such as by operatively linking such nucleic acid to nucleic acid encoding a molecule that encodes a reporter, such as a luciferase, β-galactosidase, red, blue or green fluorescent protein, chloramphenicol acetyltransferase and others of the myriad of known reporters. The construct can be introduced into a suitable plasmid or vector, such as a retroviral vector, such as but are not limited to, Moloney murine leukemia virus (MoMLV) and derivatives thereof, such as MFG vectors (see, e.g., U.S. Pat. No. 6316255 B1, ATCC acession No. 68754) and pLJ vectors (see, e.g., Korman et al. (1987) Proc. Natl. Acad. Sci. U.S.A. 84:2150-2154); myeloproliferative sarcoma virus (MPSV); murine embryonic stem cell virus (MESV), murine stem cell virus (MSCV); lentivirus vectors, such as vectors produced from a human immunodeficiency virus (HIV), a simian immunodeficiency virus (SIV), and equine infectious-anaemia virus (EIAV); spleen focus forming virus (SFFV); and the MSCV retroviral expression system (Clontech), which is useful for transformation of embryonic stem cell. The particular vector selected depends upon the cell type and response of interest.

The reporter, under the control of the regulatory region, is introduced into cells, such as biologically interesting cell types, for example neuronal cells, cells from a particular organ or tissue, and cells used in the original gene expression profiling study, to produce cells that respond to the substance or perturbation. The resulting cells are herein referred to as responder cells. Those in which the change in response in the presence of the substance or perturbation is two- to three-fold greater (under the perturbations in which the regions was originally identified) are referred to as robust responder cells.

A plurality, such as a library or collection, of different sets of responder cells, each set of cells containing a reporter driven by a different gene regulatory region, for example in an addressable, such as an arrayed format, are produced. The resulting collection is useful in high-throughput screening assays for expression profiling of test substances or stimuli.

An arrayed format of responder cells (e.g., a responder panel) in a plate, such as a 96, 384, 1536 or higher density well microtiter dish) can be used for expression profiling of a substance or stimulus in living cells. Expression profiling of a perturbations, such as a substance or stimulus or condition or modulator, using regulatory regions of biologically important genes, such as growth promoters (oncogenes) or inhibitors (tumor suppressors), modulators of immune response and developmental regulators, can be used to characterize various perturbations, such as substances and stimuli, for their effects on these particular pathways. The methods provided herein therefore increase the number of reporter assays available for monitoring the effect of a substance or a stimulus and the speed at which they are generated, which is advantageous for meeting the throughput goals of a high-throughput screening operation.

Hence methods for identifying a regulatory region of a gene among a plurality of gene regulatory regions are provided. In one embodiment, a method includes contacting a cell with a test substance or stimulus; determining expression of a plurality of genes in the cell in the presence of the substance or stimulus in comparison to the absence of the substance or stimulus; identifying at least one gene whose expression is increased at least 3-fold in the presence of the substance or stimulus in comparison to the absence of the substance; or identifying at least one gene whose expression is decreased at least 6-fold in the presence of the substance or stimulus in comparison to the absence of the substance; and selecting the regulatory region of the gene that confers increased or decreased expression in response to the test substance or stimulus.

b. Gene Chips for Expression Analyses

Addressable collections of oligonucleotides are used to identify and optionally quantify or determine relative amounts transcripts expressed in the cells. For purposes herein, such addressable collections are exemplified by gene chips, which are arrays of oligonucleotides generally linked to a selected solid support, such as a silicon chip or other inert or derivatized surface. Other addressable collections, such as chemically or electronically labeled oligonucleotides also can be used.

Oligonucleotides can be of any length but typically range in size from a few monomeric units, such three (3) to four (4), to several tens of monomeric units. The length of the oligonucleotide depends upon the system under study; generally oligonucleotides are selected of a complexity that will hybridize to a transcript from one gene only. For example, for the human genome, such length is about 14 to 16 nucleotide bases. If a genome or subset thereof of lower complexity is selected, or if unique hybridization is not desired, shorter oligonucleotides can be used. Exemplary oligonucleotide lengths are from about 5-15 base pairs, 15-25 base pairs, 25-50 base pairs, 75 to 100 base pairs, 100-250 base pairs or longer. Oligonucleotides can be a synthetic oligomer, a full-length cDNA molecule, a less-than full length cDNA, or a subsequence of a gene, optionally including introns.

Gene chip arrays can contain as few as about 25, 50, 100, 250, 500 or 1000 oligonucleotides that are different in one or more nucleotides or 2500, 5000, 10,000, 20,000, 30,000, 40,000, 50,000, 75,000, 100,000, 250,000, 500,000, 1,000,000 or more oligonucleotides that are different in one or more nucleotides. The greater the number of oligonucleotides on the array representing different gene sequences, the more robust responders and their gene regulatory regions can be identified. Thus, oligonucleotides that hybridize to all or almost all genes in an organism's genome are ideal for screening. Such comprehensiveness is not required in order to practice the methods herein. The number of oligonucleotides is a function of the system under study, the desired specificity and the number of responding genes desired. Accordingly, oligonucleotide arrays in which all or a subset of the oligonucleotides represent partial or incomplete genomes can be used, for example 10-20%, 20-30%, 30-40%, 50-60%, 60-75%, or 75-85%, or more (e.g., 90% or 95%)

Gene chip arrays can have any oligonucleotide density; the greater the density the greater the number of oligonucleotides that can be screened on a given chip size. Density can be as few as 1-10, such as 1 2, 4, 5, 6, 8 and 10) oligonucleotides per cm². Density can be as many as 10-100, such as 10-15, 15-20, 20-30, 30-40, 40-50, 50-60, 60-70, 70-80 and 90-100, oligonucleotides per cm² or more. Greater density arrays can afford economies of scale. High density chips are commercially available (i.e. from Affymetrix).

The substrate to which the oligonucleotides are attached include any impermeable or semi-permeable, rigid or semi-rigid, substance substantially inert so as not to interfere with the use of the chip in hybridization reactions. The substrate can be a contiguous two-dimensional surface or can be perforated, for example. Exemplary substrates compatible with hybridization reactions include, but are not limited to, inorganics, natural polymers, and synthetic polymers. These include, for example: cellulose, nitrocellulose, glass, silica gels, glass, coated and derivatized glass, plastics, such as polypropylene, polystyrene, polystyrene cross-linked with divinylbenzene or other such cross-linking agent (se, e.g., Merrifield (1964) Biochemistry 3:1385-1390), polyacrylamides, latex gels, polystyrene, dextran, polyacrylamides, rubber, silicon, plastics, nitrocellulose, celluloses, natural sponges, and many others. The substrate matrices are typically insoluble substrates that are solid, porous, deformable, or hard, and have any required structure and geometry, including, but not limited to: beads, pellets, disks, capillaries, hollow fibers, needles, solid fibers, random shapes, thin films and membranes.

For example, in order to rapidly identify a gene whose expression is increased or decreased each oligonucleotide or a subset of the oligonucleotides of the addressable collection, such as an array on a solid support, can represent a known gene or a gene polymorphism, mutant or truncated or deleted form of a gene or combinations thereof. Transcripts or nucleic acid derived from transcripts, such as RNA or cDNA derived from the RNA, of a cell subjected to a treatment, such as contacting with a test substance or other signal, to the oligonucleotides are hybridized to the gene chip.

In addition the amount of RNA from a cell or nucleic acid derived from RNA of a cell that hybridizes to oligonucleotides of the array can reflect the level of the mRNA transcript in the cell. By labeling the RNA from a cell or nucleic acid derived from RNA, and comparing the intensity of the signal given by the label following hybridization to oligonucleotides of the array, relative or absolute amounts of gene transcript are quantified. Any differences in transcript levels in the presence and absence of the test perturbation are revealed.

Since each locus in the addressable array of oligonucleotides is known, the identity of hybridizing nucleic acid is then determined and the genes identified. Such genes are responder genes. The oligonucleotides of the chip, or at least a subset of oligonucleotides, are known a priori to hybridize specifically with particular genes. By knowing the position of each oligonucleotide on the array and the gene to which the oligonucleotide hybridizes, determining the position on the array that gives a hybridization signal identifies the gene whose expression is altered. Alternatively if the specificity of the set of oligonucleotides is not known, the transcripts that exhibit altered expression can be sequenced and the genes identified.

In an initial screen for responder genes, the genes are selected based upon the amount of change in expression in response to a perturbation, such as a test substance or stimulus. A gene is selected when it exhibits altered, such as increased or decreased, expression compared to other genes or to the control in the absence of the perturbation. For those with increased expression, responders can have any fold-increase, such as one, two, three, four, five, or more-fold than other genes or the control. Generally a gene is selected when it exhibits increased expression that places the gene among a predetermined number, such as the top 100, 50, 20, 5 or 2 genes whose expression is increased among the plurality of genes. In yet another embodiment, the gene is selected when it exhibits increased expression greater than increased expression of any other gene among the plurality of genes. In other embodiments, the gene is selected when it exhibits three-fold, six-fold, 10-fold, 15-fold, 20-fold, 25-fold, 50-fold or greater expression (relative or absolute) in the presence of the perturbation test substance or stimulus as compared to the absence of the test substance or stimulus. The particular increase desired or needed can be empirically determined for the particular system under study.

For those with decreased expression, a gene is selected when its expression is decreased to a greater extent than decreased expression of a selected number, such as the top 100, 50, 20, 5 or 2 genes whose expression is less than other genes. In other embodiments, a gene is selected when its expression is decreased to the extent that it is among the top 10, 5 or 2 genes whose expression is decreased among the plurality of genes. In still further embodiments, a gene is selected when its expression is decreased to a greater extent than decreased expression of any other gene among the plurality of genes. In yet additional embodiments, the gene is selected when it exhibits three-fold, six-fold, 10-fold, 15-fold, 20-fold, 25-fold, 50-fold or less expression (relative or absolute) in the presence of the test substance or stimulus as compared to the absence of the test substance or stimulus.

Hybridizing transcripts also identify which, if any among the plurality of genes exhibits increased, such as two- or three-fold or more or decreased, such as six-fold or more, transcript levels in the presence of the test perturbation, such as a substance or stimulus, in comparison to the absence of the test substance or stimulus.

Exemplary conditions for gene chip hybridization include low stringency, in 6×SSPE-T at 37° C. (0.005% Triton X-100) hybridization followed by washes at a higher stringency (e.g., 1×SSPE-T at 37° C.) to reduce mismatched hybrids. Washes can be performed at increasing stringency (e.g., as low as 0.25×SSPE-T at 37° C. to 50° C.) until a desired level of specificity is obtained. Hybridization specificity can be evaluated by comparison of hybridization to the test probes with hybridization to the various controls that can be present (e.g., expression level control, normalization control and mismatch controls).

Additional examples of hybridization conditions useful for gene chip and traditional nucleic acid hybridization (e.g., northerns and southern blots) are, for moderately stringent hybridization conditions: 2×SSC/0.1% SDS at about 37° C. or 42° C. (hybridization); 0.5×SSC/0.1% SDS at about room temperature (low stringency wash); 0.5×SSC/0.1% SDS at about 42° C. (moderate stringency wash); for moderately-high stringency hybridization conditions: 2×SSC/0.1% SDS at about 37° C. or 42° C. (hybridization); 0.5×SSC/0.1% SDS at about room temperature (low stringency wash); 0.5×SSC/0.1% SDS at about 42° C. (moderate stringency wash); and 0.1 ×SSC/0.1% SDS at about 52° C. (moderately-high stringency wash); for high stringency hybridization conditions: 2×SSC/0.1% SDS at about 37° C. or 42° C. (hybridization); 0.5×SSC/0.1% SDS at about room temperature (low stringency wash); 0.5×SSC/0.1% SDS at about 42° C. (moderate stringency wash); and 0.1×SSC/0.1% SDS at about 65° C. (high stringency wash).

Hybridization signals can vary in strength according to hybridization efficiency, the amount of label on the nucleic acid and the amount of the particular nucleic acid in the sample. Typically nucleic acids present at very low levels (e.g., <1 pM) will show a very weak signal. A threshold intensity can be selected below which a signal is not counted as being essentially indistinguishable from background. In any case, it is the difference in gene expression (test substance or stimulus, treated vs. untreated) that determines the genes for subsequent selection of their regulatory region. Thus, extremely low levels of detection sensitivity are not required in order to practice methods provided herein.

Detecting nucleic acids hybridized to oligonucleotides of the array depends on the nature of the detectable label. Thus, for example, where a colorimetric label is used, the label can be visualized. Where a radioactive labeled nucleic acid is used, the radiation can be detected (e.g with photographic film or a solid state counter). Nucleic acids labeled with a fluorescent label and detection of the label on the oligonucleotide array is typically accomplished with a fluorescent microscope. The hybridized array is excited with a light source at the appropriate excitation wavelength and the resulting fluorescence emission detected which reflects the quantity of hybridized transcript. In this particular example, quantitation is facilitated by the use of a fluorescence microscope which can be equipped with an automated stage for automatic scanning of the hybridized array. Thus, in the simplest form of gene expression analysis using an oligonucleotide array, quantitation of gene transcripts is determined by measuring and comparing the intensity of the label (e.g., fluorescence) at each oligonucleotide position on the array following hybridization of treated and hybridization of untreated samples.

Nucleic acid from cells treated and untreated with a test compound or stimulus can be individually or simultaneously hybridized to an array. In the case of simultaneous hybridization, the nucleic acid of each sample will be differentially labeled to facilitate distinguishing the amounts of gene transcripts from each sample. For example, using green and red fluorophores, the cDNA from the treated cell sample can fluoresce green and the cDNA from the untreated cell sample can fluoresce red when the fluorophores are excited. If treatment has no effect on the expression of a particular gene, transcript levels will be equal in both cell samples and, upon reverse transcription, red and green fluorescently labeled cDNA will be equal. Thus, when hybridized to the oligonucleotide of the array, the hybridized nucleic acid will emit wavelengths characteristic of green and red fluorophores in equal amounts. In contrast, when a cell is treated with test substance or stimulus that, directly or indirectly, increases the mRNA in the cell, the amount of green to red fluorescence will increase. When the test substance or stimulus decreases the mRNA prevalence, the green to red ratio will decrease.

The use of two-color fluorescence labeling and detection to measure changes in gene expression can be used (see, e.g., Shena et al. (1995) Science 270:467). Simultaneously analyzing cDNA labeled with two different labels (e.g., fluorophores) provides a direct and internally controlled comparison of the mRNA levels corresponding to each arrayed oligonucleotide; variations from minor differences in experimental conditions, such as hybridization conditions, do not affect the analyses.

Thus, the method provided herein can include: hybridizing to two different oligonucleotide arrays a labeled mRNA or nucleic acid derived therefrom, where each label is the same,; hybridizing a labeled mRNA or nucleic acid derived therefrom simultaneously to an oligonucleotide array, where each label is different; and hybridizing labeled mRNA or nucleic acid derived therefrom sequentially to an oligonucleotide array, wherein each label is the same or different.

1) Oligonucleotide Controls

Gene chip arrays can include one or more oligonucleotides for mismatch control, expression level control or for normalization control. For example, each oligonucleotide of the array that represents a known gene, that is, it specifically hybridizes to a gene transcript or nucleic acid produced from a transcript, can have a mismatch control oligonucleotide. The mismatch can include one or more mismatched bases. The mismatch(s) can be located at or near the center of the probe such that the mismatch is most likely to destabilize the duplex with the target sequence under hybridization conditions, but can be located anywhere, for example, a terminal mismatch. The mismatch control typically has a corresponding test probe that is perfectly complementary to the same particular target sequence.

Mismatches are selected such that under appropriate hybridization conditions the test or control oligonucleotide hybridizes with its target sequence, but the mismatch oligonucleotide does not. Mismatch oligonucleotides therefore indicate whether hybridization is specific or not. For example, if the target gene is present the perfect match oligonucleotide should be consistently brighter than the mismatch oligonucleotide.

When mismatch controls are present, the quantifying step can include calculating the difference in hybridization signal intensity between each of the oligonucleotides and its corresponding mismatch control oligonucleotide. The quantifying can further include calculating the average difference in hybridization signal intensity between each of the oligonucleotides and its corresponding mismatch control oligonucleotide for each gene.

Expression level controls are, for example, oligonucleotides that hybridize to constitutively expressed genes. Expression level controls are typically designed to control for cell health. Covariance of an expression level control with the expression of a target gene indicates whether measured changes in expression level of a gene is due to changes in transcription rate of that gene or to general variations in health of the cell. For example, when a cell is in poor health or lacking a critical metabolite the expression levels of an active target gene and a constitutively expressed gene are expected to decrease. Thus, where the expression levels of an expression level control and the target gene appear to decrease or to increase, the change can be attributed to changes in the metabolic activity of the cell, not to differential expression of the target gene. Virtually any constitutively expressed gene is a suitable target for expression level controls. Typically expression level control genes are “housekeeping genes” including, but not limited to β-actin gene, transferrin receptor and GAPDH.

Normalization controls are often unnecessary for quantitation of a hybridization signal where optimal oligonucleotides that hybridize to particular genes have already been identified. Thus, the hybridization signal produced by an optimal oligonucleotide provides an accurate measure of the concentration of hybridized nucleic acid.

Nevertheless, relative differences in gene expression can be detected without the use of such control oligonucleotides. Therefore, the inclusion of control oligonucleotides is optional.

2) Synthesis of Gene Chips

The oligonucleotides can be synthesized directly on the array by sequentially adding nucleotides to a particular position on the array until the desired oligonucleotide sequence or length is achieved. Alternatively, the oligonucleotides can first be synthesized and then attached on the array. In either case, the sequence and position (i.e., address) of all or a subset of the oligonucleotides on the array will typically be known. The array produced can be redundant with several oligonucleotide molecules representing a particular gene.

Gene chip arrays containing thousands of oligonucleotides complementary to gene sequences, at defined locations on a substrate are known (see, e.g., International PCT application No. WO 90/15070 and can be made by a variety of techniques known in the art including photolithography (see, e.g., Fodor et al. (1991) Science 251:767; Pease et al. (1994)Proc. Natl. Acad. Sci. U.S.A. 91:5022; Lockhart et al.(1996) Nature Biotech 14:1675; and U.S. Pat. Nos. 5,578,832; 5,556,752; and 5,510,270).

A variety of methods are known. For example methods for rapid synthesis and deposition of defined oligonucleotides are also known (see, e.g., Blanchard et al. (1996) Biosensors & Bioelectronics 11:6876); . as are light-directed chemical coupling, and mechanically directed coupling methods (see, e.g., U.S. Pat. No. 5,143,854 and International PCT application Nos. WO 92/10092 and WO 93/09668, which describe methods for forming vast arrays of oligonucleotides, peptides and other biomolecules, referred to as VLSIPS™ procedures (see, also U.S. Pat. No. 6,040,138). U.S. Pat. No. 5,677,195 describes forming oligonucleotides or peptides having diverse sequences on a single substrate by delivering various monomers or other reactants to multiple reaction sites on a single substrate where they are reacted in parallel. A series of channels, grooves, or spots are formed on or adjacent and reagents are selectively flowed through or deposited in the channels, grooves, or spots, forming the array on the substrate. The aforementioned techniques describe synthesis of oligonucleotides directly on the surface of the array, such as a derivatized glass slide. Arrays also can be made by first synthesizing the oligonucleotide and then attaching it to the surface of the substrate e.g., using N-phosphonate or phosphoramidite chemistries (see, e.g., Froehler et al. (1986) Nucleic Acid Res 14:5399; and McBride et al. (1983) Tetrahedron Lett. 24:245). Any type of array, for example, dot blots on a nylon hybridization membrane (see, e.g., Sambrook et al. (1989) Molecular Cloning: A Laboratory Manual (2nd Ed.), Vol. 1-3, Cold Spring Harbor Laboratory, Cold Spring Harbor, N.Y.) can be used.

3) Gene Chip Signal Detection

As discussed, fluorescence emission of transcripts hybridized to oligonucleotides of an array can be detected by scanning confocal laser microscopy. Using the excitation line appropriate for the fluorophore, or for two fluorophores if used, will produce an emission signal whose intensity correlates with the amount of hybridized transcript. Alternatively, a laser that allows simultaneous specimen illumination at wavelengths specific to the two fluorophores and emissions from the two fluorophores can be used for simultaneously analyzing both (see, e.g., Schena et al. (1996) Genome Research 6:639).

In any case, hybridized arrays can be scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Alternatively, other fiber-optic bundles (see, e.g., Ferguson et al. (1996) Nature Biotech. 14:1681 can be used to monitor mRNA levels simultaneously. For any particular hybridization site on the array, a ratio of the emission of the two fluorophores can be calculated. The ratio is independent of the absolute expression level of the gene, but is useful for identifying responder genes whose expression is significantly increased or decreased in response to a perturbation, such as a test substance or stimulus.

C. Exemplary Alternatives to Gene Chip for Expression Analyses

1) Target Arrays

As an alternative, for example, nucleic acid isolated from the cells or other samples and sources can be linked to a solid support, and collections of probes or oligonucleotides of known sequences hybridized thereto. The probes or oligonucleotides can be uniquely labeled, such as by chemical or electronic labeling or by linkage to a detectable tag, such as a colored bead. The expressed genes from cells exposed to a test perturbation are compared to those from a control that is not exposed to the perturbation. Those that are differentially expressed are identified.

2) Other Non-gene Chip Methods for Detecting Changes in Gene Expression

In addition to using gene chips to detect changes in gene expression, changes in gene expression also can be detected by other methods known in the art. For example, differentially expressed genes can be identified by probe hybridization to filters (Palazzolo et al. (1989) Neuron 3:527); Tavtigian et al. (1994) Mol Biol Cell 5:375). Phage and plasmid DNA libraries, such as cDNA libraries, plated at high density on duplicate filters are screened independently with cDNA prepared from treated or untreated cells. The signal intensities of the various individual clones are compared between the two filter sets to determine which clones hybridize preferentially to cDNA obtained from cells treated with a test substance or stimulus in comparison to untreated cells. The clones are isolated and the genes they encode are identified using well established molecular biological techniques.

Another alternative involves the screening of cDNA libraries following subtracting mRNA populations from untreated and cells treated with a test substance or stimulus (see, e.g., Hedrick et al. (1984) Nature 308:149). The method is closely related to differential hybridization described above, but the cDNA library is prepared to favor clones from one mRNA sample over another. The subtracted library generated is depleted for sequences that are shared between the two sources of mRNA, and enriched for those that are present in either treated or untreated samples. Clones from the subtracted library can be characterized directly. Alternatively, they can be screened by a subtracted cDNA probe, or on duplicate filters using two different probes as above.

Another alternative uses differential display of mRNA (see, e.g., Liang et al. (1995) Methods Enzymol 254:304). PCR primers are used to amplify sequences from two mRNA samples by reverse transcription, followed by PCR. The products of these amplification reactions are run side by side, i.e., pairs of lanes contain the same primers but mRNA samples obtained from treated and untreated cells on DNA sequencing gels. Differences in the extent of amplification can be detected by any suitable method, including by eye. Bands that appear to be differentially amplified between the two samples can be excised from the gel and characterized. If the collection of primers is large enough it is possible to identify numerous gene differentially amplified in treated versus untreated cell samples.

Another alternative designated representational Difference Analysis (RDA) of nucleic acid populations from different samples (see, e.g., Lisitsyn et al. (1995) Methods Enzymol. 254:304) can be used. RDA uses PCR to amplify fragments that are not shared between two samples. A hybridization step is followed by restriction digests to remove fragments that are shared from participation as templates in amplification. An amplification step allows retrieval of fragments that are present in higher amounts in one sample compared to the other (i.e., treated vs. untreated cells).

3) Detection of Proteins to Assess Gene Expression

Changes in gene expression also can be detected by changes in the levels of proteins expressed. Any method known to those of skill in the art for assessing protein expression and relative expression, such as antibody arrays that are specific for particular proteins and two-dimensional gel analyses, can be employed. Protein levels can be detected, for example, by enzyme linked immunosorbent assays (ELISAs), immunoprecipitations, immunofluorescence, enzyme immunoassay (EIA), radioimmunoassay (RIA), and Western blot analysis.

An array of antibodies can be used to detect changes in the level of proteins. Biosensors that bind to large numbers of proteins and allow quantitation of protein amounts in a sample (see, e.g., U.S. Pat. No. 5,567,301, which describes a biosensor that includes a substrate material, such as a silicon chip, with antibody immobilized thereon, and an impedance detector for measuring impedance of the antibody are can be employed. Antigen-antibody binding is measured by measuring the impedance of the antigen bound antibody in comparison to unbound antibody.

A biosensor array that binds to proteins are used to detect changes in protein levels in response to a perturbation, such as a test substance or stimulus. For example, U.S. Pat. No. 6,123,819 describes a protein sensor array capable of distinguishing between different molecular structures in a mixture. The device includes a substrate on which nanoscale binding sites in the form of multiple electrode clusters are fabricated in which each binding site includes nanometer scale points extending above the surface of a substrate. These points provide a three-dimensional electrochemical binding profile which mimics a chemical binding site and has selective affinity for a complementary binding site on a target molecule or for the target molecule itself.

3. Preparing Reporter Gene Constructs and Selection of Vectors

a. Isolation of Regulatory Regions

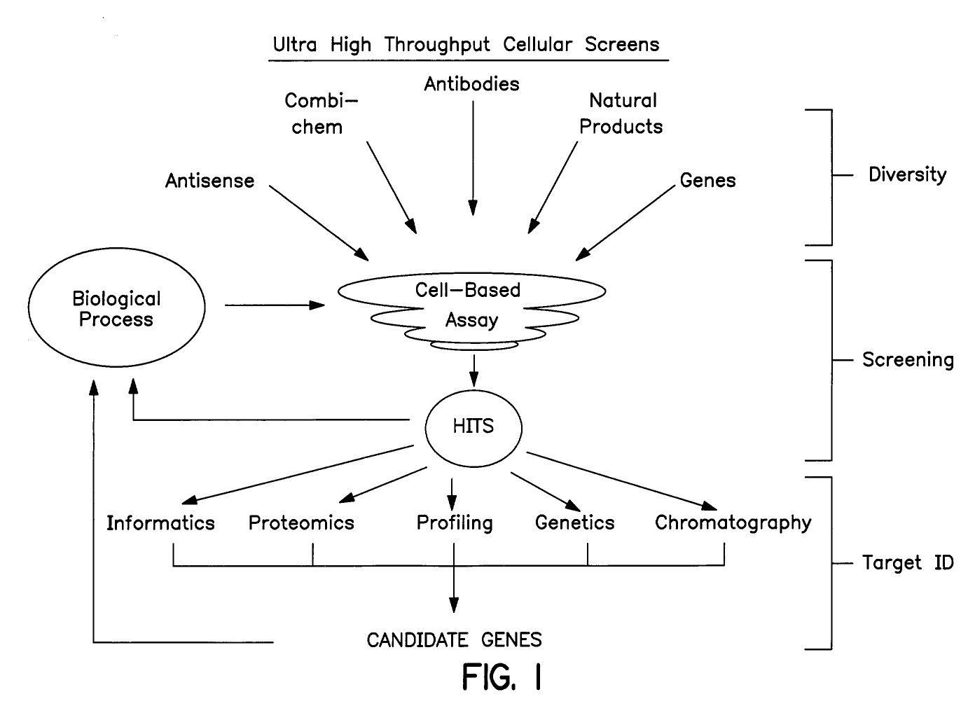

Regulatory regions, such as promoters, for all genes or any subset of genes in a genome are identified, isolated, linked to reporter genes and introduced into cells, such as by insertion into a vector that can infect, transfect or transduce selected cells. A plurality of such regions can be simultaneously identified. The regulatory region is identified and isolated by standard molecular biology techniques, and cloned into reporter constructs. The reporter constructs then can be then addressably arrayed, such as in high-density microtiter plates or on any other suitable support, and introduced in parallel into cells, also in an addressable array, such as a high density microtiter plate, to produce a plethora of distinct reporter cells that can be used in screening assays to identify targets and for drug screening. The cells can be transiently transfected or the cells can be selected for stable expression of the reporter construct if desired as a continuous source of cells for reporters cell assays. A resulting collection of cellular reporter cells is treated with an input perturbation, such as a compound, protein, antibody, expressed cDNA, oligonucleotide or subjected to any desired perturbation, optionally using laboratory automation, and assessed for the effects of that input on cellular reporter genes using appropriate detection device(s). Each input will produce a unique reporter “fingerprint” so that each collection can be used to profile perturbations, such as a compound, protein, antibody, expressed cDNA, oligonucleotide and any other perturbation, in real time. The process is outlined in FIG. 1.

Identification of Inducibly Regulated Promoters

Regulatory elements that control transcription of a gene include the promoter region for the gene. Promoter regions and other transcriptional regulatory regions are usually 5′ or upstream of the gene's coding sequence. The typical eukaryotic promoter includes a transcription initiation site, a binding site (TATA box), initiator, minimal or core promoter, proximal promoter region, and sometimes enhancer, silencer or locus control regions. Normally, sequences 1 to 10 kilobases (kB) upstream of the genes transcriptional start site contain all regulatory regions. Hence, upon identification of an inducible gene, selection of the region about 1 to 10 kB upstream thereof will contain regulatory regions of interest herein.

Identification of an inducible gene by methods herein or other such method permits identification of such regions. These regions can be identified by cloning and sequencing if necessary, and generally by searching public or proprietary databases for sequences identical to the gene of interest. Upon identification of the gene, the 5′ start site (methionine) of the gene and about 10 kB pair sequence upstream is identified. This 10 kB sequence generally contains a promoter region controlling expression of the gene of interest. This analysis is enhanced by searching for consensus promoter regions, or transcription factor binding motif sequences or enhancer elements.

Based upon the identity of the responder gene, the regulatory region is then identified. Identification of candidate regulatory region, such as a promoter-containing region, for any gene can be done by any method known to those of skill in the art, including manually and/or by database searching. For example, following identification of a gene whose expression increases or decreases in the presence of a test substance or stimulus, a regulatory region of the gene can be identified by probing genomic sequences, such as a genomic library) with the gene or fragment thereof for hybridizing sequences that also include 5′ or 3′ untranslated sequences of the gene.

Alternatively, RNA extension (to identify the transcriptional start site) followed by genomic DNA “primer walking” to identify sequences upstream of the transcription start site can be used. These methods are standard and well known in the art (see, e.g., Sambrook et al. (2001) Molecular Cloning: A Laboratory Manual (3rd Ed.), Vol. 1-3, Cold Spring Harbor Laboratory, Cold Spring Harbor, N.Y.).

Candidate gene regulatory regions can be identified by comparison of the gene to a sequence database available in the art now or in the future. For example, a public or proprietary sequence database that includes genomic sequence information can be used to identify sequences located 5′ or 3′ of the translation initiation site of the selected gene, as well as intron(s). Because sequences located 5′ and extending upstream of the translation initiation site frequently contain gene regulatory sequences, nucleotide sequences positioned 5′ of the translation initiation site are good candidates for regulatory sequences and can be selected for cloning into a reporter construct. For example, a sequence that includes the 5′ translation start site (methionine) of the gene and 10 Kb or more upstream of the site contains intronic and exonic portions of the gene, but likely also the promoter region controlling expression of the gene. The embodiment of database searching for selecting candidate gene regulatory regions is exemplified in Example 3.

Sequence databases of any organism can be searched in order to identify candidate regulatory regions. Partial and complete sequence databases of many organisms, including mammals, are available in the art. Databases are available and can be found using any suitable internet search engine to identify sites posting such databases (see, e.g., www.ncbi.nlm.nih.gov/genome/seq/page.cgi?F=HsBlast.html&&ORG=Hs for a human database. Other human databases are available for a fee, such as the database owned by Celera, Inc. Similarly, mouse partial genomic sequences are available (see, e.g., http://www.ncbi.nlm.nih.gov/genome/seq/MmHome.html). The complete yeast Saccharomyces cerevisiae genomic sequence is available (see, e.g., http://www.ncbi.nlm.nih.gov/cgi-bin/Entrez/mapOO?taxid=4932). In addition, the complete Drosophila melanogaster and C. elegans genomic databases are known in the art (see, e.g., http://www.ncbi.nlm.nih.gov/PMGifs/Genomes/7227.html and http://www.ncbi.nlm.nih.gov/cgi-bin/Entrez/mapOO?taxid=6239). Plant databases include, for example, the complete sequence of Arabidopis thaliana (see, e.g., http://www.ncbi.nlm.nih.gov/cgi-bin /Entrez/map_search?chr=arabid.inf). As noted, it is understood that URLs for the databases can change and particular information on the internet can come and go, but equivalent information can be found by searching the internet.

Sequence database analysis can be augmented, if desired or needed, by searching for consensus promoter regions, transcription factor binding sequences or enhancer elements. For example, inspecting a gene for a candidate regulatory region can reveal a known regulatory region or a sequence having significant similarity with a known regulatory region. Thus, including a search for one or more sequences homologous or having significant similarity to a known promoter, transcription factor binding site or enhancer can reveal the presence and location of such sequences in the genomic sequence which can then be cloned into the reporter expression construct. Thus, methods herein can be modified to include the strep of identifying regulatory regions by comparison to other regulatory region sequences, such as known regulatory region sequences, including, but not limited to sequences including promoters, transcription factor binding sites, enhancers, scaffold attachment regions and other such transcription and/or translational regulatory regions.

Candidate regulatory regions can be of any length so long as expression in response to the test substance or stimulus is at least in part reflective of expression in the original screen. In other words, expression of a reporter driven by the selected regulatory region need not precisely mirror expression of the endogenous gene in response to the substance or stimulus. In any event, significant variation between endogenous gene expression and reporter gene expression can be minimized by including larger portions of the candidate regulatory region sequence in the reporter construct. Thus, when first choosing a sequence of a candidate regulatory region for cloning into a reporter, larger sequences can be selected. Candidate regulatory regions can therefore include large sequences such as 10,000-15,000 nucleotides or more, 5000-10,000 nucleotides, 1000-5000 nucleotides, and 50-5000 nucleotides.

Inspecting a gene for consensus promoters, transcription factor binding sites, enhancers and other sequences can reveal the presence of one or more such sequences or a sequence that exhibits significant sequence homology to a consensus sequence. When such a consensus sequence is present, a smaller region of the candidate regulatory region that includes the consensus sequence can be chosen for subsequent cloning into a reporter construct. Of course, should there be multiple consensus sequences in the candidate cis-acting regulatory region of a gene, a sequence can be chosen that includes two or more of the multiple consensus sequences. Candidate regulatory regions can therefore include smaller sequences, for example, 50-5000 nucleotides, such as about 5-10, 10-25, 25-50, 50-75, 75-100, 100-250, 250-500, 1000-2500, or 2500-5000 nucleotides.

The untranslated region/candidate regulatory region can subsequently be cloned into a reporter expression construct and introduced into cells. Expression of the reporter in the presence and absence of the test substance or stimulus confirms that the cloned region contains all or at least a part of the regulatory region that mediates the response to the test substance or stimulus. They can also be used for expression of heterologous proteins.

Repeating the steps of identifying or selecting responder genes and cloning a regulatory region therefrom operatively linked to a reporter produces collections of gene regulatory region-reporter constructs (i.e., a library). The accumulation of collections of gene regulatory regions, and reporter constructs containing gene regulatory regions of the entire complement of an organism (e.g., human gene promoters) would be a highly useful resource.

Methods of producing a plurality of gene regulatory regions, such as a library, compositions containing the gene regulatory regions produced by the methods, as well as methods of producing a plurality of gene regulatory region-reporter constructs and compositions containing a plurality of gene regulatory region-reporter constructs produced by the methods. In one embodiment, the plurality contains gene regulatory region-reporter constructs in which expression of the reporter is increased at least three-fold in the presence of the test substance or stimulus in comparison to the absence of the test substance or stimulus. In another embodiment, the plurality contains gene regulatory region-reporter constructs in which expression of the reporter is decreased at least six-fold in the presence of the test substance or stimulus in comparison to the absence of the test substance or stimulus.

Extraction and Cloning of Regulatory Regions, Such as Promoters

The following methodology was used to extract promoter regions from a sequence database and can be generally applied to any DNA sequence database: Unigene, downloaded from NCBI, was parsed for entries where the coding region is explicitly defined (currently 18289 such entries exist). Three hundred bases from the 5′ end of each coding region are assembled into a FASTA file. This file is then aligned to genomic sequence using the BLAST algorithm. The target genomic database can be NR or HTGS from NCBI, or the Celera genome assembly. The BLAST alignments are parsed to determine the location of the gene in a larger genomic contig, and up to 10 kb of sequence is taken upstream of the translational start site. Several 1000 promoter sequences have been assembled in silico using this technique.

Genomic DNA is prepared from Human 293 cells using DNAzol. Oligonucleotide primers are synthesized from 20, two kB promoter sequences at a time. Polymerase chain reaction (PCR) is used to amplify promoter sequences from chromosomal DNA templates and cloned into standard reporter gene constructs in which the cloned promoter drivers expression of the Firefly Luciferase (luc) gene or some other reporter gene. The DNA encoding each promoter reporter construct is individually amplified in bacterial cells and purified in micro-titer plates using a RevPrep (Molecular Machines) or Qiagen 9600 (Qiagen). Ninety-six well plates of reporter constructs are re-racked into 384-well plates for subsequent use such that each 384-well plate has 4 wells of each reporter construct.

Regulatory regions can be identified by their presence 5′ from a translation initiation site of the gene, within or a part of the gene coding sequence (e.g., within exons), within or be a part of non-coding intragenic sequences (e.g., introns) or located 3′ of the translation stop site. Candidate regulatory regions can therefore be located throughout a genomic sequence, including sequences within 25 bases, 50 bases, 100 bases, 250 bases, 500 bases, 1 Kb, 2 Kb, 3 Kb, 4 Kb, 5 Kb, 7 Kb, 10 Kb, 15 Kb or more from the translation initiation site and translation termination site of a gene. Hence the location of the gene regulatory region relative to the gene coding sequence is not fixed.

For example, a sequence located 5′ of the translation start site can be cloned into the reporter construct. Longer sequence segments of the candidate regulatory region (e.g., 30 Kb, 20 Kb, 10 Kb, or 5 Kb) can first be examined for conferring increased or decreased reporter expression. Smaller segments can then be examined, if desired, in order to identify smaller segments that confer regulation. A segment of the genomic sequence is cloned (using polymerase chain reaction, conventional restriction enzyme cloning or chemical synthesis) into a reporter construct so that reporter expression is controlled by the segment.

Thus, in one embodiment, a regulatory region is located 5′ of the gene coding region and extends upstream of the translation initiation site. The regulatory region can include a promoter or enhancer and can be located in or as part of one or more exons, one or more introns or 3′ of the gene coding region and extending downstream of the translation termination site. In particular aspects, the sequence region extends from about 25, 50, 75, 100, 250, 500, 1000, 2500, 5000, 7500 or 10,000 or more nucleotides upstream of the translation initiation site of the selected gene. In particular additional aspects, the sequence region extends from about 25, 50, 75, 100, 250, 500, 1000, 2500, 5000, 7500 or 10,000 or more nucleotides downstream of the translation termination site of the selected gene.

b. Reporters and Reporter Gene Constructs

Following selection of a regulatory region, based on examination or cloning of genomic sequence with or without inspecting for the presence of consensus regulatory regions or sequences with similarity to such regions (e.g., promoter sequences, transcription factors binding sequences, enhancer sequences, silencers and others), the sequence can be cloned into a reporter expression construct. Operatively linking a sequence including a 5′ untranslated region upstream of the translation initiation site or any other candidate regulatory region of the selected gene to a reporter gene and determining reporter expression in the presence of the test substance or stimulus confirms that the sequence mediates the response to the test substance or stimulus. Additionally, a plurality of these regulatory regions and portions thereof, such a combinations of identified enhancers or protein binding regions, can be operatively to produce constructs with different sensitivities, activities and specificities.

Reporter gene constructs include a reporter gene such as the nucleic acid encoding firefly luciferase, Renilla luciferase, betagalactosidase, green fluorescent protein, secreted alkaline phosphatase, chloramphenicol acetyltransferase or other element under the control of a response-element such as a promoter sequence from the robust responder gene. Reporter moieties also include, for example, fluorescent proteins, such as red, blue and green fluorescent proteins (see, e.g., U.S. Pat. No. 6,232,107, which provides GFPs from Renilla species and other species), the lacZ gene from E. coli, alkaline phosphatase, chloramphenicol acetyltransferase (CAT) and other such well-known reporters.

C. Vectors and Generation of Viral Particles and Reporter (Responder) Cells Containing the Reporter Gene Constructs

The promoters can be inserted into any suitable expression vector, including viral vectors, such as retroviral vectors and other virally-derived vectors, such as AAV, adenovirus vectors, herpes virus vectors, vaccinia virus, lentivirus vectors and other vectors for expression in selected host cells. The vector is selected to have a host range that encompasses the cells of interest. For exemplification herein reference is made to using retroviral constructs, but it is understood that other vector constructs are contemplated.

Vectors are capable of transporting another nucleic acid to which it has been linked into a cell and include plasmids, cosmids or vectors of virus origin. A vector that will remain episomal contains at least an origin of replication for propagation in a cell; other vectors, such as retroviral vectors integrate into a host cell chromosome. Cloning vectors are typically used to genetically manipulate gene sequences while expression vectors are used to express the linked nucleic acid in a cell in vitro, ex vivo or in vivo.

An “expression vector” can contain an origin of replication for propagation in a cell and includes a control element so that expression of a gene operatively linked thereto is influenced by the control element. Control elements include gene regulatory regions (e.g., promoters, transcription factor binding sites and enhancer elements) as set forth herein, that facilitate or direct or control transcription of an operatively linked sequence.

Vectors of interest include, but are not limited to, any that are appropriate for conferring expression in any prokaryotic or eukaryotic organism for which a cell that expresses a reporter driven by a gene regulatory region of an organism, cell type, tissue, organ or other selected cell source. Exemplary organisms include animals, such as mammals including humans, bacteria, yeast, parasites, insects and plants.

Vectors for these and other organisms are well known in the art. For example, for mammals, virus vectors include adeno- and adeno- associated virus (U.S. Pat. Nos. 5,700,470, 5,731,172 and 5,604,090), polyoma virus, retrovirus (see, e.g., U.S. Pat. Nos. 5,624,820, 5,693,508 and 5,674,703; and International PCT application No. WO 92/05266 and WO92/14829; lentiviral vectors are described, e.g., in U.S. Pat. No. 6,013,516), papilloma virus (see, e.g., U.S. Pat. No. 5,719,054), herpes simplex virus vectors (see, e.g., U.S. Pat. No. 5,501,979), CMV-based vectors (see, e.g., U.S. Pat. No. 5,561,063), semiliki forest virus, rhabdovirus, parvovirus, picornavirus, reovirus, lentivirus, rotavirus, simian virus 40 and others.

For insects, baculovirus vectors can be used; for yeast, yeast artificial chromosomes or self-replicating 2 μm (e.g., YEp) or centromeric (e.g., YCp) based vectors can be used; for bacteria, pBR322 based plasmids can be used; for plants, CaMV based vectors can be used. See, e.g., Ausubel et al. (1988) In: Current Protocols in Molecular Biology, Vol. 2, Ch. 13, ed., Greene Publish. Assoc. & Wiley Interscience; Grant et al. (1987) In: Methods in Enzymology, 153:516-544, eds. Wu & Grossman, 31987, Acad. Press, N.Y.; Glover, DNA Cloning, Vol. II, Ch. 3, IRL Press, Wash., D.C., 1986; Bitter (1987) In: Methods in Enzymology 152:673-684, eds. Berger & Kimmel, Acad. Press, N.Y.; and, Strathern et al. (1982) The Molecular Biology of the Yeast Saccharomyces, Cold Spring Harbor Press, Vols. I and 11; Rothstein (1986) in: DNA Cloning, A Practical Approach, Vol.11, Ch. 3, ed. D. M. Glover, IRL Press, Wash., D.C.; Goeddel (1990), Gene Expression Technology: Methods in Enzymology 185, Academic Press, San Diego, Calif.; Brisson et al. (1984) Nature 310:511; Odell et al. (1985) Nature 313:810).

Vectors can include a selection marker. As is known in the art, “selection marker” means a gene that allows selection of cells containing the gene. “Positive selection” means that only cells that contain the selection marker will survive upon exposure to the positive selection agent. For example, drug resistance is a common positive selection marker; cells containing a drug resistance gene will survive in culture medium containing the selection drug; whereas those which do not contain the resistance gene will die. Suitable drug resistance genes are neo, which confers resistance to G418, hygr, which confers resistance to hygromycin and puro, which confers resistance to puromycin. Other positive selection marker genes include reporter genes that allow identification by screening of cells. These genes include genes for fluorescent proteins (GFP), the lacZ gene (β-galactosidase), the alkaline phosphatase gene, and chlorampehnicol acetyl transferase. Vectors provided herein can contain negative selection markers.

The reporter constructs are inserted into selected vectors to produce vector constructs. When the vector is a viral vector, the vector constructs are used to generate recombinant viral particles and to transfect, either transiently or stably, suitable eukaryotic, typically mammalian, host cells.

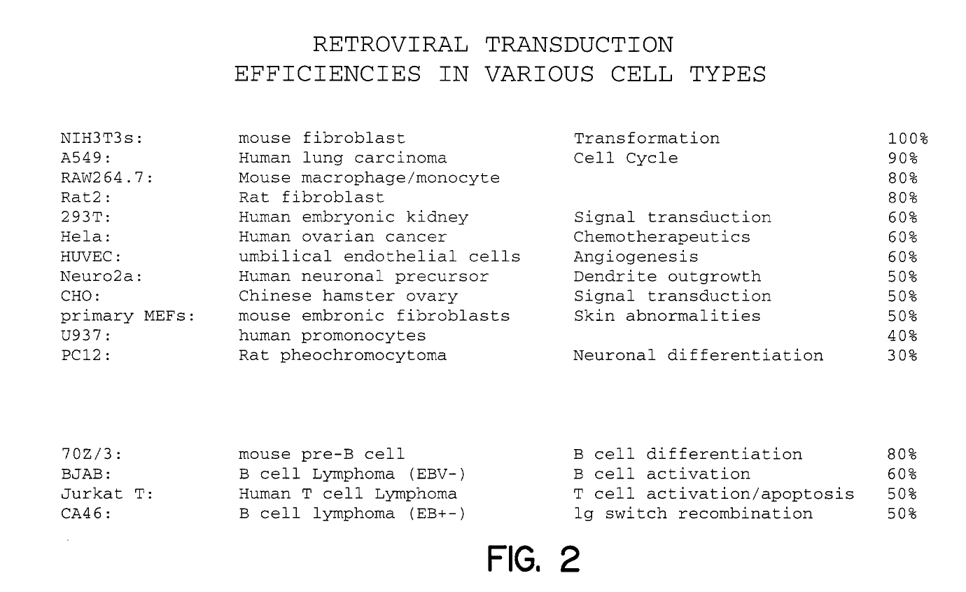

Vectors of particular interest herein are retroviral vectors. Retroviral vectors can be introduced into a large variety of host cells with high transduction efficiencies. FIG. 2 sets forth retroviral transduction efficiencies for exemplary cell types and cellular processes that can be studied using each cell type. A large number of retroviruses have been developed and are well known. Such vectors include, but are not limited to, moloney murine leukemia virus (MoMLV) and derivatives thereof, such as MFG vectors (see, e.g., U.S. Pat. No. 6316255 B1, ATCC acession No. 68754); myeloproliferative sarcoma virus (MPSV), murine embryonic stem cell virus (MESV), murine stem cell virus (MSCV), lentivirus vectors (HIV and FIV vectors), spleen focus forming virus (SFFV); MSCV retroviral vectors, and many others. Retroviral vectors are designed to deliver nucleic acid to a cell and integrate into a chromosome, but are designed so that they lack elements necessary for productive infection.

To generate viruses using the construct described above, retroviral producer cells, either stably derived or transients created by short-term expression of retroviral packaging components, such as structural and functional proteins (i.e., gag-pol and env expression constructs) are plated out for subsequent generation of viral particles encoding the reporter construct. These cells are transfected with the retroviral reporter construct by any suitable method, including direct uptake, calcium phosphate precipitation, lipid-mediated delivery, such as LipofectAMINE (Life Technologies, Burlington, Ont., see U.S. Pat. No. 5,334,761), or any DNA delivery vehicle. Once the DNA enters cells, the cells provide the proteins for production of RNA and packaging of the RNA into the retroviral particles. The virus is released into the supernatant and harvested.

The viral supernatant is applied to a target population of cells, typically the cells from which the inducible promoter was originally identified, and incubated. The cells are treated to permit the viruses to enter the cells (transduce) convert the RNA reporter construct to DNA (via reverse transcription) and integrate into the chromatin of the target cells. Once integrated, if the reporter vector is “SIN”, the promoter regions in the U3 are no longer present and the only promoter remaining is that inserted upstream of the reporter gene.

One exemplary retroviral vector contemplated for use herein is a self-inactivating (SIN) retrovirus. As noted above, self-inactivating retroviruses have the 3′LTR and U3 regions removed so that upon recombination the LTR is gone. A functional U3 region in the 5′ LTR permits expression of a recombinant viral genome in appropriate packaging lines. Upon expression of its genomic RNA and reverse transcription into cDNA, the U3 region of the 5′ LTR of the original provirus is deleted and replaced with defective U3 region of the 3′ LTR. As a result, when a SIN vector integrates, the non-functional 3′ LTR replaces the functional 5′ LTR U3 region, rendering the virus incapable of expressing the full-length genomic transcript.

A viral vector can additionally include a scaffold attachment region (SAR) for circumventing cis-effects of integration on promoter activity; a unidirectional transcription blocker (utb) to avoid competitive transcription; or a selectable or detectable marker. The efficiency afforded by use of these elements (SIN, SAR, utb, selection/detection cassette) for developing reporter gene assays allows rapid analysis of gene regulatory regions.

Thus, also provided are viral expression vectors. In one embodiment, a viral vector with a unidirectional transcriptional blocker and a selectable or detectable marker, or a reporter is provided. In another embodiment, a viral vector can include a scaffold attachment region and a selectable or detectable marker, or a reporter. In yet another embodiment, a viral vector can contain a unidirectional transcriptional blocker, a scaffold attachment region and a selectable or detectable marker, or a reporter. In still another embodiment, a viral vector can include a unidirectional transcriptional blocker, a scaffold attachment region and a selectable or detectable marker, and a reporter. In one aspect, the viral vector is a retroviral vector. In one particular aspect, the retroviral vector has a mutated or deleted LTR so that the vector is self-inactivating.

An exemplary retroviral vector contains the following characteristics: a promoter/enhancer region (LTR, or U3RU5) at the 5′ end; a deleted portion of the 3′ LTR so that the promoter/enhancer function of the LTR is mutated or deleted (SIN, or self-inactivating vector); a psi (ψ) sequence for packaging the vector into a retroviral particle or virion; a region for insertion of a candidate regulatory region (denoted “PROMOTER”), with the upstream promoter sequence being oriented at the 3′ end of this vector, and the downstream portion being oriented at the 5′ end of the vector; a reporter such as a luciferase, including firefly luciferases and Renilla luciferases, beta-galactosidase, fluorescent proteins (FPs), such as (green, red and blue FPs), secreted alkaline phosphatase, chloramphenicol acetyltransferase, lacZ; a scaffold attachment region (SAR) or a sequence that reduces or prevents nearby chromatin or adjacent sequences from influencing this promoter's control of the reporter gene; a constitutive promoter “pro” (such as phosphoglucokinase, actin, or SV40) driving a selectable marker (such as an antibiotic resistance gene, fluorescent, luminescent, calorimetric gene) or gene conferring a selective advantage to cells expressing it; a unidirectional transcriptional blocker (utb) sequence between the marker gene and reporter gene; a “U3” region at the 5′ end not normally found in retroviruses to increase expression, viral titers and thus efficient delivery of the completed reporter gene to cells.

Retroviral expression vector reporter constructs are provided herein that includes one or more of the following characteristics or elements:

1 ) a promoter/enhancer region (LTR or U3RU5) at the 5′ end;

2) a deleted portion of the 3′ LTR, wherein the U3 region, which contains the promoter/enhancer function of the LTR, is mutated or deleted (to produce a SIN, or self-inactivating vector);

3) a psi (ψ) sequence for packaging the RNA genome derived from the vector in cells into a retroviral particle or virion;

4) an inducible promoter of interest (PROMOTER) with, for example, a polylinker inserted in this region for cloning, with the upstream promoter sequence oriented at the 3′ end of this vector, and the downstream portion oriented at the 5′ end of the vector so that in the DNA vector the relation of the promoter to the “reporter” gene is identical to that of the promoter to the actual gene it regulates in the human genome;

5) a selectable marker or reporter, such as, but are not limited to, firefly luciferase, Renilla luciferase, beta-galactosidase, green, blue and/or red fluorescent protein, secreted alkaline phosphatase and combinations thereof, as described above;

6) a scaffold attachment region (SAR) or a sequence or member of a family of sequences (such sequences can be found in the interferon-beta gene (IFN-beta) and are also called insulators; see U.S. Pat. No. 6,194,212) that constrict nearby chromatin, or adjacent sequences from influencing the promoter's control of the reporter gene;

7) a constitutive promoter “pro” (such as, but are not limited to, phosphoglucokinase, actin, and SV40 promoter) controlling expression of a selectable marker or reporter (such as an antibiotic resistance gene, fluorescent, luminescent, calorimetric gene) or gene conferring a selective advantage to cells expressing it, thereby permitting differentiation or isolation of only those cells expressing it;

8) a unidirectional transcriptional blocker (utb) sequence between the marker gene and reporter gene such that marker genes transcribed from the “pro” terminate transcription at some efficiency after the marker to avoid interfering with expression from the “PROMOTER” and the reporter gene transcript RNA, such as via an antisense competition mechanism; and

9) a “U3” region at the 5′ end not normally found in retroviruses, such as a CMV, RSV or other strong constitutive promoter/enhancer sequences to provide for high levels of expression, viral titers and thus efficient delivery of the completed reporter gene to cells.

The structure of the vector can be represented as follows: U3* R U5 ψ pro marker utb reporter PROMOTER SAR ΔU3 R U5, where the order of certain elements, such as the SAR whose effect is position independent, can be changed.

Any retroviral and other sources of these components can be employed. Retroviruses that can serve as sources of these retroviral sequences include, for example moloney murine leukemia virus (MoMLV), myeloproliferative sarcoma virus (MPSV), murine embryonic stem cell virus (MESV), murine stem cell virus (MSCV) and spleen focus forming virus (SFFV). The regulatory region (e.g., promoter) derived from gene chip or by other methods, or gene regulatory sequences are cloned into the PROMOTER region of the vector for generation of responder cells.

The vectors are introduced into cells to produce a collection of reporter cells.

Cells infected with the virus can be selected with agents that eliminate untransduced cells, identify transduced cells, or some method that exploits the “marker” gene to detect transduced cells. In this way, a population of cells expressing the reporter construct is isolated. The marker also can be used to determine the efficiency of viral transduction. Once selected, the cells are treated with the substance or stimulus originally used to identify the inserted regulatory region(S). Studies are performed to recapitulate the magnitude of change experienced by genes under control of the promoter to confirm that the appropriate regulatory region is present in the reporter. If a response that originally observed in the gene expression array screen is not seen at least in part, clones, or individually transduced cells can be isolated and tested to isolate stronger responders.

The thus identified and isolated cells constitute the responder cells for the particular regulatory region and can be used in a variety of ways to manipulate cell function, identify small molecules, genes, and various signals, such as molecular entities, that perturb cell function, particularly those that modulate or effect regulation of the regulatory region, including the promoter.

Parallel Generation of Reporter Cells

As an example of practice of a method for generation of reporter cell, HEK293 cells are plated at 7000 cells/well in 384-well Greiner clear bottom plates using a Titertek Multidrop. Cells incubate for 8 hours before transfection of the reporter libraries. The Hydra-384 (Robbins) with Duraflex syringes is used to mix 2 μl DNA with 8 μl of a premixed solution 61 μl 2M CaCl₂, 440 μl H₂O distributed into a 384-well intermediate plate. Then, 10 ul of a 2×Hepes Buffered Saline solution (HBS, pH 7.0) is mixed with the DNA and pipetted automatically for 5 seconds followed by a 10 μl addition of the transfection solution to HEK293 cells. After transfected plates of cells were incubated at 37° C. for 16 hours, Bright-Glo was added to each well using a 12-head multi-channel pipettor, incubated for 5 minutes then read on the LJL Acquest in luminescence mode. Controls of luciferase expression vectors are used to determine transfection efficiency and CVs.

Recombinase Systems

Recombinase systems provide an alternative way to generate arrays of cellular reporters. Recombinases are used to introduce the reporter gene constructs into chromosomes modified by inclusion of the appropriate sequence(s) for recombination in the cells. Site specific recombinase systems typically contain three elements: two pairs of DNA sequences (the site-specific recombination sequences) and a specific enzyme (the site-specific recombinase). The site-specific recombinase catalyzes a recombination reaction between two site- specific recombination sequences.

A number of different site specific recombinase systems are available and/or known to those of skill in the art, including, but not limited to: the Cre/lox recombination system using CRE recombinase (see, e.g., SEQ ID Nos. 47 and 48) from the Escherichia coli phage P1 (see, e.g., Sauer (1993) Methods in Enzymology 225:890-900; Sauer et al. (1990) The New Biologist 2:441-449), Sauer (1994) Current Opinion in Biotechnology 5:521-527;; Odell et al. (1990) Mol gen Genet. 223:369-378; Lasko et al. (1992) Proc. Natl. Acad. Sci. U.S.A. 89:6232-6236; U.S. Pat. No. 5,658,772), the FLP/FRT system of yeast using the FLP recombinase (see, SEQ ID Nos. 49 and 50) from the 2μl episome of Saccharomyces cerevisiae (Cox (1983) Proc. Natl. Acad. Sci. U.S.A. 80:4223; Falco et al. (1982) Cell 29:573-584; (Golic et al. (1989) Cell 59:499-509; U.S. Pat. No. 5,744,336), the resolvases, including Gin recombinase of phage Mu (Maeser et al. (1991) Mol Gen Genet. 230:170-176; Klippel, A. et al (1993) EMBO J. 12:1047-1057; see, e.g., SEQ ID Nos. 51-54) Cin, Hin, αδ Tn3; the Pin recombinase of E. coli (see, e.g., SEQ ID Nos. 55 and 56) Enomoto et al. (1983) J Bacteriol. 6:663-668), and the R/RS system of the pSR1 plasmid of Zygosaccharomyces rouxii (Araki et al. (1992) J. Mol. Biol. 225:25-37; Matsuzaki et al. (1990) J. Bacteriol. 172: 610-618) and site specific recombinases from Kluyveromyces drosophilarium (Chen et al. (1986) Nucleic Acids Res. 314:4471-4481) and Kluyveromyces waltii (Chen et al. (1992) J. Gen. Microbiol. 138:337-345). Other systems are known to those of skill in the art (Stark et al. Trends Genet. 8:432-439; Utatsu et al. (1987) J. Bacteriol. 169:5537-5545; see, also, U.S. Pat. No. 6,171,861).

Members of the highly related family of site-specific recombinases, the resolvase family, such as γδ, Tn3 resolvase, Hin, Gin, and Cin) are also available. Members of this family of recombinases are typically constrained to intramolecular reactions (e.g., inversions and excisions) and can require host-encoded factors. Mutants have been isolated that relieve some of the requirements for host factors (Maeser et al. (1991) Mol. Gen. Genet. 230:170-176), as well as some of the constraints of intramolecular recombination (see, U.S. Pat. No. 6.171/861).

The bacteriophage P1 Cre/lox and the yeast FLP/FRT systems are particularly useful systems for site specific integration or excision of heterologous nucleic acid into chromosome. In these systems a recombinase (Cre or FLP) interacts specifically with its respective site-specific recombination sequence (lox or FRT, respectively) to invertor excise the intervening sequences. The sequence for each of these two systems is relatively short (34 bp for lox and 47 bp for FRT).

The FLP/FRT recombinase system has been demonstrated to function efficiently in plant cells (U.S. Pat. No. 5,744,386), and, thus, can be used for plants as well as animal cells. In general, short incomplete FRT sites leads to higher accumulation of excision products than the complete full-length FRT sites. The system catalyzes intra- and intermolecular reactions, and, thus, can be used for DNA excision and integration reactions. The recombination reaction is reversible and this reversibility can compromise the efficiency of the reaction in each direction. Altering the structure of the site-specific recombination sequences is one approach to remedying this situation. The site-specific recombination sequence can be mutated in a manner that the product of the recombination reaction is no longer recognized as a substrate for the reverse reaction, thereby stabilizing the integration or excision event.

In the Cre-lox system, discovered in bacteriophage P1, recombination between loxP sites occurs in the presence of the Cre recombinase (see, e.g.,U.S. Pat. No. 5,658,772). This system is used to excise a gene located between two lox sites. Cre is expressed from a vector. Since the lox site is an asymmetrical nucleotide sequence, lox sites on the same DNA molecule can have the same or opposite orientation with respect to each other. Recombination between lox sites in the same orientation results in a deletion of the DNA segment located between the two lox sites and a connection between the resulting ends of the original DNA molecule. The deleted DNA segment forms a circular molecule of DNA. The original DNA molecule and the resulting circular molecule each contain a single lox site. Recombination between lox sites in opposite orientations on the same DNA molecule result in an inversion of the nucleotide sequence of the DNA segment located between the two lox sites. In addition, reciprocal exchange of DNA segments proximate to lox sites located on two different DNA molecules can occur. All of these recombination events are catalyzed by the product of the Cre coding region.

Any site-specific recombinase system known to those of skill in the art is contemplated for use herein. It is contemplated that one or a plurality of sites that direct the recombination by the recombinase are introduced into chromosomes, and then heterologous genes linked to the cognate site are introduced into chromosomes. The E. coli phage lambda integrase system can be used to introduce heterologous nucleic acid into chromosomes (Lorbach et al. (2000) J. Mol. Biol 296:1175-1181). For purposes herein, one or more of the pairs of sites required for recombination are introduced into a chromosome. The enzyme for catalyzing site directed recombination can be introduced with the DNA of interest, or separately.

4. Introduction of the Vectors or Constructions Into Cells to Prepare Collections of Cells

Cell Libraries

The regulatory region-reporter construct can be subsequently transfected into cells either directly such as by calcium phosphate precipitation or using other nucleic acid delivery vehicles, such as cationica lipids. Generally the construct is cloned into a vector or the regulatory region is cloned into a vector upstream a reporter gene in the vector. In some embodiments, the cells into which the reporter gene construct is introduced are the same cells or cell type used in the initial screen or cells of similar origin or lineage. In other embodiments, the cells for example, can be cells that serve as disease models (see, e.g., FIG. 2). Using cells with reporter genes can reconstitute the original response or sets of responses to a perturbation or perturbations.

Subcollections can be prepared by repeating the steps of identifying responder reporter genes and their regulatory regions that respond to selected perturbations. The regulatory regions can be operatively linked to a nucleic acid encoding a selectable marker or reporter and introduced cells to produce sub-collections of responder cells containing gene regulatory region-reporter constructs. Live cellular responder panels for all gene regulatory regions (e.g., promoters), of a particular biological pathway, or a responder cell panel for every gene in the human (or any other) genome therefore can be developed for any cell type or organism. Responder cells can be used for generating an expression profile of any perturbation, such as a test substance or stimulus.

A “live-cellular” responder array of responder cells containing reporters driven by the regulatory regions permits functional studies of the regulatory regions to identify the critical elements that regulate a given gene's expression. Thus, methods of producing collections of cells into which gene regulatory region-reporter constructs have been introduced and compositions containing the cell collections of gene regulatory region-reporter constructs are provided.

A reporter cell array can include a panel of reporter cells. For example, a panel can include plurality of responder cells in an arrayed format. Arrayed format for responder cells include dishes that can accommodate two or more responder cells. For example, microtiter dishes from 6, 8, 16, 24, 96, 384, 1536 and greater numbers of wells for growing different responders can be used to contain a panel (collection) of responder cells.

5. Screening and Profiling the Resulting Collection of Cells

Cells, tissues or organs, or fluids, can be treated with any perturbations, such as a test substance, modulator, condition and stimulus. Examples of test substances include biomolecues, such as known drugs (e.g., chemotherapeutics), drug candidates, small organic compounds (e.g., membrane permeable molecules), metals (cadmium, mercury, lead and others), proteins (e.g., antibodies, receptor ligands), nucleic acid molecules (genes, antisense molecules), cell, tissue, animal, or plant extracts, natural products and toxins such as dioxin. Libraries of tests substances can be used. For example, libraries of biological molecules such as nucleic acid and peptide libraries and small molecule libraries.

Examples of physical and other perturbations that can be used include temperature deviations (high or low) from normal, light/darkness (or altered light/dark cycles), pH, radiation, ultraviolet or infrared light, less than or greater than normal oxygen (e.g., hypoxia), starvation or depletion of one or more nutrients (such as vitamins, lipids and sugars), growth or survival factors (such as serum and perturbationed medium).

Test substances and stimuli can be used in combination with each other simultaneously or sequentially. Thus, a cell can be treated with an ionizing amount of radiation simultaneously with or followed by treatment with a chemotherapeutic drug, for example.

Profiling

Profiling can be accomplished in a variety of ways. For example, solutions containing an input that generates a perturbation of interest (for profiling) is prepared. The solution is transferred to the cellular reporter array with a Hydra (Robbins) or other multi-channel liquid handler and incubated with the array. After a certain time, the cells are treated with lysis buffer and luciferin, the luciferase substrate cocktail and read in a luminometer. The data then can be analyzed to determine which individual cells, and hence regulatory regions, exhibit altered expression.

As discussed herein, a variety of perturbations can be tested and the results cataloged to create databases and also cellular collection with signatures representative of a particular perturbation. The collections can be used to study or identify unknowns (uncharacterized perturbations) and identify cellular pathways and also the targeted promoters or genes of a particular perturbation or input.

### C. Combinations and Kits

Combinations and kits containing the selected regulatory regions, reporter constructs containing the regulatory regions and cells into which the reporter constructs have been introduced, packaged into suitable packaging material are provided. A kit typically includes a label or packaging insert including a description of the components or instructions for use (e.g., growth of responder cells) in vitro, in vivo, or ex vivo, of the components therein. A kit can contain a collection of such components, e.g., a library of promoters, promoter reporter constructs or cells containing promoter reporter constructs representing every promoter for a given cell or tissue type, or organism.

Kits therefore optionally include labels or instructions for using the kit components in a method provided herein. Instructions can include instructions for practicing any of the methods, for example, a kit can include a library of cells each cell containing a distinct regulatory region operatively linked to a reporter in a pack, or dispenser together with instructions for screening and profiling a test substance or stimulus.

The instructions can be on “printed matter,” e.g., on paper of cardboard within the kit, or on a label affixed to the kit or packaging material, or attached to a vial or tube containing a component of the kit. Instructions can additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Kits can additionally include a growth medium, buffering agent, a preservative, or a stabilizing agent. Each component of the kit can be enclosed within an individual container and all of the various containers can be within a single package. Kits can be designed for cold storage. Kits alsp can be designed to contain a panel of responder cells, for example, in an arrayed format on a microtiter dish. The panel of cells in the kit can be maintained under appropriate storage conditions until the cells are ready to be used. For example, a kit containing a plurality of responder cells, in arrayed format, such as in a microtiter plate or dish), for example, can contain appropriate cell storage medium (e.g., 10-20% DMSO in tissue culture growth medium such as DMEM, α-MEM, and other such medium) so that the cells can be revived for growth and studies as described herein.

### D. Computer Systems

Computer systems and programs that include instructions for causing a processor to carry out one or more of the steps of the methods are provided. A computer system or program, for example can manipulate and store data, such as fluorescence intensity of hybridized transcripts, related to gene expression profiling, ranking of genes according to the robustness of their response to a test substance or stimulus, database(s) searches and results for selecting candidate regulatory regions, selection of a candidate regulatory region, primer design for regulatory region cloning. For example, signals of hybridized transcripts can be analyzed and processed by a computer to calculate transcript levels based on hybridization signal intensity. The computer can include hybridization controls in the processing in order to provide greater accuracy in the quantitation of transcript levels. Computer systems and the programs also can include a calculation of the ratio between transcripts whose levels are increased or decreased in response to a test substance or stimulus.

The values representing relative or absolute quantity of transcript levels can be grouped according to whether gene expression is increased or decreased, the fold change in expression (e.g., three-six-fold increase or decrease in one group, six to ten-fold increase or decrease in another group, 10-20 fold increase or decrease in yet another group and greater than 20-fold increase or decrease in the last group and so on). Genes whose expression is increased or decreased also can be grouped according to common functions or participation in a common biological pathway. Thus, the computer systems and programs can further include instructions for grouping genes that share a common response pathway such as a signaling pathway (e.g., TGF-β.

Following quantitation of gene transcript levels, and grouping of genes if desired, the computer can compare the identified gene sequences to one or more sequence databases using sequence comparison software. The computer program, with operator input as appropriate, can select databases searched. For example, following identification of one or more responder genes, the computer can be instructed by the program to automatically query all known sequence databases of all organisms for sequences homologous with responder gene sequences. Any gene sequences identified by such a comparison search can optionally be automatically queried by the computer for the presence of consensus promoter, transcription factor binding protein and enhancer elements, or for sequences having significant homology to such elements. A search of the entire genomic sequence of the identified responder gene, including 5′ and 3′ untranslated regions and introns for such regions can be rapidly undertaken with the computer. When selecting a candidate regulatory region, parameters for the program such as sequence length, the presence of one or more consensus elements, the presence of different genes in the genomic sequence located close to the responder gene, can be preset or be selected by the operator.

Following identification and selection of a candidate regulatory region, the computer can be instructed by a program that also includes instructions for designing a primer to clone the selected region. The program can incorporate instructions for selecting optimal primers for polymerase chain reaction, including any restriction enzyme sites for subsequently cloning the amplified candidate region into a reporter construct. Computer programs useful in designing primers with the required specificity and optimal amplification properties are known in the art (e.g., Oligo pi version 5.0 (National Biosciences).

The data obtained can be manipulated and presented to the user in a convenient format, such as, for example, in a standard relational format or a spread sheet, and also can be stored for future use on a computer readable storage medium, such as a floppy disk, a CD ROM, a DVD or other medium. Specialized tools to visualize the data that are obtained from the present methods in order to interpret the gene expression patterns and the spectrum of biological effects that particular test substances or stimuli have in specific cell types are included. For example, tools can involve multiple hybridization comparisons, or an averaging or summation method that depicts the cumulative results of several hybridization experiments in order to identify genes frequently altered in expression, or tests substances or stimuli that exert the most frequent or greatest effect on gene expression. Many databases, sequence analysis packages, and graphical interfaces are available either commercially or free via the internet. These include the Genetic Data Environment (GDE), ACEdb, and GCG. In many cases, off the shelf solutions to specific problems are available. Alternatively, software packages such as GDE readily permit customization for sequence analysis, data manipulation, data storage, or data presentation.

Computation of hybridization signals, transcript levels, gene expression rankings, gene groupings, database sequence searches, selection of candidate regulatory regions, primer design for cloning candidate regulatory regions and other steps of the methods can be implemented on a stand alone computer system, on a stand alone computer system in conjunction with one or more networked computers or entirely on one or more networked computer systems. A network of computers or communicating over a network (e.g., a local (LAN) or a wide area network (WAN) such as the Internet) allows exchange of hybridization, gene expression ranking, responder gene grouping data, candidate regulatory region selection by database searching, and sharing or distribution of processing tasks among the computers. For example, to select a candidate regulatory region, a local database, i.e., sequences identified through non-public experiments, or global databases can be searched on a local or wide area network. Thus, a computer system can include a plurality of computers, each having hardware components, including memory and processors, sharing data and one or more processor tasks.

An exemplary computer system suitable for implementation of one or more steps of the methods includes a processor element (e.g., an Intel Pentium-based processor) operatively linked with memory. Optional components that can be included in the system include internal and external components linked to the system. Such components include storage medium, such as one or more hard or removable magnetic or optically readable disks. Other external components include user interfaces such as a mouse, keyboard, joystick, monitor and a pointing device.

Typically computers implement one or more steps of the methods following receiving computer readable program instructions. This and other programs (e.g., operating system software) together cause the computer system to function in implementing one or more steps of the methods. Computer programs are typically stored on computer readable medium, such as floppy disks or optical (CD-ROM/RAM) or magnetic disks, or hybrids thereof but can be used by accessing the program over a network. Exemplary operating software (OS) includes Macintosh OS, a Microsoft Windows OS, or a Unix OS, such as Sun Solaris.

Computer readable languages that can be used to write the programs for implementing one or more steps of the methods include C, C++, or JAVA. The methods steps can be programmed in mathematical software packages which allow symbolic entry of equations and high-level specification of processing, including the algorithms used. Such packages include, e.g., Matlab from Mathworks (Natick, Mass.), Mathematica from Wolfram Research (Champaign, III.), and MathCAD from Mathsoft (Cambridge, Mass.). Computer systems and programs that include computer readable instructions for implementing one or more steps of the methods will be apparent to those skilled in the computer programming art.

The sequences of the regulatory regions identified by the methods can be collated into a database, such as a relational database. The databases can contain information representative of regulatory regions from different targets such as different organisms or subsets of genomes or different pathways. For example, information, such as sequences of all regulatory regions of a selected target, such as human, yeast, plant or insect or for a particular pathway, can constitute a database. The databases can include data representative of regulatory regions whose expression is increased or decreased and can link such data to other parameters, such as the source of the region or the perturbation under which expression is altered. For example, all information representative of regulatory regions whose expression is increased under particular perturbations can form database and all regulatory regions whose expression is decreased can be provided as a database. The databases also can be just contain 5′ or 3′ regulatory regions, promoters, transcription factor binding sites and enhancers, if desired.

Accordingly, databases of regulatory regions and/or genes and optionally the perturbation under which the regions are induced or repressed or otherwise altered are provided. Also provided are databases of the profiles or fingerprints obtained by treating panels or collections of responder cells with characterized perturbations.

### E. Automation

The steps of the methods can be automated or partially automated in any combination with manual steps. Operator input, as appropriate, can precede, follow or intervene between the steps, if desired. Software or hardware that includes computer readable instructions for implementing the automated steps also can be included in the systems and programs. An operator can interface with the computer to control automation, the steps automated, and repetition of any step.

For example, the microscope used to detect hybridization of fluorescent nucleic acids hybridized to an oligonucleotide array can be automated with a computer-controlled stage to automatically scan the entire array. Similarly, the microscope can be equipped with a phototransducer (e.g., a photomultiplier, a solid state array, a CCD camera and other imaging devices) attached to an automated data acquisition system to automatically record the fluorescence signal produced by hybridization. Such automated systems are known (see, e.g., U.S. Pat. No. 5,143,854).

The microscope can be operatively connected to a data acquisition system for recording and subsequent processing of the fluorescence intensity information and calculating the absolute or relative amounts of gene expression. Following calculation of relative values, robust responder genes, i.e., those genes whose expression level is increased or decreased by a selected amount as set forth herein are identified and then, if desired, a search of a gene sequence database can automatically follow in order to identify candidate gene regulatory regions. Following identifying candidate gene regulatory regions including the selection of the sequence region, length, and the inclusion of any consensus gene regulatory regions, primers for PCR can be designed. Thus, the entire process or any part of the process from the initial chip scan through designing primers appropriate for cloning a gene regulatory region can be automated.

The following examples are included for illustrative purposes only and are not intended to limit the scope of the invention. The specific methods exemplified can be practiced with other species. The examples are intended to exemplify generic processes.

### EXAMPLE 1

This example shows the identification of inducible regulatory regions by identifying inducibly regulated genes. A method assessing the responsiveness of gene transcript to Hepatocyte Growth Factor (HGF) in a human hepatocyte cell line is exemplified.

Human hepatocyte cells, HepG2 (human hepatoma cells ATCC accession no. HB-8065), were plated at 8×10⁵ cells per ml in a 4 separate wells of a 6-well plate and incubated overnight at 37° C., 5% CO₂. Eighteen hours after plating, 2 wells of cells were treated with 75 ng/ml of HGF continuously for 4 hours, while two samples were left untreated. Cells were harvested by 1×PBS wash, scraped into a 15 ml conical tube and placed on ice. Samples were centrifuged to pellet the cells, flash frozen on dry ice and submitted for RNA extraction.

The following protocol was used to isolate total RNA from the 2 untreated and 2 treated samples:

Isolation of Total RNA from Brain

Tissues were homogenized at maximum speed in 1 ml TRIZOL® reagent (Life Technologies, Gaithersburg, Md.; see U.S. Pat. No. 5,346,994), which is mono-phasic solution of phenol and guanidine isothiocyanate, per 50 mg of tissue using a Polytron (tissue volume should not exceed 10% of the volume of the TRIZOL®) for about 90 secs. The samples are placed in the shaker blocks and shaken at 30 Hz for 10 min. If there is any debris left, the samples are shaken for an additional 4 minutes or so. The samples are then incubated for 5 minutes at room temperature after which 0.2 ml of chloroform per ml of TRIZOL® reagent is added, the resulting mixture is vigorously vortexed for 15 seconds and incubated at room temp for 2-3 minutes, and then centrifuged at no more than 12000×g for 15 min at 2-8° C. The aqueous phase is isolated and 0.5 ml of isopropanol per ml of TRIZOL® reagent is added, incubated at room temperature for 10 minutes, and then centrifuged at 12000×g for 10 min at 2-8° C. RNA is isolated using, for example, QIAGEN'S Rneasy Total RNA isolation kit (available from QIAGEN; see, Su et al. (1997) Bio Techniques 22:1107; Randhawa et al. (1997) J. Virol. 71:9849).

The following protocol was used to generate cDNA then cRNA from the total RNA preparation:

Double-stranded cDNA Synthesis

Variable amounts of RNA can be used, including the following starting amounts:

total RNA-5-10 μg

mRNA-0.5-5 μg.

Determine amount of SuperScript 11 Reverse Transcriptase (RT) enzyme needed:

| | SuperScript II |
|---|---|
| Total RNA (ug) | RT (200 units/ul) |
| 5.0 to 8.0 | 1.0 |
| 8.1-10.0 | 2.0 |

1^{st} strand cDNA synthesis

| reagent | vol. μl |
|---|---|
| RNA | x |
| T7T24 primer | 1 |
| 100 pm/μl | |
| DEPC | y |
| (diethylpyrocarbonate) | |

Incubate 10 minutes at 70° C. → chill on ice

Add the following to RNA mix:

| reagent | vol. μl |
|---|---|
| 5X 1st strand buffer | 4 |
| 0.1 M DTT | 2 |
| 10 mM dntp | 1 |

Incubate 2 minutes at 42° C.

Then add:

| reagent | vol. μl |
|---|---|
| SuperScript II RT | z |
| (200 units/μl) | |

Incubate 1 hour at 42° C.

x+y+z=12 μl in volume

2nd strand cDNA synthesis

| | |
|---|---|
| reagent | vol. μl |

On ice add:

| | |
|---|---|
| DEPC | 91 |
| 5X 2nd strand buffer | 30 |
| 10 mM dntp | 3 |
| E. coli DNA ligase (10 units/μl) | 1 |
| E. coli DNA pol I (10 units/μl) | 4 |
| E. coli RNAse H (2 units/μl) | 1 |

Incubate 2 hours at 16° C. (use microcooler)

Add:

| | |
|---|---|
| T4 DNA polymerase (5 units/μl) | 2 |

5 minutes at 16° C.

Add 10 μl 0.5 M EDTA

Store at 4° C.

Purify ds cDNA

Add to cDNA:

Phenol-chlorophorm-isoamyl alcohol (25:24:1) (162 μl) and then:

Vortex

Pre-spin PLG tube 20 seconds 14,000 rpm

transfer phenol-sample mix to PLG tube

spin 2 minutes 14,000 rpm

transfer top clear layer to fresh tube

add 0.5 volume (81 μl) 7.5 M NH40AC→ mix

add 2.5 volume (608 μl) −20C 100% ethanol (200 proof)

spin 20 minutes 14,000 rpm (15-22° C., not 4° C.)

remove ethanol

add 2.5 volume (608 μl) −20° C. 80% ETOH

spin 5 minutes 14,000 rpm

add 2.5 volume (608 μl) −20° C. 80% ethanol

spin 5 minutes 14,000 rpm

remove ethanol

speed vac →resuspend in DEPC water→ optionally freeze at −20° C. or continue to in vitro transcription reaction

In vitro Transcription

About the half of the ds cDNA reaction is used, if 10 μg of total RNA was used. Usually the fraction of ds-cDNA that corresponds to ˜5 μg total RNA starting material is added. Adding more than this amount to an in vitro transcription reaction can not improve results.

| vol. μl | reagent |
|---|---|
| X | Fraction of ds cDNA corresponding to 5 μg total RNA input |
| Y | DEPC H2O |
| 4 | 10X Hy reaction buffer |
| 4 | 10X Biotin labeled ribonucleotides |
| 4 | 10X DTT |
| 4 | 10X Rnase inhibitor |
| 2 | T7 RNA polymerase |

40 μl total

X+Y=22 μl in volume

Incubate 37° C. for 4-6 hours-gently mixing the reaction every 30 minutes.

The following protocol was used to hybridize the cRNA to gene chips (Affymetrix):

Sample Hybridization

1. Reagents

2. Hybridization mix preparation

3. Chip Pre-treatment and hybridization set-up

4. Non-rotating washing and staining procedure

1. Reagent preparation

| 12X MES stock (100 ml) | 1.22 MES | pH should be 6.5-6.7 |
|---|---|---|
| Reagent | add | without adjustment |
| MES free acid | 7.04 g | |
| monohydrate | | |
| MES Sodium Salt | 19.3 g | |

bring up to 100 ml DEPC water 0.2 μm filter sterilize and store at 4° C.

2× MES Hybridization Buffer (500 ml)

| Reagent | add | Final 2X concentration |
|---|---|---|
| DEPC water | 216 ml | |
| 5 M NaCl | 200 ml | 2 M |
| 12X MES stock | 82 ml | 200 mM |
| 0.2 μm filter sterilize, | 1.0 ml | 0.02% |
| then add: | | |
| 10% Triton X-100 | | |

Store at room temperature for a few weeks or 4C several months

Stringent Wash Buffer (500 ml)

| Reagent | add | Final concentration |
|---|---|---|
| 12X MES stock | 41 ml | 100 mM |
| 5 M NaCl | 10 ml | 100 mM |
| DEPC water | 448.5 | |
| 0.2 μm filter sterilize, | 0.5 ml | 0.02% |
| then add: | | |
| 10% Triton X-100 | | |

Pre-treatment solution (1 CHIP 300 μl-prepared fresh)

| Reagent | add | Final concentration |
|---|---|---|
| 1X MES Hyb buffer | 294 μl | |
| Ac-BSA (50 mg/ml) | 3 μl | 0.5 mg/ml |
| Promega Herring Sperm | 3 μl | 0.1 mg/ml |
| DNA (10 mg/ml) | | |

2. Hybridization Mix Preparation

| | | add | |
|---|---|---|---|
| | 100 μl | 300 μl | |
| Reagent | mix | mix | Final concentration |
| 15 μg fragmented cRNA | A μl | A μl | 0.05 μg/μl |
| DEPC Tx H2O | B μl | B μl | |
| 2X MES Hybridization | 50 μl | 150 μl | 1X |
| Buffer | | | |
| Promega Herring Sperm | 1 μl | 3 μl | 0.1 mg/ml |
| DNA (10 mg/ml) | | | |
| BSA (50 mg/ml) | 1 μl | 3 μl | 0.5 mg/ml |
| 948b 5 nM stock control | 1 μl | 3 μl | 50 pM |
| BioB, BioC, BioD and cre | 1 μl | 3 μl | 1.5 pM, 5 pM, 25 pM, |
| staggered stock (150 pM, | | | 100 pM |
| 500 pM, 2.5 nM, 410 nM) | | | respectively |

A+B=46 μl (for the 100 μl mix) =138 μl (for the 300 μl mix) Store hybridization mix at −20° C.

3. Chip Pre-treatment and Hybridization Set-up

place the chip in the 45° C. oven for 15 minutes

fill the chip with pre-warmed (45° C.) freshly prepared pretreatment solution

place the chip in the 45° C. oven for 15 minutes

place hybridization mix for 5 minutes in the 99° C. heat block

centrifuge hybridization mix for 5 minutes at 14 K rpm

transfer to a new tube without taking the last 5-10 μl (in case you have a little precipitate)

place hybridization mix in the 45° C. heat block for 5 minutes

remove pretreatment solution from 45° C. oven after the 15

minutes incubation

fill the chip with hybridization mix; check for bubbles by turning

the chip upside down

cover septa with tape or tough spots

place chip flat in the 45° C. with glass facing down, or standing

upright in a rack

hybridize for 16-18 hrs

4. Non-rotating Washing and Staining Procedure

The manual procedure includes the following steps:

Fluidics wash—use manualws2 program and 6×SSPE-T with Triton buffer

SAPE stain

AB stain

6×SSPE-T buffer (1 L) (pH should be ˜7.5-7.6 without adjustment)

| Reagent | add | Final concentration |
|---|---|---|
| 20 X SSPE | 300 ml | 6X |
| MQ water | 699 ml | |
| 0.2 μm filter sterilize add to the filtered solution | | |
| 10% Triton X-100 | 1 ml | 0.01% |

SAPE stain (600 μl)

| | | |
|---|---|---|
| 2X MES Hybridization | 300 μl | 1X |
| Buffer | | |
| DEPC Tx H2O | 288 μl | |
| BSA (50 mg/ml) | 6 μl | 0.5 mg/ml |
| SAPE (1 mg/ml) | 6 μl | 10 μg/ml |

AB stain (300 μl)

| | | |
|---|---|---|
| 2X MES Hybridization | 150 μl | 1X |
| Buffer | | |
| DEPO Tx H2O | 146.25 μl | |
| BSA (50 mg/ml) | 3 μl | 0.5 mg/ml |
| Biotinylated antibody | .75 μl | 1.25 μg/ml |
| (500 μg/ml) | | |

Perform the following steps:

remove hybridization mix from chip and save (store at −20° C.);

add 280 82 ul 1× MES Hybridization buffer and perform a fluidics wash

using 6×SSPE-T (10×2);

remove 6×SSPE-T from chip and fill with Stringent wash buffer;

place chip flat or stand in a rack in the 45° C. oven for 30 minutes;

remove Stringent wash buffer and rinse with 200 μl 1× MES hybridization; buffer; remove 1× MES hybridization buffer completely;

fill chip with SAPE stain and place in the 37° C. oven for 15 minutes;

remove SAPE stain and add 200 μl 1× MES hybridization buffer;

perform a fluidics wash;

remove 6×SSPE-T from chip and fill with AB stain

place in the 37° C. oven for 30 minutes;

remove AB stain and add 200 μl 1× MES hybridization buffer;

perform a fluidics wash;

remove 6×SPE-T from chip and fill with SAPE stain;

place in the 37° C. oven for 15 minutes;

remove SAPE stain and add 200 μl 1× MES hybridization buffer;

perform a fluidics wash.

The chip is almost ready to be scanned:

Cover septa with tough spots to prevent chip leaking in scanner.

Ensure the tough spots do not have folds or extend beyond the edge of cartridge.

Check the window for dust or smears —if not clean, use lens paper and water to clean, always wiping from the center out to avoid smearing glue on the glass

If scanning will not be done immediately, remove 6×SSPE-T and fill with 1× MES hybridization buffer. Keep chip stored at 4° C. in the dark; allow the chip to warm to room temperature before scanning. Save the chip after scanning—fill with 1× MES hybridization buffer, store at 4C, dark.

Following the hybridization, the chips are analyzed for relative fluorescence intensity corresponding to each set of oligonucleotides. The location of each oligonucleotide and the gene it represents on the array is known. Using, for example, Microsoft Excel, a list of each oligonucleotide, corresponding gene and relative intensity are recorded and saved. The data sets for treated and untreated are compared side-by-side for average-fold change. The resulting list is parsed by magnitude fold-change and can be represented as text (Excel), or visually (Gene-Spring or Tree-view).

The following details the results of a chip study. Only genes exhibiting greater than 5-fold change are listed. The list begins with the greatest fold induction (FC) and ends with greatest fold repression.

| ProbeSet | FC | AvgD | Avg | AvgDiff | Description |
|---|---|---|---|---|---|
| 40385_at | 19 | 203 | 3851 | 3648 | Cluster Incl U64197: Homo sapiens chemokine |
| | | | | | exodus-1 mRNA, complete cds/ |
| | | | | | cds = (42,329)/gb = U64197/gi = 1778716/ |
| | | | | | ug = Hs.75498/len = 821 |
| 34476_r_at | 15 | 22 | 317 | 295 | Cluster Incl D30783: Homo sapiens mRNA for |
| | | | | | epiregulin, complete cds/cds = (166,675)/ |
| | | | | | gb = D30783/gi = 2381480/ug = Hs.115263/ |
| | | | | | len = 4627 |
| 31888_s_at | 14 | 224 | 3095 | 2871 | Cluster Incl AF001294: Homo sapiens IPL (IPL) |
| | | | | | mRNA, complete cds/cds = (56,514)/ |
| | | | | | gb = AF001294/gi = 2150049/ |
| | | | | | ug = Hs.154036/len = 760 |
| 34898_at | 13 | 342 | 1832 | 1490 | Cluster Incl M30704: Human amphiregulin |
| | | | | | (AR) mRNA, complete cds, clones lambda- |
| | | | | | AR1 and lambda-AR2/cds = (209,967)/ |
| | | | | | gb = M30704/gi = 179039/ug = Hs.1257/ |
| | | | | | len = 1230 |
| 38125_at | 13 | 27 | 3227 | 3200 | Cluster Incl M14083: Human beta-migrating |
| | | | | | plasminogen activator inhibitor I mRNA, 3 end/ |
| | | | | | cds = (0,1151)/gb = M14083/gi = 189566/ |
| | | | | | ug = Hs.82085/len = 2937″ |
| 39105_at | 11 | 21 | 233 | 212 | Cluster Incl Z46389: Homo sapiens encoding |
| | | | | | vasodilator-stimulated phosphoprotein (VASP)/ |
| | | | | | cds = (254,1396)/gb = Z46389/gi = 624963/ |
| | | | | | ug = Hs.93183/len = 2197 |
| 38247_at | 9 | 305 | 966 | 661 | Cluster Incl U67058: Human proteinase |
| | | | | | activated receptor-2 mRNA, 3UTR/ |
| | | | | | cds = UNKNOWN/gb = U67058/ |
| | | | | | gi = 4097702/ug = Hs.168102/len = 1349″ |
| 660_at | 9 | 21 | 193 | 172 | L13286/FEATURE = / |
| | | | | | DEFINITION = HUMDHVH Human |
| | | | | | mitochondrial 1,25-dihydroxyvitamin D3 24- |
| | | | | | hydroxylase mRNA, complete cds |
| 38772_at | 9 | 28 | 271 | 243 | Cluster Incl Y11307: H. sapiens CYR61 mRNA/ |
| | | | | | cds = (223,1368)/gb = Y11307/ |
| | | | | | gi =2791897/ug = Hs.8867/len = 2052 |
| 36345_g_at | 8 | 101 | 853 | 752 | Cluster Incl U34038: Human proteinase- |
| | | | | | activated receptor-2 mRNA, complete cds/ |
| | | | | | cds = (147,1340)/gb = U34038/ |
| | | | | | gi = 1041728/ug = Hs.154299/len = 1451 |
| 1237_at | 8 | 868 | 5313 | 4445 | S81914/FEATURE = /DEFINITION = S81914 |
| | | | | | IEX-1 = radiation-inducible immediate-early |
| | | | | | gene [human, placenta, mRNA Partial, 1223 |
| | | | | | nt] |
| 1379_at | 8 | 331 | 1380 | 1049 | M59371/FEATURE = mRNA/ |
| | | | | | DEFINITION = HUMECK Human protein |
| | | | | | tyrosine kinase mRNA, complete cds |
| 36711_at | 8 | 30 | 323 | 293 | Cluster Incl AL021977: bK447C4.1 (novel |
| | | | | | MAFF (v-maf musculoaponeurotic |
| | | | | | fibrosarcoma (avian) oncogene family, protein |
| | | | | | F) LIKE protein)/cds = (0,494)/ |
| | | | | | gb = AL021977/gi = 4914526/ |
| | | | | | ug = Hs.51305/len = 2128 |
| 35372_r_at | 8 | 55 | 430 | 375 | Cluster Incl M17017: Human beta- |
| | | | | | thromboglobulin-like protein mRNA, complete |
| | | | | | cds/cds = (90,389)/gb = M17017/ |
| | | | | | gi = 179579/ug = Hs.624/len = 1639 |
| 40614_at | 8 | 39 | 298 | 259 | Cluster Incl X75342: H. sapiens SHB mRNA/ |
| | | | | | cds = (310,2100)/gb = X75342/gi = 406737/ |
| | | | | | ug = Hs.173752/len = 2306 |
| 36543_at | 7 | 33 | 170 | 137 | Cluster Incl J02931: Human placental tissue |
| | | | | | factor (two forms) mRNA, complete cds/ |
| | | | | | cds = (111,998)/gb = J02931/gi = 339501/ |
| | | | | | ug = Hs.62192/len = 2141 |
| 37680_at | 7 | 232 | 1640 | 1408 | Cluster Incl U81607: Homo sapiens gravin |
| | | | | | mRNA, complete cds/cds = (191,5536)/ |
| | | | | | gb = U81607/gi = 2218076/ug = Hs.788/ |
| | | | | | len = 6596 |
| 32786_at | 7 | 77 | 536 | 459 | Cluster Incl X51345: Human jun-B mRNA for |
| | | | | | JUN-B protein/cds = (253,1296)/ |
| | | | | | gb = X51345/gi = 34014/ug = Hs.198951/ |
| | | | | | len = 1797 |
| 36344_at | 7 | 131 | 876 | 745 | Cluster Incl U34038: Human proteinase- |
| | | | | | activated receptor-2 mRNA, complete cds/ |
| | | | | | cds = (147,1340)/gb = U34038/ |
| | | | | | gi = 1041728/ug = Hs.154299/len = 1451 |
| 35597_at | 7 | 147 | 966 | 819 | Cluster Incl AJ000480: Homo sapiens mRNA |
| | | | | | for C8FW phosphoprotein/cds = (0,674)/ |
| | | | | | gb = AJ000480/gi = 2274958/ |
| | | | | | ug = Hs.143513/len = 675 |
| 39248_at | 6 | 123 | 772 | 649 | Cluster Incl N74607: za55a01.s1 Homo |
| | | | | | sapiens cDNA, 3 end/clone = IMAGE-296424/ |
| | | | | | clone_end = 3″ /gb = N74607/gi = 1231892/ |
| | | | | | ug = Hs.234642/len = 487″ |
| 36324_at | 6 | 29 | 177 | 148 | Cluster Incl X68487: H. sapiens mRNA for A2b |
| | | | | | adenosine receptor/cds = (332,1330)/ |
| | | | | | gb = X68487/gi = 400453/ug = Hs.45743/ |
| | | | | | len = 1733 |
| 41193_at | 6 | 541 | 2128 | 1587 | Cluster Incl AB013382: Homo sapiens mRNA |
| | | | | | for DUSP6, complete cds/cds = (351,1496)/ |
| | | | | | gb = AB013382/gi = 3869139/ |
| | | | | | ug = Hs.180383/len = 2390 |
| 41524_at | 6 | 96 | 335 | 239 | Cluster Incl L08488: Human inositol |
| | | | | | polyphosphate 1-phosphatase mRNA, |
| | | | | | complete cds/cds = (326,1525)/gb = L08488/ |
| | | | | | gi = 186425/ug = Hs.32309/len = 1705 |
| 277_at | 6 | 984 | 3601 | 2617 | L08246/FEATURE = / |
| | | | | | DEFINITION = HUMMCL1X Human myeloid |
| | | | | | cell differentiation protein (MCL1) mRNA |
| 33146_at | 6 | 634 | 3490 | 2856 | Cluster Incl L08246: Human myeloid cell |
| | | | | | differentiation protein (MCL1) mRNA/ |
| | | | | | cds = UNKNOWN/gb = L08246/gi = 307165/ |
| | | | | | ug = Hs.86386/len = 3934 |
| 529_at | 5 | 55 | 182 | 127 | U15932/FEATURE = / |
| | | | | | DEFINITION = HSU15932 Human dual- |
| | | | | | specificity protein phosphatase mRNA, |
| | | | | | complete cds |
| 2057_g_at | 5 | 52 | 259 | 207 | M34641/FEATURE = / |
| | | | | | DEFINITION = HUMFGF1A Human fibroblast |
| | | | | | growth factor (FGF) receptor-1 mRNA, |
| | | | | | complete cds |
| 36742_at | 5 | 388 | 1252 | 864 | Cluster Incl U34249: Human putative zinc |
| | | | | | finger protein (ZNFB7) mRNA, complete cds/ |
| | | | | | cds = (493,1890)/gb = U34249/ |
| | | | | | gi = 4096653/ug = Hs.59015/len = 2236 |
| 36097_at | 5 | 547 | 2663 | 2116 | Cluster Incl M62831: Human transcription |
| | | | | | factor ETR101 mRNA, complete cds/ |
| | | | | | cds = (100,771)/gb = M62831/gi = 182260/ |
| | | | | | ug = Hs.737/len = 1811 |
| 1890_at | 5 | 1907 | 8242 | 6335 | AB000584/FEATURE = / |
| | | | | | DEFINITION = AB000584 Homo sapiens |
| | | | | | mRNA for TGF-beta superfamily protein, |
| | | | | | complete cds |
| 35454_at | 5 | 32 | 155 | 123 | Cluster Incl AB007919: Homo sapiens mRNA |
| | | | | | for KIAA0450 protein, complete cds/ |
| | | | | | cds = (3226,4503)/gb = AB007919/ |
| | | | | | gi = 3413861/ug = Hs.170156/len = 6946 |
| 2089_s_at | −5 | 117 | 43 | −74 | H06628/FEATURE = /DEFINITION = H06628 |
| | | | | | yl82g03.r1 Soares infant brain 1NIB Homo |
| | | | | | sapiens cDNA clone IMAGE: 44708 5″ similar |
| | | | | | to gb: M34309 ERBB-3 RECEPTOR PROTEIN- |
| | | | | | TYROSINE KINASE PRECURSOR (HUMAN);, |
| | | | | | mRNA sequence |
| 1974_s_at | −5 | 109 | 24 | −85 | X02469/FEATURE = cds/ |
| | | | | | DEFINITION = HSP53 Human mRNA for p53 |
| | | | | | cellular tumor antigen |
| 37487_at | −5 | 114 | 25 | −89 | Cluster Incl AB029016: Homo sapiens mRNA |
| | | | | | for KIAA1093 protein, partial cds/ |
| | | | | | cds = (0,3613)/gb = AB029016/ |
| | | | | | gi = 5689522/ug = Hs.117333/len = 4159 |
| 36048_at | −5 | 107 | 22 | −85 | Cluster Incl AB015342: Homo sapiens |
| | | | | | HRIHFB2436 mRNA, partial cds/cds = (0,674)/ |
| | | | | | gb = AB015342/gi = 3970869/ |
| | | | | | ug = Hs.48433/len = 1065 |
| 32787_at | −8 | 291 | 61 | −230 | Cluster Incl M34309: Human epidermal growth |
| | | | | | factor receptor (HER3) mRNA, complete cds/ |
| | | | | | cds = (198,4226)/gb = M34309/gi = 183990/ |
| | | | | | ug = Hs.199067/len = 4975 |

### EXAMPLE 2

This example describes identification and isolation of inducibly regulated gene promoters. The following methodology was used to identify promoter regions from a sequence database, and is generally applicable to any nucleotide sequence database:

The Unigene system, which is a system for patitioning GenBank sequences into a non-redundant set of gene-oriented clusters, was downloaded from NCBI (see, Schuler (1996) Science 274:540-546). It was parsed for entries where the coding region is explicitly defined (18289 such entries were present in the database). Three hundred bases from the 5′ end of each coding region are assembled into a FASTA™ file. This file was then aligned with the genomic sequence using the BLAST™ algorithm. The target genomic database can be NR or HTGS from NCBI, or the Celera genome assembly. The BLAST alignments were parsed to determine the location of the gene in a larger genomic contig, and up to 10 kB of sequence was taken upstream of the translational start site.

Coding sequences for 12 genes involved in osteogenic/osteoporotic regulation, also represented by probe IDs on Affymetrix GeneChip® arrays, were assembled into a FASTA file, aligned to the Celera genomic assembly and parsed to find the genomic location and sequence of the putative upstream regulatory DNA sequence. The following sequences were identified for CBFA-1 (human core binding factor a subunit-1), MMP-9 (matrix metalloprotease-9), osteoprotogerin, BMP-10 (bone morphogenic protein-10), BMP-7, BMP-2, BMPR1a, FGF6 (fibroblast growth factor-6), leptin, RANK Ligand (RANK for receptor activator of NF-κβ that is a member of the TNF receptor superfamily; RANK ligand is a, Calcitonin Receptor and Parathyroid hormone).

| | |
|---|---|
| CBFA-1 promoter sequence: | |
| TATTGTGATCTAATATGAACCAAAAGCAGATAATGAATAGCACTAGGAA | (SEQ ID No.1) |
| GAACACAGGGATATTTTAGTTCTAACACCCTCCTGTCTCCCTAGCCCTT | |
| ACCTCCCTGCACATTCCAAATAATCTTTTGTAATTCACTGTCTCCGCCC | |
| ACCCCATTTACTTTATGCCACTCCTAGTTACTGTCACACTAGCAAGAAG | |
| TCTAACATGCAGATTTAGAGTGGCATCGATAAATGGCAAAAAAATGCCT | |
| AGAAAATTGGTCTGTTCGCCTTTATAATTTTGGTTGAAAAATACTCCAT | |
| CGCTCCCAACTGATGAAAACAGGAAGCTCTATTCATAAATATAAAATTC | |
| ACTGCCTATGATATATAATCATCCTAATAAGAAAATGAGTTCTATACAT | |
| ACTTGTCCAAAGGGGCAAAAAAGGAGATAGTTTCCCAAAGATGTTTCCA | |
| ATTTTCTTCTGAATCAGAATTAGCAAATCGAGACGACTAACATACTCTG | |
| TCTGTGGGCATTATTCCTTACTACACACAGCATTTTGTAATTTATTTCA | |
| AAGCTTCCATTAGAAACAAAAAAATACATAGCTTCTGTTAACCCACTCT | |
| ATTCTAAGCTCATAGAATCAAATACTGAACAATCTACATTATAACATAA | |
| GCATTTTACTTTATAQAAGATCTGCTATCAGAAACTCTATTAATGTCTA | |
| AACTACTTAAAGAACTATATAAACTCAATACACTTCAATGAAAGACAAA | |
| AAATATTACAATCATAAAGAAAACTAAGTATTCATCCAATAAACTATAT | |
| TACAATCCCTGTCATTCATTTTTTTAAGATCTTCAAACTAGGCATGAGA | |
| TAATGGTATACATGAAACATTACATTTAATCTTTATTGTAAAGGCCGCC | |
| ATCTAATAGATTGATAATAAACTAGACAGACGTGATTTAAAATTTGTAA | |
| AAGAATGCCCAGACTAACACTTTCATGACAGCCAATTATAGTCAAGCCT | |
| AGCAAGCAGTTTGCAACCAGACCTTAAGGTAAACTTTTTTTTTTTTTAC | |
| AATGAGTTACAGATTCACAAGTTTAAGAAGACAAGAAAAAGGAAAACAG | |
| AAGGAATCCAGCCACCCAGCAAATATGAAGCAGACCCCAGAATGTGATA | |
| CAGTCCAAAGATGTGAATTATTGTATATCATCACTGTTGTTCAGAATTT | |
| CACACAGACTCTTGAGCCAATTTTGTTCATTTTTCCACAGACACAATAA | |
| TGAACTAAAAAGAGGAGGCAAAAAGGCAGAGGTTGAGCGGGGAGTAGAA | |
| AGGAAAGCCCTTAACTGCAGAGCTCTGCTCTACAAATGCTTAACCTTAC | |
| AGGAGTTTGGGCTCCTTCAGCATTTGTATTCTATCCAAATCCTCATGAG | |
| TCACAAAAATTAAAAAGCTATATCCTTCTGGATGCCAGGAAAGGCCTTA | |
| CCACAAGCCTTTTGTGAGAGAAAGAGAGAGAGAGAAAGAGCAAGGGGGA | |
| AAAGCCACAGTGGTAGGCAGTCCCACTTTACTTAAGAGTACTGTGAGGT | |
| CACAAACCACATGATTCTGCCTCTCCAGTAATAGTGCTTGCAAAAAAAA | |
| GGAGTTTTAAAGCTTTTGCTTTTTTGGATTGTGTGAATGCTTCATTCGC | |
| CTCACAAACAACCACAGAACCACAAGTGCGGTGCAAACTTTCTCCAGGA | |
| GGACAGCAAGAAQTCTCTGGTTTTTAAATQGTTAATCTCCGCAGGTCAC | |
| TACCAGCCACCGAGACCAACAGAGTCAGTGAGTGCTCTCTAACCACAGT | |
| CTATGCAGTAATAGTAGGTCCTTCAAATATTTGCTCATTCTCTTTTTGT | |
| TTTGTTTCTTTGCTTTTCACATGTTACCAGCTACATAATTTCTTGACAG | |
| AAAAAAATAAATATAAAGTCTATGTACTCCAGGCATACTGTAAAACTAA | |
| AACAAGGTTTGGGTATGGTTTGTATTTTCAGTTTAAGGCTGCAAGCAGT | |
| ATTTACAACAGAGGGTACAAGTTCTATCTGAAAAAAAAAGGAGGGACTATG | |
| MMP9 promoter sequence: | |
| GGCTTATAGAGAACTTATTACGGTGCTTOACACAGTAAATCTCAAAAAA | (SEQ ID No.2) |
| TGCATTATTATTATTATGGTTCAGAGGTAAAGTGACTTGCCCAAGGTCA | |
| CATAGCTGGAAAATGGCAGAGCCGGGATGGAAATCCAGGACTTCGTGAC | |
| TGCAAAGCAGATGTTCATTGGTTAGTGAACTTTAGAACTTCAACTTTTC | |
| TGTAAAGGAAGTTAATTATCTCCATCTCACAGTCTCATTTATTAGATAA | |
| GCATATAAAATGCCTGGCACATAGTAGGCCCTTTAAATACAGCTTATTG | |
| GGCCGGGCGCCATGGCTCATGCCCGTAATCCTAGCACTTTGGGAGGCCA | |
| GGTGGGCAGATCACTTGAGTCAGAAGTTCGAAACCAGCCTGGTCAACGT | |
| AGTGAAACCCCATCTCTACTAAAAATACAAAAAATTTAGCCAGGCGTGG | |
| TGGCGCACGCCTATAATACCAGCTACTCGGGAGGCTGAGGCAGGAGAAT | |
| TGCTTGAACCCGGGAGGCAGATGTTGCAGTGAGCCGAQATCACGCCACT | |
| GCACTCCAGCCTGGGTGACAGAGTGATACTACACCCCCCAAAAATAAAA | |
| TAAAATAAATAAATACAACTTTTTGAGTTGTTAGCAGGTTTTTCCCAAA | |
| TAGGGCTTTGAAGAAGGTGAATATAGACCCTGCCCGATGCCGGCTGGCT | |
| AGGAAGAAAGGAGTGAGGGAGGCTGCTGGTGTGGGAGGCTTGGGAGGGA | |
| GGCTTGGCATAAGTGTGATAATTGGGGCTGGAGATTTGCCTGCATGGAG | |
| CAGGGCTGGAGAACTGAAAGGGCTCCTATAGATTATTTTCCCCCATATC | |
| CTGCCCCAATTTGCAGTTGAAGAATCCTAAGCTGACAAAGGGGAAGGCA | |
| TTTACTCCAGGTTACACTGCAGCTTAGAGCCCAATAACCTGGTTTGGTG | |
| ATTCCAAGTTAGAATCATGGTCTTTTGGCAGGGTCTCGCTCTGTTGCCC | |
| AGGCTGGAGTGCAGTGACATAATCATGGCTCACTGTATCCTTGACCTTC | |
| TTTCTGGQCTCAAGCAATCCTCCCACCTCGGCCTCCCAAAGTGCTAAGA | |
| TTACAGGAATGAGCCACCATACCTGGCCCTGAATCTTGGGTCTTGGCCT | |
| TAGTAATTAAAACCAATCACCACCATCCGTTGCGGACTTACAACCTACA | |
| GTGTTCTAAACATTTTATATGTTTGATCTCATTTAATCCTCACATCAAT | |
| TTAGGGACAAAGAGCCCCCCACCCCCCGTTTTTTTTTTTACAGCTGAGG | |
| AAACACTTCAAAGTGGTAAGACATTTGCCCGAGQTCCTGAAGGAAGAGA | |
| QTAAAGCCATGTCTGCTGTTTTCTAGAGGCTGCTACTGTCCCCTTTACT | |
| GCCCTGAAGATTCAGCCTGCGGAAGACAGGGGGTTGCCCCAGTGGAATT | |
| CCCCAGCCTTGCCTAGCAGAGCCCATTCCTTCCGCCCCCAGATGAAGCA | |
| GGGAGAGGAAQCTGAGTCAAAGAAGGCTGTCAGGGAGGGAAAAAGAGGA | |
| CAGAGCCTGGAGTGTGGGGAGGGGTTTGGGGAGGATATCTGACCTGGGA | |
| GGGGGTGTTGCAAAAGGCCAAGGATGGGCCAGGGGGATCATTAGTTTCA | |
| GAAAGAAGTCTCAGGGAGTCTTCCATCACTTTCCCTTGGCTGACCACTG | |
| GAGGCTTTCAGACCAAGGGATGGGGGATCCCTCCAGCTTCATCCCCCTC | |
| CCTCCCTTTCATACAGTTCCCACAAGCTCTGCAGTTTGCAAAACCCTAC | |
| CCCTCCCCTGAGGGCCTGCGGTTTCCTGCGGGTCTGGGGTCTTGCCTGA | |
| CTTGGCAGTGGAGACTGCGGGCAGTGGAGAGAGGAGGAGGTGGTGTAAG | |
| CCCTTTCTCATGCTGGTGCTGCCACACACACACACACACACACACACAC | |
| ACACACACACACACACACACCCTGACCCCTGAGTCAQCACTTGCCTGTC | |
| AAGGAGGGGTGGGGTCACAGGAGCGCCTCCTTAAAGCCCCCACAACAGC | |
| AGCTGCAGTCAGACACCTCTGCCCTCACCATG | |
| Osteoprotogerin promoter sequence: | |
| AAAATAGGTTAQGCAACTAGTCTGAGGTCACAGAGCTAGGAAAAATTGG | (SEQ ID No.3) |
| AGTTGGGGCTCAAATCTAGGTTACAAAGQCCAGTATCTTAGGTATTCCC | |
| CTAGAATAATCATAACTATAGGAAATATTTCCTATGGGCCAGGCATTGT | |
| GCTGAGTTATTTTACATGCATTACTTTATTTAATGCTCATAATTAGTGA | |
| TTACCATCATTTATATAATTGTTTTTTAAACGCTCCCATTTGCTTTCTC | |
| TTACGTTTCTGCAATATCAGTGTGTTTTTATCTTATAGATGAGGCTCAG | |
| GGAGACGTAAACCTTTCCCAGGQTTAACACTGAAGGACTCAGTTATTGA | |
| TTAGTTTTCTCCAAGGTCTGACACCCACATATTGGCATCATTTTATGTT | |
| CTGAGAAAAACACCTTCAAATAATATCCTAGACAAACATTACTCTAACA | |
| AAAACAATAATACTGCTATTTATATTGTGTTTCACTACTAACACTTGGA | |
| TTGACTTGAGTCCCATGGCAAGTCTAAGTGTTGATATCTCAGGTTGCAG | |
| ATGTCAAAACTACGATTCAAAATACAAGGAGTGATTTGGAGTCATACAA | |
| TTTTGTCCACACTCACTGAGCTACATTTATTCACTAGTTCACTTAAGAA | |
| ACCAGCATGCTGTTACATTCTGGCCCTTGAGQGACAAAGCTGAATGACA | |
| CCCCGTCTTCTGTAATTTGCAGGATGGAACAGTCTGTGGATCCACTTTG | |
| AACTCGTGGTGGAAGGATGTCCCTTGGAAGGGGCAGATGCTCTGATCCT | |
| GGTAAGCCATCCTTGCTCCCCAGGGGTCCCCTCTCCTGATTCTTCACCT | |
| TCCTTCCCTTGAATCTGGTGAAAGGCAGTATTTGCCCTTCTCTGGAGAC | |
| ATATAACTTGAACACTTGGCCCTGATGGGGAAGCAGCTCTGCAGGGACT | |
| TTTTCAGCCATCTGTAAACAATTTCAGTGGCAACCCGCGAACTGTAATC | |
| CATGAATGGGACCACACTTTACAAGTCATCAAGTCTAACTTCTAGACCA | |
| GGGAATTGATGGGGGAGACAGCGAACCCTAGAGCAAAGTGCCAAACTTC | |
| TGTCGATAGCTTGAGGCTAGTGGAAAGACCTCGAGGAGGCTACTCCAGA | |
| AGTTCAGCGCGTAGGAAGCTCCGATACCAATAGCCCTTTGATGATGGTG | |
| GGGTTGGTGAAGGGAACAGTGCTCCGCAAGGTTATCCCTGCCCCAGGCA | |
| GTCCAATTTTCACTCTGCAGATTCTCTCTGGCTCTAACTACCCCAGATA | |
| ACAAGGAGTGAATGCAGAATAGCACGGGCTTTAGGGCCAATCAGACATT | |
| AGTTAGAAAAATTCCTACTACATGGTTTATGTAAACTTGAAGATGAATG | |
| ATTGCGAACTCCCCGAAAAGGGCTCAGACAATGCCATGCATAAAGAGGG | |
| GCCCTGTAATTTGAGGTTTCAGAACCCGAAGTGAAGGGGTCAGGCAGCC | |
| GGGTACGGCGGAAACTCACAGCTTTCGCCCAGCGAGAGGACAAAGGTCT | |
| GGGACACACTCCAACTGCGTCCGGATCTTGGCTGGATCGGACTCTCAGG | |
| GTGGAGGAGACACAAGCACAGCAGCTGCCCAQCGTGTGCCCAGCCCTCC | |
| CACCGCTGGTCCCGGCTGCCAGGAGGCTGGCCGCTGGCGGGAAGGGGCC | |
| GGGAAACCTCAGAGCCCCGCGGAGACAGCAGCCGCCTTGTTCCTCAGCC | |
| CGGTGGCTTTTTTTTCCCCTGCTCTCCCAGGGGCCAGACACCACCGCCC | |
| CACCCCTCACGCCCCACCTCCCTGGGGGATCCTTTCCGCCCCAGCCCTG | |
| AAAGCGTTAATCCTGGAGCTTTCTGCACACCCCCCGACCGCTCCCGCCC | |
| AAGCTTCCTAAAAAAGAAAGGTGCAAAGTTTGGTCCAGGATAGAAAAAT | |
| GACTGATCAAAGGCAGGCGATACTTCCTGTTGCCGGGACGCTATATATA | |
| ACGTGATGAGCGCACGGGCTGCGGAGACGCACCGGAGCGCTCGCCCAGC | |
| CGCCGCCTCCAAGCCCCTGAGGTTTCCGGGGACCACAATG | |
| Leptin promoter sequence: | |
| AGTAAAGTATTTATTCTAGATGQCCATATCCCTACCTAAGACTTGGAGT | (SEQ ID No.4) |
| TTTCTATGACTGGGGAAGAACGGAAGACAAGATATTGGGAAAGACTAGC | |
| AGCCTCTACTAAAAGGGTGATCTGTGTTGATGTGCGTGTGTGTGTGATG | |
| TTTGTATGAGCATGTGTGTTATGTGTTGTGTGTTGGTGGGGCAGATTCT | |
| TGCGAGCACTTTGGTCTCAGATGGACCTGCTACCAGTTCTCTCTGCAGA | |
| CCCCCATAGGTTTCTCCTAAACCTGGCCTCTCCTATTAGGCAGCCTTAC | |
| TCAGCGGCAGCTTCTCAGCTCCATGTTTTCAAGGAACCACAATTTATTT | |
| CCAGCATCCACTGAAGCATATTATCAGTGGTGATAGAGGGGGCTTGTAA | |
| AACTGTTTTTCCACTTAGGTATTAGAGGGTGGCCATTACTTGAGAGTGA | |
| CTATGACCACAGTTAATCTGGTAATAAATTCTCTTGGGTAGGAGGAAAG | |
| GAAAGGATGCTTTAAGGAAGCATCTTGCCGGGAGACACAAAGCTAACAA | |
| GAGTGGAGCCTGCAGCTGGAGCCGCAGAGCCTAATCACTACACCCGCCC | |
| ATCTCTGCTAGGGTTTCATGACTTCGTATCGGGGATTAGCAGTATTTAA | |
| CTCTGTTGCACAAACATTTGGTGTATTATTCAGGTAACAAGTAGCTAAT | |
| AGAGGAAGTTTTACTTTTTTAAGACATAAATTTGCCTTTTCCCAAATTA | |
| CTTGGTACATAGTACTTTTCATGTTTGAAGTTGAGATGTGGGTACAATA | |
| CCATAGCTTTATTCCAGAGCAGGGTATTTGTTTCCAAATGCCATGTTCC | |
| CAGCAGCTGCCCTTGACTGGGAATTGGGGTGTGATTTGGGCTTTTCCTT | |
| AAATCCTTGAGGAGCTGGAGGGGTGGGTGGCTCGCACTCCTGCTTTCTG | |
| GATCTGAATCCTGACTCTGTCATGGACCTGTTTGACTTTGGGCAAGTTG | |
| ACTCCTATTCCTGAGCCCCATATTTTTCTCTTCTGTAAAATTCAGATTA | |
| AAAAAACATGGCTTTGATCAAACATTATAAATAATATATAGACAGACTG | |
| CTTGTTTTTATTGTATTGCCAGAAATGAATCCTACTAATATTGCCATCT | |
| ATGGACAGAAAATGTATTACCTGTCTTCATCAAGACCCAGACGAGGAAG | |
| AACACGAAAAGCGGAGATTAATTTTACTGCCATCTCCAGAACCGTCATC | |
| CTAATATTTACTTACATTTTATTATTATTTCAGGCTCATGCACATATAC | |
| TTAGCATGGATCATTGGCCACAGACTCGCATACATTTAACTTTATTACC | |
| TTTTGCCTCATGTATCTCATTAAAATTTTGCTGCTTAATCAAGGATCTG | |
| CATATTATTTTAATTTTAGAATTCACAGTTCCAAGACTTTGAAAGTTTC | |
| AAGCGTTCTGGGTGAATGTGTTATGCTCTCTCCCGCCACCATGTCTTTA | |
| TACCCCCTGATTTCTCAGCCACTATGGCAACCACTTTCTACTCTTAGTA | |
| GCCCATATTTAGTCCAATCCCCAGCTCAGGAGACACTTCTTCCAGGGAG | |
| CCCCCTGTGCCTTCCAGTAGTATCTTGTACCTGCCCTTTTTGCAAAGCT | |
| CTTTCCTCCTGGCTTAGAATGGCCCATTGACCTGTTTGTTTCTCCTATT | |
| AAACTGTAAGCCACTCGAGGGTAGAGAGCATCTGTTGTTCACCATTGCA | |
| TCCTCGGTGCTGAGCACTGCGTCTGACATATTATTTAGAAGGTCAGTAA | |
| GTGCTAGTGGGATTCAGGCTCCCAGTGGGTGGGAGAGAAAGGACGTAAG | |
| GAAGCAAGTGGTAAAGGCCCTCACAGAGTATCAGCAGGCTGGTGTGAGG | |
| GAGAAATGCAGAGGATGGGTQAGTAGCATAATCGCTAATGATAGGGTAA | |
| TGATAGAGCACATTTCACAACACCTTTAAGCCCTTTCACGTGCATCAGA | |
| TAATTTGATCCTCATAAAAGCCTAGAGATAGATATATTACAGGGATGAA | |
| GGTGGAGTATTTTGTGGTTATGTGATATGTTTAAAATTATGCAGTGAGT | |
| AAATGACTGGGTTCAAACCAGACCTTAAAAGTCTGTTATCTTTCCCTCG | |
| AGCATGCAATGAAGTCTACATCATCCCTACCATGTCCATTTGATCACAC | |
| CCTGGCCTCACAGCTCTGTGGTCTACAGGATACCTCATGGTGGTTTTAT | |
| TGACCAGACAATAATCCTCTTTCTAAGGGGATGCATTTCATTAATACAT | |
| ATGTAGATCATGAATTGTCTTTGACTTTGAGGGGATGGTAGCCAGAGCA | |
| GAAAGCAAAGCTGATTTTCATCCCCGTCTGGTAATGTGGTTGGTAATGT | |
| GAAGATGGGTGTATTCTGAGATACCGGCTCCTTGCAGTGTGTGGTTCCT | |
| TCTGTTTTCAGGCCCAAGAAGCCCATCCTGGGAAAATG | |
| FGF6 promoter sequence: | |
| CCGTGGTGACAGTAGGAACAAGTGGTGCCTATGTCCCTCCCCATTCAGT | (SEQ ID No.5) |
| TTACCAGCTGAGGGTAAAGACAGACATCTGGGCTTCACAGGATTTCAGA | |
| AGGCATGTCTAGGGCAACACTAAACACATGGCTTGACAGAAATTTGAAC | |
| CAAAGCATCGAACCCAGTGAACGAGGCAGAAGGGCAGAGAGAAGGCAGG | |
| TAGAAGCCACAGACCAGAGGCTGGGACCCAGCGCACAGCAGAAGGTTTA | |
| GAATCAGAGGGAAGGCGGTGGTGCCTCAGTAQAGTCCTTGGGCCATGGA | |
| ACTCACCCCAGGAGCTTTTCCAGGCTGCCTGCAGCCTGCAATGTGGGTG | |
| TAGAGTGTGGCTAAGGGAGCTGCCTGCTGGGACCAGCTCTACTGCTCAG | |
| GACACTCAAATCCATCTGTATGCCACTGTCATCACCCCACACATACTCT | |
| CTCCAATCCCGGCAAAATCAGTGCTAATGTCTCACCAACAGATTAAGGC | |
| CTGGATTGAAGTACAAGAAACAGGATTTTTAACTCAAGTTAATTCAATT | |
| CCCCAGCGACCCTTGTTAACTTATTCACCCTCAGAGACGTATTAATAGT | |
| TCTGTCTTATATTGTATAQAAATTTGTGCAGTGAGTTTTCTGGTAGCTT | |
| TACATTTTTTTTCTCACTTCAGTTAGACATGTAATCTATTTAAAAGTAA | |
| TATGGGAATAAGATAAATCAGTGTAGGAATAACTTCCTGGCAGAAATAT | |
| TTTTACTAGTTTCTGAGTGTAATATCAGCCCAGCAAAAGTTATCTGCAA | |
| ATATAGAAGTTCTCATGTACATCAAAGACACTCAAGTTTTTTTTAAGAA | |
| ATAAATCATTTTATGCTACTGAAATAACTCTGTGATGTGCTATTGGCAT | |
| TTAAGGAGCTAAACAGACTCTATGGQCCAGCCAACTTCTACTGCAAGCA | |
| TTAGACATGCACAGGCTTTAGACTCAGGCACACCTTAGAAGTTCTGGCT | |
| TTGCTACTTATTAGCTATGGTAACTCGGGCAGGTCATTTATCCTCTCTA | |
| AGCCTCAACTTCCTCATCTGTGAAATGGGAATAATATCAGTCACATGCC | |
| AGGGATAAATCCAGGGAGAATQGCCAGGGGGCTGTGTCAAAGGCCAGAC | |
| ACAACTTCCACCCCAGGTGAATGTTGGGACCAGGACAGTGAGCAGGCAA | |
| ACCTTGCCCTTGCCCTCCTTCCCTCCACAATCTTAAAGCTCCTTGAACA | |
| ACCCCCATCCCCACCCCCTGAGAATGTCTGTGCCCTCCTGCTGAAAGGG | |
| TTTGGCCTTTCAGTGTTCCCCTCCACCATGAGCTGTTTCCATGAAAAGA | |
| TCTCAAGGGTGACTTGAGGCTACGGTCATCACTACCACAAGCCTTTTCC | |
| CATCCCTGCCTCTACCTATTGCCCTCTAAATAAGGAAGCCAGCGCTGCC | |
| AGGCAAAGAACTTCTGCCCAATATGGGTCCTGGGTGGCCTCTCGCCTCT | |
| CTCTTTCCCTGGGCCCCCAGCCAGCTCCCCCCTCCCCCAGAGATGCTCC | |
| CTGCTCACTTCATTCCTGCCTCATAGTTGGAATGACAGTGGCTCCCAGA | |
| ACCCCTGGGGAGTGTGGAGGQTGATGGGGGTCTGGGGAGGCAGCCAGGC | |
| CCAAGAGCAGGTTAATGTTACAGCCCTGGATAAGTGAGCTGGGCGGGTT | |
| GACGTCAGGGCGATGATGGGTGGAGGGGAGGGCCGGGCTGCTGAAGCAA | |
| CTATAAAGATAGGTCAAATCAAATATCATCAACTAGGGACGGAGCAAGC | |
| GGGCGAGCTAGAGAGCGTCCCCGAGCCATGGTCTCTACCGGCCGCGGCT | |
| CAGCCTGGGTCCCTCTGCTCTCAACCCGAGTGCCCGATGGAGGCTTTGG | |
| TTTCATGTCAGCAGCCTTCATCTGCCTTCCAAAAATAAGCCCCTGCCGC | |
| CATGCCGGAGGGAGAAAAACAAGAAGGGCGGTATTTTTAGGGCCATTAA | |
| TTCTGACCACGTGCCTGAGAGGCAAGGTGGATGGCCCTGGGACAGAAAC | |
| TGTTCATCACTATG | |
| BMP7 promoter sequence: | |
| CTGCCCAGCATGGTGCTTGGCCCTGGGACTGGCCACATAATATCTGGGC | (SEG ID No.6) |
| CAGGTGCAAAATTAGTACGGGGCAGGGGGTACTTTGTTCATAGGTGATT | |
| CAGAACCACATATGGTGACCTCAGAGTAGGAAACCAAGTGTGGGGCCCT | |
| TAAGAGCTGGGGGGCCCTGTACGACTGTCCAGGTTGCAGGCCCCACAGC | |
| TCGCCTCCTGATATCCTGTGCTCCATGCTTGTCTGTTGAAGGAAGGAGT | |
| GAATGGATGAAGAGCAGGTGGTGGGGGTGGTTTGAGGGCCTTGCCTGGT | |
| GGGTGGGTAGAGGCCCCTCCCTGGCATGGGGCTCAAGACCTGTTCCATC | |
| CCACAGCCTGGGGCCTGTGTGTAAATGGCCAGGACCTGCAGGCTGGCAT | |
| TTTTCTGCTCCTTGCCTGGCCTCTGGCCTCCCCTTTCTCCACCCATGTG | |
| GCCCCTCAGGCTGCCATCTAGTCCAAAAGTCCCCAAGGGAGACCCAGAG | |
| GGCCACTTGGCCAAACTACTTCTGCTCCAGAAAACTGTAGAAGACCATA | |
| ATTCTCTTCCCCAGCTCTCCTGCTCCAGGAAGGACAGCCCCAAAGTGAG | |
| GCTTAGCCAGAGCCCCTCCCAGACAAGCGCCCCCGCTTCCCCAACCTCA | |
| GCCCTTCCCAGTTCATCCCAAAGGCCCTCTGGGGACCCACTCTCTCACC | |
| CAGCCCCAGGAGGGGAAGGAGACAGGATGAACTTTTACCCCGCTGCCCT | |
| CACTGCCACTCTGGGTGCAGTAATTCCCTTGAGATCCCACACCGGCAGA | |
| GGGACCGGTGGGTTCTGAGTGGTCTGGGGACTCCCTGTGACAGCGTGCA | |
| TGGCTCGGTATTGATTGAGGGATGAATGGATGAGGAGAGACAGGAGAGG | |
| AGGCCGATGGGGAGGTCTCAGGCACAGACCCTTGGAGGGGAAGAGGATG | |
| TGAAGACCAGCGGCTGGCTCCCCAGGCACTGCCACGAGGAGGGCTGATG | |
| GGAAGCCCTAGTGGTGGGGCTGGGGTGTCTGGTCTCAGGCTGAGGGGTG | |
| GCTGGAAAGATACAGGGCCCCGAAGAGGAGGAGGTGGGAAGAACCCCCC | |
| CAGCTCACACGCAGTTCACTTATTCACTCAACAAATCGTGACTGCGCAG | |
| CTACAGTGGCTACCAGGCGCTGGGTTCAAGGCACTGCGGGTACCAGAGG | |
| TGCGGAGAAGATCGCTGATCCGGGCCCCAGTGCTCTGGGTGTCTAGCGG | |
| GGGTAAGAAGGCAATAAAGAAGGCACGGAGTAACTCAAACAGCAATTCC | |
| AGACAGCAAGAGAAACTACAGGAAAGAAAACAAACGTGCGAGGGGCGAG | |
| GCGAGGAAACAACCTCAGCTTGGCAGGTCTTGGAGGTCTCTGGGAGGAG | |
| AAAGCAGCGTCTGATGGGGGCGGGAGGTGGTGAGTGGGGAGAGGTCCAG | |
| GCGGAGGGAATGGCGAGCGCAGAGACAGGCTGGCAACGGCTTCAGCGAG | |
| GCGCGGAGGGGTCAGCGTGGCTGGCTTAAAAGGATACAGGGACTGAGGG | |
| GCAAGACCGGCTCAAGGGTCACCGCTTCCAGGAAGCCTTCTATTTCCGC | |
| GCCACCTCCGCGCTCCCCCAACTTTTCCCACCGCGGTCCGCAGCCCACC | |
| CGTCCTGCTCGGGCCGCCTTCCTGGTCCGGACCGCGAGTGCCGAGAGGG | |
| CAGGGCCGGCTCCGATTCCTCCAGCCGCATCCCCGCGACGTCCCGCCAG | |
| GCTCTAGGCACCCCGTGGGCACTCAGTAAACATTTGTCGAGCGCTCTAG | |
| AGGGAATGAATGAACCCACTGGGCACAGCTGGGGGGAGGGCGGGGCCGA | |
| GGGCAGGTGGGAGGCCGCCGGCGCGGGAGGGGCCCCTCGAAGCCCGTCC | |
| TCCTCCTCCTCCTCCTCCGCCCAGGCCCCAGCGCGTACCACTCTGGCGC | |
| TCCCGAGGCGGCCTCTTGTGCGATCCAGGGCGCACAAGGCTGGGAGAGC | |
| GCCCCGGGGCCCCTGCTAACCGCGCCGGAGGTTGGAAGAGGGTGGGTTG | |
| CCGCCGCCCGAGGGCGAGAGCGCCAGAGGAGCGGGAAGAAGGAGCGCTC | |
| GCCCGCCCGCCTGCCTCCTCGCTGCCTCCCCGGCGTTGGCTCTCTGGAC | |
| TCCTAGGCTTGCTGGCTGCTCCTCCCACCCGCGCCCGCCTCCTCACTCG | |
| CCTTTTCGTTCGCCGGGGCTGCTTTCCAAGCCCTGCGGTGCGCCCGGGC | |
| GAGTGCGGGGCGAGGGGCCCGGGGCCAGCACCGAGCAGGGGGCGGGGGT | |
| CCGGGCAGAGCGCGGCCGGCCGGGGAGGGGCCATGTCTGGCGCGGGCGC | |
| AGCGGGGCCCGTCTGCAGCAAGTGACCGAGCGGCGCGGACGGCCGCCTG | |
| CCCCCTCTGCCACCTGGGGCGGTGCGGGCCCGGAGCCCGGAGCCCGGGT | |
| AGCGCGTAGAGCCGGCGCGATG | |
| BMP10 promoter sequence: | |
| GTTGACATCTGTGTGTGTGTGAAGATAAATGGGTGCCTGTTTGGATGCAG | (SEQ ID No.7) |
| GACATGATACAGGGCATTGCTGGTATGCTGTCAGAAACCTCATGTGAAAA | |
| CGAACCACCCGAAGGACGGCTTCTGGCCCTTGGAGTCACTCACTCACTTG | |
| TGGGACTGTTCAGGGTATAATCTGTCTCCAGTCTACAATTGTCGTTTTAC | |
| TATGGGAATAGAAAGTTTGAATCAAAATTGAACATTGAATCAAAATCAAA | |
| ACTATTAAACAAATAGACAATTAACAACTACTAAACAAAATATGGTTCTT | |
| TCTATGGTAATTTAAAAAATGGCTGTAACATTGTACATTTTAGGAGGAAA | |
| AAGAATCAAAAGATGACTAGAAACCTAAGTGAGCCTGGAGAAAAAGTTAA | |
| GTGGAGACATTGTAGCTAAACGATGAGCATGAATATAGGAAAATTTAACC | |
| TAGAAACTGAGAAAGGATTCCAGTGAACCAAATATCTTGACACAGCCCTT | |
| GGAACACAGCACCAGGACGCGTGAGTAATGGTGTGCACGTCAGAAAGATA | |
| CCAGAACTACCACCTCAGTGGGAAAAACATCCCCTGGGCTTGTCCGCAGG | |
| GCCTCTCTGGCTGCACCCCGGCTGCTACTGTCACTAGTTAGAATGGAAAA | |
| TGTGATGAACCTGATTTGTCTTTCCTAATCTGGACACACAATCGATTCTA | |
| CCATTTTTATTTTCAGGACCAAGGCATTTGGCGTTTTTTGTGTGCCTAGT | |
| AATGTTGTTTGCCGAGTGTATTAGTCAGGGTTCTCTAGAGGGACAGAACT | |
| AATAGGGGATGGAGATATATTTCTGAGTTTATTAAGTATTAACTCACACG | |
| ATCACAAGGTCCCACAATAGGCTGTCTGCAAGCTAAGGATCGAGGAGAGC | |
| CAGTCCAAGTTCCCCGACTGAAGAACTTGGAGTCCCATATTCAAGGACAG | |
| GAAGCATCCAGCATGGGAGAAAGATAGGCTGAAAGTCTAGGCCAGTCTCG | |
| TCTTTTCACGTTTTTCTGCCTGCTTTATATTCTAACCGTGCTGGCGGCTG | |
| ATTAGATGGTGCCTAGCTAGATTAAGGGTGGGTCTACCTTTCCCAGCCCA | |
| CTGATTCAAATGTTAATCTCCTTTGGCAACACCCTCACAGACACACCCGG | |
| GATCAATACTTTGCATCCTGCAATCCAATCAAGTTGACAGTAAGTATTAA | |
| CCATCACACCAAGCTTTTGCTGGAGCCTCTTGATGACAATTTTGATTGAG | |
| TCAGAAGGATGAATTTCGCAGAGATGTTGGTTATATTAACAACTCATTGC | |
| ACAGATGGAGGACCTGAGGTCCACATCCAGCTACAAATTTCTGCCTGCCT | |
| CCTGCCTCCAGGCTGATCTGGGGACGTGGTGGCCTCTCAGCATTATTGCC | |
| CATGCCCTAGTCTGGTAGAAGAGTGGTTTAAAAGTGTGACTGTTTTATTC | |
| TTCATAAGAATCAGGCTGCCTTGGTTGAAATTGTGGCCCCATCACTTTGC | |
| AACTTTGTGGCCTCTGGCAAGCTATGGCACTTCACTGACCCATATATGTG | |
| ATGGAGATAATGATACGGTTATTACAGGAGCACACTTGATGATAGGTGTA | |
| AAGCACTCAGTACAATGCCTGTTTGTAGGAAGCATCTAATAAATTCTAGT | |
| TGCCAGTATAACTAAGCACTTGCCCTATTTTTCAAATGCTATTTTAGCCA | |
| GATCAAATAGGTAGGAAAAAGCCTGTCAATCATGAAGTTTATACTTTCCT | |
| GTTTCTAAAAAGGTACACTTCTAAAAATTTATATAATTCATTTATAGCTA | |
| TTAACTTAAACTTGGAAAGTTTGGATATTTGGTCTGTCTTCACAAGTGTT | |
| TATCTGAGCCCTACCTCTCAAATTAACATGTATCACCATTGATGTGCATT | |
| ATGTTGATTCTTATACCTATTATATGCATGTGTGAAACTAAGCCCCATAA | |
| AAACAGAATTTAGGCATTCCTGCTGAAAGGAAGTGAATTGAAGGGAAGAG | |
| AAGCAGAGCCTTTGCAAAGAGAAAATTGTCCTATCTCTCAACCAGTGTCA | |
| GAATGTGGAAATGTTTACAAAATGCTCATTAAAAGAAATAGGGATTGCAA | |
| GATAGAAACAAATTCTGGTGCACAAGTTTACACTAGGGAGAAAGAAAGGC | |
| TAGGCCCCTATAGGGGATTTTGTTATCCAATTACTGCAACCTGACTTTTA | |
| GGGGGAGAGGAAGAGTGGTAGGGGGAGGGAGAGAGAGAGGAAGAGTTTCC | |
| AAACTTGTCTCCAGTGACAGGAGACATTTACGTTCCACAAGATAAAACTG | |
| CCACTTAGAGCCCAGGGAAGCTAAACCTTCCTGGCTTGGCCTAGGAGCTC | |
| GAGCGGAGTCAGT | |
| BMPR1A promoter sequence: | |
| AATCCATCTATTTTACTCTTTATAAGAAATCTTTTAAATGAAAATAAAGAT | (SEQ ID No.8) |
| AGGTTGAAAGTTAAACAAAATCAGAAAAAACATACCATACAGAGTAAGCAT | |
| ATGAAAACTGCTGTGGCAATGTTAATAAAAAATAAAGTAGACTTTAGGACA | |
| AAAAGTGATATCTGAGATTAAGTGGAGATCTTCACAGTTATCAAAATATTA | |
| ATTTATAAGATATAAAAATCTAAAGATTCAAAATATTCTAAATATGTATGT | |
| GCCTCATAACAGTGCTTCAAAGAACAGGAAGAAATACTGAAAAAAATGAAA | |
| GAAAGGTAGGAATCCATAATCGCAGATTGGAAAAATCCACATTTATTTGTT | |
| TGCCAAGAGAGACCATGCACTGAGCCATAAGTTAAATTTCAATAAACTTCT | |
| AAAGTTTGACATCTTAGAGAGTATGTTCTCAGATCATAAACATCCAGTGTA | |
| GAAATCAAAAATATAATATTTAATAAAGCTCAAATATTTGGAAATTAACAA | |
| AAAATAAATCACAAGAGAAATTAGAAATTATGTTAAATAAATGACAATGAA | |
| CATAAAGCATTCCTGAATTCATGAGAAACAGCTAAAGAACTGCTAGAAGGA | |
| AATCTATATTTAAAAGTTTATATGATAAAAGAAGAAAGGTGTAAAATCATA | |
| ATTTAACTTTCCAAATTGATAGGTAGAAAAAGAAAATGAAATTTAAAACCA | |
| AAACAGGTCAAATGAATAATATAATAAATAGAACAGAATCAATAAAAACAC | |
| AAAAAATAAAAAGGCAGAAGTTTTTTTGGAAAAGATTAGGAAAATTGATAA | |
| ACCCCTAACATAAGTGATCAATAAAAGGAGAAAAGCACAACTTAATCATTTT | |
| AAAAATTACACAGGGGATATCTATATAGATGCTATAGACTTCAAGAAGATAA | |
| TAAGGCAATTTTTAAAACTGCCAATTGCCAATGATTTGACAATTTAGATGAA | |
| TTGAACAAATTACTTGAAAAATACAATATATCAAAAATTGACCCTCCCTAAA | |
| GATATTAATACAAAACCTATCTAACCCTATGTCTAATAAAAAATAGCCAATA | |
| CAATGCACGAAGAAAACTAGAGACTCAGATAGTTTCACTAGGAAATTTTATC | |
| AAGCATTTTAAAGAGAATTAATTTTAATCTGAAGTTACTTTAGAAAACAGAA | |
| GAGGAAGTGCATTTCCCCGATCATTTGTTGATGCCAGTATACCCCAATAAAA | |
| AACCTGACAAAAACATTATAAGAAAATAAAATTATAGACCAATATATTTTAT | |
| GAGAGGATGTCAAAATTCTTAACCAAACATTAGTCAATTGAATCATCCAATA | |
| TATAAAAATGATAATATATCATAACCAAATGGAGATTAATTCACAAATGCAA | |
| AGCTGCCTTCATATTTTAAAATTCAATTTGCATAAATTGTCCCCGTTAACAG | |
| AATAAAGGAGAAAATCCTTATGTTCATTTCAGTAGGTTTCGAAAAGCATATG | |
| ACAAAATGCAAAACCATTTTGTTATAAAAACTCTCTGCAACTTAGGAATAGT | |
| AGGGGACCTACTGAATCTGATAAAGGGTGTCCATAAAAAAATATGCAGTTCA | |
| CATCATACTCCATAGTGAAATATTAGGTTTCCCTTTAAAATTCAGAACAAAG | |
| TGAAGATGTCAGCTCTCGCCATTTTTAGTTAACCTTGGCATAAAGATTGCAA | |
| AGGAAGAAGTAAGCCTGAATGTACTTGCAGGTAAAATGATTGTTTATGTGTA | |
| CGTTTCTAAAGCATGTAGTTTAAAACTACTAGAATTAATAAAGAAATTAAGC | |
| ATGGTGGGTGCTCCCGAATCGATGAGGAAAGCCGCTCTCCCCGGCAGATCCT | |
| CCCGGCCGGGGCGCCTCCATCACCCTGCCTGCGCCTCGGCACGCTGGCAAGG | |
| AGCCCGGGAAGAGACGCCGGGAGCGACTTATGAAAATATGCATCAGTTTAAT | |
| ACTGTCTTGGAATTCATGAGATGGAAGCATAGGTCAAAGCTGTTTGGAGAAA | |
| ATCGGAAGTACAGTTTTATCTAGCCACATCTTGGAGGAGTCGTAAGAAAGCA | |
| GTGGGAGTTGAAGTCATTGTCAAGTGCTTGCGATCTTTTACAAGAAAATCTC | |
| ACTGAATGACAGTCATTTAAATTGGTGAAGTAGCAAGACCAATTACTAAAGG | |
| TGACAGTACACAGGAAACATTACAATTGAACAAGT | |
| Rank ligand (Tumor necrosis factor (ligand) super- | |
| family, member 11) promoter sequence: | |
| GTATTTACCATGCACCTACTATAGCAGGCAACATTTTTAGGAAATGGTGAAT | (SEQ ID No.9) |
| GTTACAGAGGTGAATAATACAGCAAGAGTCGTTGAACATATGGAGTTTATCT | |
| ATTAGTTGGGGAGTGAATGTTGACAAAGGAATAAGTAAATACATAGGCAAGA | |
| AAGATACATTACCTGTGAAACAGCAGCAGGTAGACTGACAGTGGAGTATCTA | |
| ATACAGCCTATGGAAGCCAGAAGATAGTGGGATGACATTTTTGGAGTACTAG | |
| TAGAAATGTCATATGAAGAACTCTGTAGGAATGTAACATACGGTCCCATATA | |
| TGAAGCTCCTGGGTCAAGTATACCTGAACATAATTCAGGGATTTGAGGGACT | |
| TTCTTGTAACCTGAGGATCAAGATGTCAAGGAATTAAAAACATGTATAAAAC | |
| ATTGTTGTATAAAAACCCATTAAAAAGAATGGAAGACACTATAGTAAAATCA | |
| TTGTGGGTTTAGTTGTTATAACACATTTTAAAAATCTTTGATCCCAATCAAT | |
| ATTTATAAGAAAGAAGAAATATGGAATTATTTCCTGAGTCAAGGAGCAGGGA | |
| GAGAATGAGGAAGAAGAGGAGGAGGAGGAGGGGGAGGAGGAGACAATAAACC | |
| TACTTCCCAAAGTTAACAAACAAAAAGTGGGAAGAGGTCAAAGACTACAAGG | |
| AGTAGAATTAACGTCAATTGTTTCTATGTTTGAGTCTGAAAATTTTTTGTCC | |
| CTTCTCCACCAACCTATATATTGATACACATATAAATGCTAAAGGCATTTTT | |
| GAATTTGAACAGATCATTTTCTTTGTATGGCTGCCTTTAAAAAAAATTCAAC | |
| CTGGTCACTCTTCCTCAACATTTACTGAGGTCTAAGTGTTCAATTTAGAACA | |
| CATGCTTTAATAACTCAGAGACCTGTCATTTGTCACAAATCTTGCCTAGAGA | |
| AATACTCATTAGCGAATTAGGCAGAAAGAGGATGCAAAATAAAAAGGCACAG | |
| TAGTCCCCTGATATCCATGGAAGACTGGTTCCAGGACACCACCAAACCCCTC | |
| CCCGCAAATACCAAAATCCATGGATGTTCAAGTTTCTTAACATATCATGGCA | |
| TAGTATTTGCATTTAACCTACACACATCCTCTTGTACACTTGAAATTATCTT | |
| TAGATTATTTATAATACTTAATAGAATGTAAATGCTATGTAACTAGTTGTGT | |
| ATCATTTAGGAAATGATCACAAGAAAAAAAGTCTACAGATGTTAGTCCAGAC | |
| ACAGCCATCCTTTTTTTTTTTTTCAAATATTTTTGATCTGTGGTTCATTGCA | |
| TCCACAGATGTGGAACCCATGGATACTGTGGGCTAACTGTATTAATAAAAAA | |
| GTGGAAACATCCTAAGTTTCATGGGTGTTTAAATTGGTCAGCAACTTCCTTC | |
| TGAAGAAGTATCAGAATTTGTGAGCAATGTTAATATTTTTGTTTTCTCACTA | |
| AGAGCCACAGTTCTGAATAGAGGTTTTTAAAAAGCCCTAGCAAGGTTTCTTT | |
| AGCAATGAAACTAACATTTAACTGTATCATCAGCTTCGTGTTACATCTCTTT | |
| CCTGACTGTTGGGTGAGCCCTCCTCGGATGCTTGCTTCTGGCTACACGCCCC | |
| TTTACCCTTTTCTCTGCACTGTTTTCATCTTTATAAAGTCAGAGTTGGTGTC | |
| TATAGGCTCTCTACTGCCACATTCAAGACCTGCCTCGCTCAATGTCACCTTC | |
| AAGATGCAGAAATAGGGATTTGGGAAGGGGATTGTGAAATTTTCGAAGTCTT | |
| CCAAAATACTTTGAGAAACTATATTTGGAAGACTTTGGGGGGAGAGGTTGGA | |
| CAGGAAGGGTCTTCAGAGATCATCAAATTTAACTTTCTAAATCCTAAGGAGG | |
| AAACCGAGACTCCAGGATGTGAAGTCCCTTCTCTACCAAACTAGAATGGATG | |
| CAGGAGGAATGTCTGAGGTGCAATCCTTATCCTTTAGCAAAGGTGTCCTCTG | |
| CGTCTTCTTTAACCCATCTCTTGGACCTCCAGAAAGACAGCTGAGGATGGCA | |
| AGGGGAGTCTGGAACCACTGGAGTAGCCCCCAGCCTCCTCCTTGGAGGGCCC | |
| CCATGAAGGAGGCCCTTCAGTGACAGAGATTGAGAGAGAGGGAGGGCGAAAG | |
| GAAGGAAGGGGAGCCAGAGGTGGGAGTGGAAGAGGCAGCCTCGCCTGGGGCT | |
| GATTGGCTCCCGAGGCCAGGGCTCTCCAAGCGGTTTATAAGAGTTGGGGCTG | |
| CCGGGCGCCCTGCCCGCTCGCCCGCGCGCCCCAGGAGCCAAAGCCGGGCTCC | |
| AAGTCGGCGCCCCACGTCGAGGCTCCGCCGCAGCCTCCGGAGTTGGCCGCAG | |
| ACAAGAAGGGGAGGGAGCGGGAGAGGGAGGAGAGCTCCGAAGCGAGAGGGCC | |
| GAGCGCCATG | |
| Parathyroid hormone promoter sequence: | |
| AGATGAGGAAACTGAGGTCCAGACAGCCGAAGAGTGGTAGTGTCCAGGACAC | (SEQ ID No.10) |
| ACAACTGGTAAGCGGGCAAGCACAGGCTGTTGCTTAGCCCAGACTCATTTCC | |
| CAGGGCCTCATGCATTCGCTTCCTOCGCGATCCTTAAAGCCCTGCGCTCCAG | |
| GCATCCCCAGCCCCTCCCTCTGCCTCAGTTTCCCCACTTGGTACCGGGAGGT | |
| GGTAGGTTTGGGGTCGAAGGGCCCCTCCTCTTAGAGCTCCAGCGTGCCCTCC | |
| CCAGCCAAACACAGAAATCCCGCCCCGTTCAGCCCCAACCCCCGCGGACTCC | |
| TCCTTGCCTTCCCCTAAGTCGAGGGTCCCAGGCGGCCCGGTCCGAGCCGGCC | |
| GATAGCTTTTGGGAGTGGGGGTGGGAACGGGGGAGGGAGGTGAAGCCTGAGA | |
| GTGGGTGTCTGGATTGAGCCCCAGGTCTGGCAGCCTCGAGCCTCCGGGGTTG | |
| GGGCTGGGCAAGCTGGAGAGGCCCGGCCAGCAGCTGAATGGGTCGAGACTCG | |
| GAGACCCGGACCCGAAGAGACGCTGGGCAGGGAGGGAGCGGGATGTGTGGCT | |
| GCAGACCTGGGCGGGGGTCGGGGCTGGCCTAGGGCCGAGAGGAACGACAGGC | |
| CTGGGATGGGACTGAGGGCAGGGGACGAGGCGAGGGTGGGGCTGGACGTGGG | |
| GGAGGGCGGCAGCAGCCAAGCCGGGCTCGGGGCTGGCAGCCGAGCGGCCTCC | |
| CCAGGGACCCCGACCCGGCCCGAACGGGAGCCCAGTGGACTGACAGCGTCGC | |
| GGCCGGGGGCGCGCGGGGGTACCGGGCAGCCTCCTCAGGGGATTCGCCCATG | |
| ATGAAAGAGGGCTCGCTTCTCGGCTCAGGGTCTCTATTCGCCAGCGGGGGCC | |
| GGATGATCAAGGGAAAAAAAATTTAAAAGCCCGTGCTTTCCAGAAGAGAATG | |
| AAGCGGCGGCGGCGTCCCGGGTTCCCTGCTCGGGTCTCGATGTTACAGCTGC | |
| CCCCGCCCCGTCTCCCCAGCACTCACATCCCGCCGCCGTAAGACTCCGGGCC | |
| TCGGCCTCTAGCGCAATGTCCCGGGGCGGGGGGCGGAAGGCTCCTCTCGGCC | |
| TCTCCACACTCCCGCGTCGGCGGCTGCGGAGGGGGTGGGGGCGGGAGAGGCC | |
| CGGGAGGGCGCGGGGAGGGAAGAGGCGCCCGGCCGGGGAGAAGGGGGAGCGG | |
| CAGACGCCGAGGCGAGGGATGCGCGCGGCGGGCGGTGGCTCCGAGCGGCGGC | |
| CGGGCGGGGGGCGCTGGAGGCCAGGCCGGCCAGCGGGGGGTATCCCGAGAGC | |
| TCCATGAAGTCCCCCCGGGGCCGCGGACGGGGCGCTGGCTTGGGGAGGCTGT | |
| CGGGGGGGCCCCGACATCCATGGCAAGGCGGGGGCCGCGGCGGCGCGCTCGG | |
| AGTAAGTCGGGGCTGGGGACCCGCGCCGAGGGGAAGTGGCCGGAGTCGGGGA | |
| GGAGCGACTCCGGGCCTGGCCGGAGCAGCCAGGCTGCTCTGTCTCGGTGTCA | |
| GTCGGCGGCGCCTCCTCGGAACCCGGGGGAGTCGCCAGCCCCGCGCCGCTCG | |
| GCTCGGTGGCTTTTTTGGAAACTTGCAAATGTTTTCGTAGAGAGAAAAGGGG | |
| GAGGGAGGGAGCGAGGGAGTGACCGAAACGGAGCTTGGGGCCGCTGGAAGAA | |
| CTGAGGCCAAGGCCGGGGGAGCTAGAGACGGACTGACAGACAGGCAGACCGA | |
| CAGAGCGTCGGGGCCGCTGCGCGCCCGAGCGGCACAGGCGCAAGCGGGGCTC | |
| TGGCCAAGGATGGGGAAGGGGTGCGGGAGGCGGCTGCCGAGGGTCTGGGATC | |
| TCAGGAGGCCGAACGGCCGGGGGCTGGCGGCCGGAACACCTAAGGGCTCAGT | |
| GTGGCTGCAAAGTTGAGATCGCACCCCCTAACTGCACGCCCCGCGCGGCTCA | |
| GAACGCGCCCCCTGCCCGGCCCTGACTCCCTACGCCGAAAGTCGCGGAGCTA | |
| AAAATAACAGTCCTGCGCGCCCCCCGCAGACCGCGACCCCGACCCCTCCCCC | |
| GCCCCCTCCCCCCACTGGGCGTGGGGCGAAGCCACAGCTCCCATTTCCCCAA | |
| AAGAAAAAAAAAGAAAGAAAGAAAGAAAGAAAGAAAGAAAAGGCGGCGCGGG | |
| AGGGGGGCGGGGGGCGGGCCGGGGGAGGCGGGCCCGGCCATATGGATGTGAT | |
| TTCTTCGCTCCGAGGCAGACGGGCCGCTCCGCAGCGCTCGGCGCCCGCCCGC | |
| CGCCCGCCCGGCCTCCGGCTCTCCCTCCCTCCCTCCTGTCCCTCCCTCCCTC | |
| CCTCCTTTGCGCTGCTCGCTCGCTCGCTCGCTCGCTCGCCCTCAGCGCATGG | |
| GCCCCGCGCCGGGCCCCGGGGCCTCGGGCCGCCGGGACGCCGGGGTCCCATA | |
| GGCCGGGGCGTGGGCGGGGCGGCCAGCCTGACGCAGCTCTGCACCCCCTACC | |
| ACCCCAGGGCCGGCGGCGGCGGCTGCCCCGAGGGACGCGGCCCTAGGCGGTG | |
| GCG | |
| Calcitonin receptor promoter sequence: | |
| ATATTAGGGTGTCGATTTGAGATCTTTGCAGCTTTGTGATGTGTGCATTTAG | (SEQ ID No.11) |
| TGCTATAAATTTCCCTCTTAACACTGCTTTAACTGTGTCCCAGAGATTCTGG | |
| TACATTGTCTCTTTGTTCTCATTGGTTTCCAAGAACTTCTTGATTTCTGCCT | |
| GAATTTTTTTAGTCCTGAGTTCTAATTTGATTGCATTGTGGTCTGAGAGACT | |
| GTTTGTTATGATTTTAGTTCTTTTGCTTTTGCTGAGGAATGTTTTACTTCCA | |
| ATTATGTGGTCGATTTTAGAATAAGTTCCATGTGGTACTGAGAAGAATGTAT | |
| ATTCTGTTGATTTGGGTTGGAGAGTTCTGTAGATGTCTATTAGGTCCACTTG | |
| ATACAGAGCTGAGTTCAAGCCCTGAATATCCTTGCTAATTTTCTGTCTCATT | |
| GATCCTCTCTAATATTGGTAGTAGAATGTTAAAGTCTCCCACTATTATTGTG | |
| TGGGAGTCTGAGTATCTTTGTAAGTCTCTAAGAACTTATTTTATGAATCTGG | |
| GTGCTCCTGTATAGGGTGCATATATATTTAGAGTAGTTAGCTCTTGTTGAAC | |
| TGTTCCCTTTACCATCATGCAAGGCCTTCTTTGTCTTTTTTTTTATCTTGTT | |
| GGTTTAAAGTCTGTTTTGTCAGAGACTAGGATTGCAACCCATGCTTTTTTTT | |
| TTTTTTTTTTTCTTTCCATTTGCTTGGTAAATTTTCCTCCATCCCTTTGTTT | |
| TGAACCTATGTGTGTCTTTGCACATGAAATGGATCTCCTGAATATAGCACAT | |
| CAATGGGTCCTGACTTTTTATTCAATTTGCCAGTCTGTGTCTTTTAATTGGG | |
| CCATTTAGCCCATTTACATTTAAGGTTAGCATTCTTATGTGTGAATTTGATC | |
| CATCATCATGATGCTATCTGGTTATTTTGCACAACAGTTGATGCAGTTTCTA | |
| CATAGTGCCATTGGTTTTATATTTTGGTGTGTTTTTGCAGTGGCTGGTACTG | |
| GTTTTTCCTTTCCATATTTAGTGCTTCTTTCAGGAGCTCTTGCAAGGCAGAC | |
| CAAATGGTAACAAAATCTCTCAGCATTTGCTTGCCCAGAAATGATTTTATTT | |
| CTTCTTCGCTTATGAAGCTTAGTTTGGCTGAATATTAAATTCTGGGTTGAAA | |
| ATTCTTTTCTTTAAGAATGTTGAATATTGGCCTCCAATCTCTTCTAGCTTGT | |
| AGAGTTTCTGTTGAGAGGTCTTCTGTTAGTCTGAAGGGCTTTGCTTTGTAGG | |
| TTACTTTGCCTTTCTCTCTGGCTGCCCTTAATATTTTTTCATTCATTTCAAC | |
| CTTGGAGAATCTGATGATTATGTGTCTTGGGGTTGATCTTCTCATGAAATAT | |
| CTTAGTGGTGTTCTCTGTATTTCCTGAATTTGCATGTTGGCCAGTCTTGCTA | |
| TGTTGGGGAAGTTCTCCTGGATAAAGGATAGGTAAATTCTATGGGTAATACA | |
| GTAGATATAGTGCAACAGGAACTTACCAGTTAAGATACAGTCATAACCACTC | |
| ACCCCTAGTTGGAATGTAGGTTTCACACAACTCCCACTGATGAAAAGAAATA | |
| TATGTATTTTTCAACTGTTTAACCCTTTGTTAAGTTTTCTTGTGTAAAATTA | |
| TCTGCAGAGCCATGAAAAACCATTTGATATTTGTGACTAAGCAGCCTGTTTG | |
| GATGATTATGCTCTTCAGTATGAATGGTGAGCTGTTAAATGACATGCTCAAT | |
| CATTGCTATGGAAGAAATTTGTTCTTACTAGCAACTTGAAGCTTAAAGAAAC | |
| ATTTATAGGAAAGAAAATTACTCAAAGCTTTAAATAAGGCTACTTTTAGAGT | |
| TGGCCTTAGACTACCTAGAGGGCATGATGATTAATCTTTCACAAATTACAGA | |
| TTTTATTTGTTCATGTCCAGTGAGGTGACTTCTTGGTGGACATCTTCATTGC | |
| AATTTTCAGCAGCTCTATCAATGACACATGTTAACTGAAGCTGACATGGGTT | |
| GCTCTTGCTCTCTTGGAATGTCTTTATTTCTGTCCTAATATGCAAAGGTAGT | |
| GCCAGAATTTCTTAATAGGAGGGCCTCAGGTATAACAATCTAGTTGACAGGA | |
| AAAGCAATGGAATCTTCACTGCATTTGCATCACAAGCATACTGTTTTTTCTT | |
| ACGTGTGTTTTTTAGGGTGTCTTGGGATGTTGATCCTCTTTAAGTCAAATAG | |
| AAAAAATGAAAATGAAATGCCATAGCCAATATTAGAGATATATTAATTTTAG | |
| TCTTTGTTGCTTTTATATTTTTCTAGGACAAAGAGATCTTCAAAAATCAAAA | |
| BMP2 Promoter: | |
| GAAAAACTTTGAATGGACCTTTGAAAACGGTAGAATTGACAATGGTTAGCTG | (SEQ ID No.12) |
| CAAGTGATATTTTCAAGGCAAACAGACACTCTCCCAAAGTATTAAATAACCC | |
| AGCATTCTAAGTTGCAGGTGGAAGGTAGCCATTAGTGAAGAGAGAGAAAAAA | |
| AAAAAGAAATAGCTCGTCTGTATTTAGATTTATCATTTCTGACTATTGCTCT | |
| TCCCTGGAAAACGGGTAGGTACAGTCATCCTGTACTTCGATCCCAAATCAGT | |
| CTCTGGAGACTACTTATTTATTTATTTATTTATTTATGGACTTCTTTCTTTC | |
| AAGCGTTCGAACTCATTTCCACCACAAGAGGGCAGCCATCTCTAAAAAAAAA | |
| AAAATAGGGCCAAAATTTATGTAAGTTGTGCTTGGAACAAGCATTCAGTAGT | |
| TCCTCAGAAATCATACACCCTACATAAAAGAGATTCTGCAATGGGCAGCACT | |
| AACATGAAACAGTGTTCAGAAGTACCCATTTTCCCTCAGATTCTAAACTGAC | |
| AAGGTTTCCACTTATCAGGTTATGAAGTTCTAAAGCTGCAAGACATCCTTGA | |
| GGTCATCACAGGATATTTATTTATTTTTTCTTCGGGTGCATCCAATAGTTAT | |
| CAACTTTTCCTCCTCTTTAAAAGCTACTTAAATCTCATTGAAGTTTTGTTTT | |
| GTTTTGTTTTTGAAATCTAAGTAATGAGAGAAACAATTGTTAACTTCTCAAT | |
| TAAACTTGATAGGAAAGGAAATAATTTCAGAAGCCCTGTGTCCATGAGTAGG | |
| ATATGTTTTATTGCCTCCTTGTTTGCGGTGCAATGACTCTGAGTGACAATCA | |
| ACTTCTATAGCACCTTTTTTTTTTTTTTTTCAGGAAATAAAGTAGCATGTTC | |
| CTGAATAATTCCCCCACCCCCTTTTATTTTCCTGGTAGTCAGGCTTCCTCCA | |
| AAATACCTTATTTGACCTTTATACCTTTAGAAACAGCAAGTGCCTAATTCGC | |
| CTCTGTGGGTTGCTAATCCGATTTACGTGAGCGGAACCTAGTATTATTTTAG | |
| CTCCCCTACCGAAAAAATAATACACATGGATAATAGTTCTATTACCAGCTCC | |
| TGCTTCTGACTTTTTTCTCTCTGTTTCGCAGGCCCGATAGCTCTGGGAAAGC | |
| AGAACTTGGCCTTTTCCAAAAATTTTCTGCCCTTGGTTTTGGGGATCATTTG | |
| GGCAAGCCCGAGGTGCTGTGCATGGGGGCTCCTGGAATCCTGGGAAGGGCAG | |
| AAAGCCTTGGCCCCAGACTCATCGTGCAGCAGCTCTGAGCAGTATTTCGGCT | |
| GAGGAGTGACTTCAGTGAATATTCAGCTGAGGAGTGACTTGGCCACGTGTCA | |
| CAGCCCTACTTCTTGGGGGCCTGGTGGAAGAGGGTGGCGTAGAAGGTTCCAA | |
| GGTCCCAAACTGGAATTGTCCTGTATGCTTGGTTCACACAGTGCGTTATTTT | |
| ACCTTCCTCTGAGCTGCTAATCGCCTGCCTCTGAGCTGGGTGAGATAAATAT | |
| CACAAGGCACAAAGTGATTGTACAATAAAAAAATCAAATCCCTCCCATCCAT | |
| CCTTCAGTCTGCCACACACGCAGTCTACGTTACACACATGTCACGTAAAGCA | |
| GGATGACATCCATGTCACATACATAGACATATTAACCGAAATGTGGCCCTTC | |
| GGTTGCATATATTCTCATACATGAATATATTTATAGAAATATATGCACATAT | |
| TTTTGTATATTGGATATATTTATGTAACTATAAATTTACATGCGTATGGATA | |
| TGAAAATAAATGCATACACATTTATGTAAAAAAATTTGTACACATGCATTTA | |
| CATATGTAAATACATACATCTCTATGTATTAATGTTTAAAAACACTCAATTT | |
| CCAGCCTGCTGTTTTCTTTTAATTTTCCTCCTATTCCGGGGAAACAGAAGCG | |
| TGGATCCCACGTCTATGCTATGCCAAAATACGCTGTAATTGAGGTGTTTTGT | |
| TTTGTTTTGTTTTTTGAAATCGTATATTACCGAAAAACTTCAAACTGAAAGT | |
| TGAATAACGGGCCCAGCGGGGAAATAAGAGGCCAGACCCTGACCCTGCATTT | |
| GTCCTGGATTTCGCCTCCAGAGTCCCCGCGAGGGTCCGGCGCGCCAGCTGAT | |
| CTCTCCTTTGAGAGCAGGGAGTGGAGGCGCGAGCGCCCCCCTTGGCGGCCGC | |
| GCGCCCCCGCCCTCCGCCCCACCCCGCCGCGGCTGCCCGGGCGCGCCGTCCA | |
| CACCCCTGCGCGCAGCTCCCGCCCGCTCGGGGATCCCCGGCGAGCCGCGCCG | |
| CGAAGGGGGAGGTGTTCGGCCGCGGCCGGGAGGGAGCCGGCAGGCGGCGTCC | |
| CCTTTAAAAGCCGCGAGCGCCGCGCCACGGCGCCGCCGCCGCCGTCGCCGCC | |
| GCCGGAGTCCTCGCCCCGCCGCGCTGCGCCCGGCTCGCGCTGCGCTAGTCGC | |
| TCCGCTTCCCACACCCCGCCGGGGACTGGCA | |

In order to isolate the DNA encoding the promoter region, BAC clones with the desired sequence or genomic DNA preparations from source cells were used. This DNA can be used as a template for polymerase chain reaction (PCR) amplification of desired sequence with primers designed specifically for the sequence. These primers can or can not contain restriction enzyme cleavage sites to facilitate cloning into the reporter gene construct. The amplified DNA sequence is cloned into a reporter gene construct by standard molecular biological techniques.

Genomic DNA was purchased from a commercial source and used as template for PCR. The following primers were used to amplify the indicated sequences:

EXAMPLE 3
Forward primers 5′ --> 3′

| Promoter | | | | restriction |
|---|---|---|---|---|
| set # | Gene | Primer | Tm | site |
| | CBFA-1 | AGTCGAATTCTATTGTGATCTAATA | 47.856287 | EcoR1 |
| | | TGAACCAAAA (SEQ ID No. 13) | | |
| | MMP9 | AGTCCTCGAGGGCTTATAGAGAACT | 50.257868 | Xho1 |
| | | TATTACGGTG (SEQ ID No. 14) | | |
| | Osteo- | AGTCGAATTCAAAATAGGTTAGGCA | 50.184913 | EcoR1 |
| | protogerin | ACTAGTCTGA (SEQ ID No. 15) | | |
| Hs.194236 | Leptin | AGTCAAGCTTAGTAAAGTATTTATT | 47.252718 | HindIII |
| | | CTAGATGGCC (SEQ ID No. 16) | | |
| Hs.166015 | FGF6 | AGTCCTCGAGCCGTGGTGACAGTAG | 60.502523 | Xho1 |
| | | GAACAAGTGG (SEQ ID No. 17) | | |
| Hs.170195 | BMP7 | AGTCCTCGAGCTGCCCAGCATGGTG | 60.943072 | Xho1 |
| | | CTTGG (SEQ ID No. 18) | | |
| Hs.158317 | BMP10 | AGTCCCGCGGGTTGACATCTGTGTG | 54.028311 | SacII |
| | | TGTGTGAAGA (SEQ ID No. 19) | | |
| Hs.2534 | BMPR1A | AGTCCTCGAGAATCCATCTATTTTA | 43.700475 | Xho1 |
| | | CTCTTTATAA (SEQ ID No. 20) | | |
| Hs.115770 | Rank ligand | AGTCCTCGAGGTATTTACCATGCAC | 48.744767 | Xho1 |
| | | CTACTATAGC (SEQ ID No. 21) | | |
| Hs.37045 | Parathyroid | AGTCGAATTCAGATGAGGAAACTG | 57.080977 | EcoR1 |
| | hormone | AGGTCCAGACA (SEQ ID No. 22) | | |
| Hs.640 | CalcR | AGTCGAATTCATATTAGGGTGTCG | 51.546295 | EcoR1 |
| | | ATTTGAGATCT (SEQ ID No. 23) | | |
| | BMP2 | AGTCGAATTCGAAAAACTTTGAAT | 54.847755 | EcoR1 |
| | | GGACCTTTGAA (SEQ ID No. 24) | | |

Reverse primers 5′ --> 3′

| | | | | |
|---|---|---|---|---|
| | CBFA- 1 | AGTCACGCGTAGTCCCTCCTTTTTT | 52.924004 | Mlu1 |
| | | TTTCAGATAG (SEQ ID No. 25) | | |
| | MMP9 | AGTCAAGCTTGGTGAGGGCAGAGGT | 60.850839 | HindIII |
| | | GTCTGACTG (SEQ ID No. 26) | | |
| | Osteo- | AGTCACGCGTTGTGGTCCCCGGAA | 60.503933 | Mlu1 |
| | protogerin | ACCTCAG (SEQ ID No. 27) | | |
| Hs.194236 | Leptin | AGTCACGCGTTTTCCTTCCCAGGA | 60.075741 | Mlu1 |
| | | TGGGCTTC (SEQ ID No. 28) | | |
| Hs.166015 | FGF6 | AGTCAAGCTTAGTGATGAACAGTT | 57.375174 | HindIII |
| | | TCTGTCCCAGG (SEQ ID No. 29) | | |
| Hs.170195 | BMP7 | AGTCAAGCTTCGCGCCGGCTCTACG | 63.367187 | HindIII |
| | | CGCTA (SEQ ID No. 30) | | |
| Hs.158317 | BMP10 | AGTCGAATTCGACTCCGCTCGAGC | 60.417825 | EcoRI |
| | | TCCTAGGC (SEQ ID No. 31) | | |
| Hs.2534 | BMPR1A | AGTCAAGCTTTGTTCAATTGTAAT | 52.338666 | HindIII |
| | | GTTTCCTGTGT (SEQ ID No. 32) | | |
| Hs.115770 | Rank ligand | AGTCAAGCTTGGCGCTCGGCCCTC | 64.782309 | HindIII |
| | | TCGC (SEQ ID No. 33) | | |
| Hs.37045 | Parathyroid | AGTCACGCGTCGCCACCGCCTAGG | 65.161157 | HindIII |
| | hormone | GCCG (SEQ ID No. 34) | | |
| Hs.640 | CalcR | AGTCACGCGTTTTTGATTTTTGAA | 51.546295 | Mlu1 |
| | | GATCTCTTTGT (SEQ ID No. 35) | | |
| | BMP2 | AGTCACGCGTTGCCAGTCCCCGGC | 67.64611 | Mlu1 |
| | | GGGG (SEQ ID No. 36) | | |

Vectors for Delivery of Reporter Gene Constructs Into Cells

pXI Retroviral Vector

The pXI retroviral vector provided herein delivers high-titer retroviral production, and ubiquitous and high-level gene expression in target cells. It has further optimized to facilitate image-based cDNA matrix-based expression screening. Schematically the vector contains the following elements: hCMV-R-U5 - - - psi - - - sp6 - - - attR1 - - - CmR - - - ccDB-attR2-T7 - - - SV40 - - - AsRed - - - nu c - - - sCMV-R-U5

Elements

The 5′ LTR (hCMV-R-U5) of the pXI vector contains sequences from the human CMV (hCMV) promoter, which replaces the 5′ U3 region of the Moloney LTR to provide high expression of the retroviral RNA in packaging cells. The R, U5, and psi sequences required for reverse transcription and packaging have been retained in the vector.

GATEWAY™ cloning cassette (Life Technologies; see Life Technologies GEN 20:44; sp6-attR2 - - - CmR - - - ccDB-attR2-T7, from pDEST12.2 (see SEQ ID No. 37; available from Invitrogen, Life Technologies, Carlsbad Calif.) is downstream from 5′LTR sequence to accept cDNA from GATEWAY™ adapted plasmids and libraries. The GATEWAY™ cloning sites (attR1 and attR2) are flanked by sp6 and t7 promoter sequences to facilitate rapid sequencing of cDNA insert. Plasmid pDEST12.2 (SEQUENCE ID NO. 37) is 7278 bps DNA circular vector with the following features:

| Start | End | Name | Description |
|---|---|---|---|
| 15 | 537 | CMV | promoter |
| 687 | | SP6 | promoter |
| 730 | 854 | attR1 | |
| 963 | 1622 | Cmr | Chloramphenicol resistance |
| 1742 | 1826 | ccdA | ccdA inactivated by cutting at Nde I, filling, and |
| | | | ligating closed. |
| 1964 | 2269 | ccdB | |
| 2310 | 2434 | attR2 | |
| 2484 | | T7 | promoter |
| 2619 | 2981 | SV40 | small t-intron & polyadenylation signal |
| 3175 | 3631 | f1 | intergenic region |
| 3695 | 4113 | SV40 | ori & early promoter |
| 4158 | 4952 | Neor | Neomycin resistance |
| 5016 | 5064 | poly A | synthetic polyadenylation signal |
| 5475 | 6335 | Apr | Ampiciilin resistance |
| 6484 | 7123 | pUC | ori. |

An SV40-AsRed expression cassette (SV40 - - - AsRed-nuc) is downstream of the GATEWAY™ sites. Expression of the AsRed florescent protein (Clontech) ‘marks’ cells that have been transduced with the retrovirus during image analysis of expression-based assays. The AsRed protein has been modified to localize to the nucleus.

The 3′LTR (sCMV-R-U5) of the pXI vector contains sequences from the simian CMV promoter (sCMV), and upon reverse transcription of the retrovirus, will d3rive high level expression of the inserted cDNA. Furthermore, since the hCMV and sCMV share very little sequence homology, the risk of recombination during pXl plasmid amplification is greatly reduced. R and U5 regions from MLV are downstream of this promoter sequence.

### EXAMPLE 4

### Generation of Viral Particles and Cells Containing the Reporter Gene Constructs

This example demonstrates of preparation of responder cells by transient and stable transfection and use of the cells. The following e method was used to generate a robust reporter gene assay for inducers of the ABC1 (ATP-binding cassette 1) transporter promoter, which controls the cellular apoliprotein-mediated lipid removal pathway.

Vector Construction

A region of 1033 bp in the proposed promoter of Homo sapiens ATP binding cassette transporter 1 (ABC1) was PCR amplified from the genomic DNA extracted from 293 cells using DNeasy Tissue Kit (Qiagen, Valencia, Calif.). The sequence of the cloned ABC1 promoter correlates with bases 1-1033 of GI8677405 (Genbank). The sequences of the PCR primers were:

5′-GCGCGGCAACGCGTATAAGTTGGAGGTCTGGAGTGGCTA-3′ (SEQ ID No. 41) and 5′-GCTAGGAAGCTTGCTCTGTTGGTGCGCGGAGCT-3′ (SEQ ID No. 42). The amplified promoter was cloned into the Mlu I and Hind III sites of the vector pNFκB-Luc (Clontech; see SEQ ID No. 44). The resulting vector was termed MAL. Sequencing of MAL using primer pairs F1(5′-GCGTATAAGTTGGAGGTCTG-3′; (SEQ ID No. 43) and R1(5′-GACTCTCTAGTCCACGTTCC-3′; (SEQ ID No. 38), F2(5′GGCTGAGGAAACTAACAAAG-3′; (SEQ ID No. 39) and R2(5′GTGGCTTTACCAACAGTAC C-3′; (SEQ ID No. 40) revealed a G_C mutation at position 849.

The ABC1 promoter and luciferase gene were then cloned into various retroviral vectors SIN vectors.

Establishing Stable Cell Lines Through Transient Transfection

Mouse macrophage cell line RAW264.7 from the ATCC was used for reporter gene assays. RAW cells were cultured at 37° C. in Dulbecco's modified Eagle medium (GibcoBRL), supplemented with 10% defined fetal bovine serum (low endotoxin, Hyclone). Transient transfection was carried out in 6 well plates with SuperFect Transfection Reagent (Qiagen) using the protocol provided by the supplier. In brief, 6×10⁵ cells were seeded in each well the day before transfection. 2 μg of DNA and 10 μl of SuperFect reagent were added to the cells. For the purposed of selecting stable cell lines, vectors containing antibiotic resistant genes (e.g. hygromycin, puromycin and blasticidin) were also included at a ratio of 1:5 or 1:10 to the reporter DNA. 48 hours post-transfection, the cells were transferred into 10 cm dishes. An antibiotic was added at 150 μg/ml of hygromycin, 400 ng/ml of puromycin, or 3 μg/ml of blasticidin. Massive cell death was observed within 3 days in hygromycin and blasticidin, but not in puromycin. Two weeks later, the cells which sustained antibiotic selection were seeded into three 96 well plates at the density of 0.3 cell/well. After 3-4 weeks, 44 single clones each of MALH (hygromycin) and MALB (blasticidin) were harvested and assayed. Pools of MALH or MALB were also combined for population experiments. The total selection time was 5-6 weeks.

Establishing Stable Cell Lines Through Retroviral Transduction

Day 1: HEK293 cells were seeded at 8×10⁵ cells/well in 6 well plates. 3×10⁶ RAW cells were seeded in a 10 cm dish.

Day 2: HEK293 cells were transiently transfected with a cocktail of 2.5 μg reporter vector and retroviral packaging plasmids; 2.5 μg Gag-Pol vector and 2.5 μg VSV-G expression vector using CalPhos Mammalian Transfection Kit (Clontech) in the presence of 50 μM chloroquine. The transfection medium was replaced with fresh growth medium 6-8 hours after transfection.

Day 3:24 hours after transfection, the medium containing retroviral vector was collected and replaced with fresh medium for RAW cells. RAW cells were seeded in a 6 well plate at 6×10⁵ cells/well.

Day 4: The second batch of retroviral vector containing medium was collected, filtered through 0.45 um filter, and used to infect the RAW cells in the presence of 5 ug/ml protamine sulfate.

Day 5: The transduced cells were changed into fresh medium 16 hours after infection.

Day 6: The transduced RAW cells were transferred to 10 cm dishes. In needs of antibiotic selection (for SAILN and SAILpANeo), Geneticin (50 mg/ml, Gibco BRL) was added to the cells at a final concentration of 800 ug/ml. The cells were maintained in G418 for a minimum of 4-5 days and then assayed. Total time to derive stable populations was 1 week (3 days if no selection was used).

Reporter Gene Assays in 96 Well Plates

Day 1: RAW cells were seeded in 100 μl growth medium at 2×10⁴ cells/well in 96 well white plates with clear bottom.

Day 2: The cells were changed into BSA medium. The BSA medium contains Dulbecco's modified Eagle medium supplemented with penicillin, streptomycin, L-glutamine, and 2 ug/ml fatty acid free bovine serum albumin (Sigma). The cells were stimulated with a final concentration of 10 uM 22(R) hydroxycholesterol, 10 uM 9-cis retinoid acid, or a combination thereof. Both compounds were pre-dissolved in ethanol at the concentration of 10 mM. Day 3:24 hours after induction, the cells were assayed with Bright-Glo Luciferase Assay Reagent (Promega) at room temperature. With a 15 min incubation time, the plate was read with LJL Acquest with an integration time of 0.1 sec per well.

Screen for 10,000 Compounds

Day 1: RAW cells were seeded in five 10 cm dishes at 3 million cells per dish.

Day 3: RAW cells were harvested. 108 million cells were spun down and diluted into 180 ml BSA medium at a density of 6×10⁵ cells/ml. Using Cartesian, the cells (4×45 ml in 50 ml corning tubes) were plated into twenty 1536 well plates at 5 μl per well, resulting in 3000 cells/well. Eighteen of these plates were used to screen for ˜11000 compounds from the collection of compound libraries. This process took 90 min.

Day 4:20 hrs after plating the cells, 50 nl of 1 mM 22(R) hydroxycholesterol in ethanol was added to each well of 9 plates. Then 50 nl each of the compounds to be tested were added to the cells, giving a final concentration of 10 uM compound and 1% DMS0. With 20 min per plate, this step took ˜6 hr.

Day 5:24 hrs after adding the compounds, cells were assayed. 5 μl of Bright-Glo was added to each well using Cartesian (4 min per plate). After 13 min incubation, the plate was read with Acquest (6.5 min per plate). In combinations, it took 20 min per plate and 6 hr for the whole assay.

The following studies were done to test demonstrate the utility of the SIN retroviral vector system for rapid assay development. Populations of RAW cells with stably integrated forms of the ABC promoter construct generated by different methods were tested for their inducibility.

The stable transfection approach resulted in populations MALH and MALB. Forty-four total clones out of a starting population of 1.2×10⁶ RAW cells survived selection, 10 (5 from MALH and 5 from MALB) of which were inducible by HCh (hydroxycholesterol) and RA (retinoic acid). The calculated efficiency of stable cell line generation was 0.0037%. Stimulation of the 44 clones together yielded a net 1.5-fold increase in luciferase activity versus unstimulated. Stimulation of combinations of the 5 inducible MALH clones or the 5 MALB clones resulted in 3.9 and 7.6-fold induction respectively.

The retroviral transduction method resulted in 5 independent populations of RAW reporter cells. SAIL, SALG and SAILG populations were generated in 3 days total and immediately tested. Upon stimulation with HCh and RA, the respective fold-induction was 8.3, 14.7 and 2.9. All but the latter population yielded as good or greater induction than the stably transfected populations. The low induction in SAILG cells can be experimental error, lower viral titers or some other phenomenon. In the SAILpAneo and SAILN experiments, cells were selected with G418 (Geneticin) for 5 days resulting theoretically in 100% of cells encoding the reporter gene. Induction levels were 4.7 and 14.7 respectively here. The lower induction with SAILpANeo can be explained by the orientation of the promoter driving Neo expression and it's effects either on viral titers and/or ABC-1 driven transcription.

Total time to derive reporter cell lines was under 1 week in all 5 retroviral cases. Furthermore, SAILN cells were successfully adapted to industrial automation and 1536-well microplate small molecule screening. The methods are less time consuming than other methods. This collection of cells is used to assess the effects of test compounds and other perturbations on this pathway and to provide information regarding targets in the pathway of test and known perturbations.

Since modifications will be apparent to those of skill in this art, it is intended that this invention be limited only by the scope of the appended claims.

What is claimed is:

1. A method for producing a collection of responder cells, comprising:
a) obtaining an expression profile of a genome or a transcriptome exposed to a perturbation;
b) identifying genes that are differentially expressed under the perturbation compared to the absence of the perturbation;
c) identifying and isolating regulatory regions from one or more of the genes that are differentially expressed;
d) operatively linking each regulatory region to nucleic acid encoding a reporter to produce a reporter construct; and
e) introducing each reporter construct into an addressable collection to cells to produce an addressable collection of responder cells.

2. The method of claim 1, wherein a plurality of regulatory regions that respond to a perturbation are identified.

3. The method of claim 1, wherein the regulatory region comprises a promoter.

4. The method of claim 1, wherein the regulatory regions comprise robust responders.

5. The method of claim 1, wherein the perturbation comprises exposure to a test compound or plurality thereof.

6. The method of claim 5, wherein the test compound is a biopolypmer, a small organic molecule or a natural product.

7. The method of claim 6, wherein the test compound is a nucleic acid molecule or a polypeptide.

8. The method of claim 6, wherein the test compound is an antibody, a member of a combinatorial library, an antibody or binding fragment thereof, or antisense molecule.

9. The method of claim 1, wherein the genome is eukaryotic genome.

10. The method of claim 1, wherein the genome is an an animal insect, plant or yeast genome.

11. The method of claim 1, wherein the genome is a mammalian genome.

12. The method of claim 10, wherein the animal is a human.

13. The method of claim 1, wherein the transcriptome is from a tissue or organ.

14. The method of claim 1, wherein the perturbation is a disease state in the organism and expression is compared to its absence.

15. The method of claim 1, wherein the transcriptome is from a cancerous tissue or organ.

16. The method of claim 1, wherein expression of genes operatively linked to the regulatory regions is repressed and/or increased under the perturbation.

17. An addressable collection of responder cells produced by the method of claim 1, wherein the collection contains a plurality of sets of cells; and each set contains a different reporter construct.

18. The collection of claim 17, wherein each set is in a well in a high density microtiter plate.

19. The collection of claim 18, wherein the microtiter plate contains at least 384 wells.

20. A method for identifying a regulatory region of a robust responder gene among a plurality of genes comprising:
a) exposing the cell to a test perturbation;
b) determining expression of a plurality of genes in the cell in the presence of the perturbation compared to the absence thereof;
c) identifying at least one gene whose expression is increased or decreased at least 3-fold in the presence of perturbation compared to the absence thereof; and
d) identifying a regulatory region of a gene that confers increased or decreased expression in response the perturbation.

21. The method of claim 20, wherein the perturbation is a substance or change in intra-cellular or extra-cellular condition.

22. The method of claim 20, wherein at least one gene whose expression is decreased at least 6-fold in the presence of the perturbation is identified.

23. The method of claim 20, wherein the regulatory region comprises a promoter or an enhancer.

24. The method of claim 20, wherein the cell comprises a tissue or organ or a sample thereof.

25. The method of claim 20, wherein the cell is eukaryotic or prokaryotic.

26. The method of claim 20, wherein the eukaryotic cell is mammalian, insect, plant or yeast.

27. The method of claim 26, wherein the mammalian cell is human.

28. The method of claim 20, wherein the perturbation comprises exposure to a drug, a hormone, an extract, a protein, a nucleic acid, a lipid, a carbohydrate or a fat.

29. The method of claim 1, wherein the perturbation comprises exposure to a drug, a hormone, an extract, a protein, a nucleic acid, a lipid, a carbohydrate or a fat.

30. The method of claim 1, wherein the perturbation comprises increased or decreased temperature, exposure to ultraviolet light, a change in pH, a change in a salt or ion concentration, exposure to or a decrease in oxygen.

31. The method of claim 20, wherein the perturbation comprises increased or decreased temperature, exposure to ultraviolet light, a change in pH, a change in a salt or ion concentration, exposure to or a decrease in oxygen.

32. The method of claim 20, further comprising:
e) operatively linking a sequence comprising a 5′ untranslated region extending upstream of the translation initiation site of the selected gene to a reporter gene to a produce a reporter gene construct.

33. The method of claim 32, further comprising:
f) determining reporter expression in the presence of the perturbation.

34. The method of claim 32, wherein the 5′ untranslated region extends 25, 50, 75, 100, 250, 500, 1000, 2500, 5000, 7500, or 10,000 or more nucleotides upstream of the translation initiation site of the selected gene.

35. The method of claim 32, wherein the reporter gene construct comprises an expression vector.

36. The method of claim 35, wherein the expression vector comprises a viral vector.

37. The method of claim 35, wherein the viral vector is a retroviral vector.

38. The method of claim 35, wherein the viral vector contains a unidirectional transcriptional blocker.

39. The method of claim 35, wherein the viral vector contains a scaffold attachment region.

40. The method of claim 35, wherein the viral vector contains a selectable or detectable marker.

41. The method of claim 1, wherein step d) is performed by comparison of the selected gene to a sequence database containing at least one genomic sequence.

42. The method of claim 41, wherein the comparison identifies a 5′ untranslated region extending upstream of the translation initiation site of the selected gene.

43. The method of claim 42, wherein the 5′ untranslated region extends 25, 50, 75, 100, 250, 500, 1000, 2500, 5000, 7500, or 10,000 or more nucleotides upstream from the translation initiation site of the selected gene.

44. The method of claim 41, wherein the comparison is performed by a computer system or program, wherein the system or program includes computer readable instructions directing a processor to compare one or more gene sequences to a sequence database.

45. The method of claim 41, wherein the sequence database comprises a mammalian, human, yeast, drosophila, C. elegans or plant database.

46. The method of claim 41, wherein the sequence database comprises a genomic sequence database.

47. The method of claim 44, wherein the computer system or program further comprises computer readable instructions that direct a processor to select a primer set appropriate for amplification of the regulatory region.

48. The method of claim 1, further comprising ranking the genes identified in step c) according to their relative increase or decrease in expression.

49. The method of claim 48, wherein the ranking is carried out by a computer system or program comprising computer readable instructions directing a processor to rank gene expression according to increase or decrease in response to the perturbation.

50. The method of claim 1, wherein expression of a differentially expressed gene is increased to a greater extent than increased expression of one or more other genes among the plurality of genes.

51. The method of claim 1, wherein expression genes that are differentially expressed are among the top 20, 10, 5 or 2 genes whose expression is altered among a plurality of genes.

52. The method of claim 1, wherein expression of a gene that is differentially expressed is increased to a greater extent than increased expression of any other gene among a plurality of genes whose expression is increased.

53. The method of claim 1, wherein expression of a gene that is differentially expressed is decreased to a greater extent than increased expression of any other gene among a plurality of genes whose expression is decreased.

54. The method of claim 20, wherein in step c) genes whose expression is increased or decreased are among the top 20, 10, 5 or 2 genes whose expression is altered among a plurality of genes.

55. The method of claim 20, wherein in step c) a gene whose expression is increased is increased to a greater extent than increased expression of any other gene among a plurality of genes whose expression is increased.

56. The method of claim 20, wherein in step c) a gene whose expression is decreased is decreased to a greater extent than decreased expression of any other gene among a plurality of genes whose expression is decreased.

57. The method of claim 20, wherein step b) is performed by hybridization of transcripts of the genes to an array comprising a plurality of oligonucleotides at addressable loci on a substrate.

58. The method of claim 57, wherein the transcripts or nucleic acid molecules derived from the transcripts are detectably labeled.

59. The method of claim 58, wherein the label comprises a fluorophore, a radioisotope or a chemiluminescent moiety.

60. The method of claim 57, wherein one or more of the oligonucleotides represents a known gene, mutant or truncated form of a gene.

61. The method of claim 20, wherein step b) is performed by subtractive hybridization, differential display or representational difference analysis.

62. The method of claim 20, wherein the plurality of genes comprises all of a genome or a transcriptome.

63. The method of claim 20, wherein any of steps a) to e) are controlled by a program comprising computer readable instructions for directing a processor to carry out any of steps a) to d).

64. The method of claim 20, wherein any of steps a) to d) are performed by a system comprising:
a processor element; and
a computer program comprising computer readable instructions that direct the processor to perform any of steps a) to d).

65. The method of claim 32, further comprising introducing the each expression construct into a cell to produce a collection of cells, wherein each cell is a responder cell that comprises the expression construct.

66. A collection of cells produced by the method of claim 65.

67. A collection of cells, wherein each cell comprises a nucleic acid encoding a robust responder regulatory region operatively linked to a nucleic acid encoding a reporter gene.

68. The collection of claim 71, wherein robust responder regulatory regions are obtained from genes whose expression is increased or decreased at least 3-fold in the presence of perturbation compared to the absence of the perturbation.

69. The collection of claim 72, wherein genes whose expression is decreased the decrease in expression is at least 6-fold.

70. The collection of claim 71, wherein the regulatory region comprises a promoter, a silencer or an enhancer.

71. The collection of responder cells of claim 71 that comprises an addressable array.

72. A collection of responder cells, comprising a plurality of sets of cells, wherein each set is in an addressable location and the cells of each set comprise a different promoter operably linked to a reporter nucleic acid.

73. The collection of claim 72, wherein the collection comprises at least 300 sets of cells.

74. The collection of claim 72, wherein the collection comprises at least 1000 sets of cells.

75. The collection of claim 72, wherein the collection comprises at least 10,000 sets of cells.

76. The collection of claim 72, wherein the different promoters are each robust responders to a particular peturbation of interest.

77. The collection of claim 5, wherein the peturbation is exposure to a substance or a change in extracellular or intracellular condition.

78. The collection of claim 72, wherein the perturbation comprises exposure to a drug, a hormone, an extract, a protein, a nucleic acid, a lipid, a carbohydrate or a fat.

79. The collection of claim 72, wherein the perturbation increased or decreased temperature, exposure to ultraviolet light, a change in pH, a change in a salt or ion concentration, exposure to or a decrease in oxygen.

80. A method of characterizing a perturbation, the method comprising:
exposition a collection of responder cells of claim 72 with the substance to obtain a response profile for the substance; and
comparing the response profile for the substance with a response profile obtained by contacting the collection of responder cells with a characterized substance to thereby characterize the perturbation.

81. The method of claim 80, wherein the response profile for the perturbation is stored in a database.

82. The method of claim 80, wherein the perturbation comprises exposure to a drug, a hormone, an extract, a protein, a nucleic acid, a lipid, a carbohydrate or a fat.

83. The method of claim 80, wherein the perturbation increased or decreased temperature, exposure to ultraviolet light, a change in pH, a change in a salt or ion concentration, exposure to or a decrease in oxygen.

84. A database that comprises response profiles for a plurality of peturbations, wherein the response profiles are obtained by subjecting a collection of responder cells to each peturbation to obtain a response profile for the peturbations.

85. The database of claim 84, wherein the peturbations are exposure to a substance.

86. A system for identifying a regulatory region of a robust responder gene among a plurality of genes comprising:
a processor element; and
a computer program comprising computer readable instructions that direct the processor to:
determine expression of a plurality of genes in a cell in the presence of a perturbation compared to in the absence of the perturbation;
identify at least one gene whose expression is increased or decreased at least 3-fold or at least 6-fold; and
select the regulatory region of the gene that confers increased or decreased expression in response to the perturbation.

87. The system of claim 79, wherein the decrease in expression is at least 6-fold.

88. A method, comprising:
exposing each member of an addressable collection of responder cells to a known perturbation; and
determining the profile of changes in cellular reporter activity affected by perturbations.

89. The method of claim 88, further comprising:
storing the patterns in a computer readable medium to create a database, wherein each profile is identified by the perturbation giving rise to the profile.

90. The method of claim 88, further comprising:
treating the addressable collection with a test perturbation;
comparing the resulting profile to the known profiles; and
identifying profiles that are similar or that match to thereby determine targets of the test perturbation or the activity of the test perturbation.

91. A database produced by the method of claim 89.

92. The database of claim 91 that is a relational database.

93. A method for producing a collection of reporter cells comprising:
(a) identifying a plurality of protein coding sequences from a database of DNA sequences of an organism;
(b) designing primers for amplifying untranslated sequences upstream of the protein coding sequences from genomic DNA of the organism, wherein the untranslated sequences each comprise a promoter;
(c) amplifying the untranslated sequences using the primers, thereby obtaining a plurality of promoters;
(d) producing a plurality of reporter constructs, each of the reporter constructs comprising a promoter operably linked to a DNA sequence encoding a detectable marker;
(e) introducing the plurality of reporter constructs into cells to produce a plurality of reporter cells, each reporter cell comprising one of the reporter constructs to thereby produce a collection of cells.

94. The method of claim 93, wherein the collection is addressable.

95. The method of claim 94, wherein the addressable collection comprises an array.

96. The method of claim 88, wherein the array contains at least 300 reporter cells, each reporter cell comprising a different promoter.

97. An addressable array produced by the method of claim 88.

98. A method of determining the effect of a molecule on a cell comprising:
(a) providing a plurality of reporter cells, each reporter cell comprising a reporter construct that comprises a promoter that is expressible in the reporter cell;
(b) contacting the plurality of reporter cells with the molecule; and
(c) determining levels of promoter activity in each of the plurality of reporter cells.

99. The method of claim 98, wherein the reporter construct comprises a promoter operably linked to a gene encoding a marker, the method comprising determining levels of promoter activity in each of the plurality of reporter cells by determining levels of the marker in of the plurality of reporter cells.

100. The method of claim 98, wherein the plurality of reporter cells is a two dimensional array comprising at least 96 reporter cells, each of the reporter cells comprising a different promoter.

101. An isolated nucleic acid molecule, comprising a sequence of nucleotides set forth-in any of SEQ ID Nos. 1-12.

102. A collection of nucleic acid molecules, comprising the nucleic acid molecules of claim 101.

103. An isolated nucleic acid molecule of claim 101, further comprising a nucleic acid molecule encoding a reporter molecule.

104. A collection of nucleic acid molecules, comprising nucleic acid molecules of claim 103.

105. A vector, comprising a nucleic acid molecule of claim 10

106. A vector, comprising a nucleic acid molecule of claim 103.

107. A collection of vectors, comprising nucleic acid molecules of claim 104.

108. A cell, comprising a nucleic acid molecule of claim 101.

109. A collection of cells, each cell comprising a nucleic acid molecule of claim 101.

110. A collection of cells, each cell comprising a vector of claim 105.

111. The collection of cells of claim 110 that comprises an addressable array.

112. A collection of cells comprising regulatory regions from genes involved in osteogenic/osteoporotic regulation.

113. A method for generating a signature for a compound, comprising:
a) providing an addressable collection of responder cells;
b) exposing the cells to a characterized perturbation;
c) identifying cells in the collection that exhibit an altered phenotype responsive to the exposing;
d) recording the identity of the identified cells.

114. The method of claim 113, wherein the perturbation is a known modulator of a cellular activity.

115. The method of claim 113, wherein the perturbation is a compound.

116. The method of claim 113, wherein:
the altered phenotype is exhibited as the generation of electromagnetic radiation by the cell;
the identities of the identified cells are recorded as an image obtained by scanning the collection after step b), wherein the image represent a signature for the compound.

117. The method of claim 113, wherein:
the identities of the identified cells are recorded in a database.

118. A database produced by the method of claim 117.

119. The method of claim 116, further comprising storing the recorded images in a database.

120. A database produced by the method of claim 119.

121. A method, comprising:
selecting the cells in claim 113 that exhibit the altered phenotype and preparing a sub-collection.

122. The method of claim 118, further comprising treating the sub-collection with test perturbations to identify perturbations that alter the phenotype of one or more of the cells in the sub-collection.

123. The method of claim 119, wherein the perturbation is a compound.

124. A method for identifying the targets of a test perturbation, comprising:
exposing an addressable collection of responder cells to the perturbation;
identifying the cells that exhibit an altered phenotype responsive to the the exposing; and
comparing the response to a database of claim 118.

125. A method for identifying the targets of a test perturbation, comprising:
exposing an addressable collection of responder cells to the perturbation, wherein the responder cells that exhibit a response emit electromagnetic radiation;
imaging the collection; and
comparing the response to a database of claim 120.


SEQUENCE LISTING (56 sequences)

SEQ ID NO: 1 - DNA, 2011 bp, homo sapien
tattgtgatctaatatgaaccaaaagcagataatgaatagcactaggaagaacacagggatattttagttctaacaccctcctgtctccctagcccttacctccctgcacattccaaataatcttttgtaattcactgtctccgcccaccccatttactttatgccactcctagttactgtcacactaggaagaagtctaacatgcagatttagagtggcatggataaatggcaaaaaaatgcctagaaaattggtctgttcgcctttataattttggttgaaaaatactccatcgctcccaactgatgaaaacaggaagctctattcataaatataaaattcactgcctatgatatataatcatcctaataagaaaatgagttctatacatacttgtccaaaggggcaaaaaaggagatagtttcccaaagatgtttccaattttcttctgaatcagaattagcaaatcgagacgactaacatactctgtctgtgggcattattccttactacacacagcattttgtaatttatttcaaagcttccattagaaacaaaaaaatacatagcttctgttaacccactctattctaagctcatagaatcaaatactgaacaatctacattataacataagcattttactttatagaagatctgctatcagaaactctattaatgtctaaactacttaaagaactatataaactgaatacacttcaatgaaagacaaaaaatattacaatcataaagaaaactaagtattcatccaataaactatattacaatccctgtcattcatttttttaagatcttcaaactaggcatgagataatggtatacatgaaacattacatttaatctttattgtaaaggccgccatctaatagattgataataaactagacagacgtgatttaaaatttgtaaaagaatgcccagactaacactttcatgacagccaattatagtcaagcctagcaagcagtttgcaaccagaccttaaggtaaactttttttttttttacaatgagttacagattcacaagtttaagaagacaagaaaaaggaaaacagaaggaatccagccacccagcaaatatgaagcagaccccagaatgtgatacagtccaaagatgtgaattattgtatatcatcactgttgttcagaatttcacacagactcttgagccaattttgttcatttttccacagacacaataatgaactaaaaagaggaggcaaaaaggcagaggttgagcggggagtagaaaggaaagcccttaactgcagagctctgctctacaaatgcttaaccttacaggagtttgggctccttcagcatttgtattctatccaaatcctcatgagtcacaaaaattaaaaagctatatccttctggatgccaggaaaggccttaccacaagccttttgtgagagaaagagagagagagaaagagcaagggggaaaagccacagtggtaggcagtcccactttacttaagagtactgtgaggtcacaaaccacatgattctgcctctccagtaatagtgcttgcaaaaaaaaggagttttaaagcttttgcttttttggattgtgtgaatgcttcattcgcctcacaaacaaccacagaaccacaagtgcggtgcaaactttctccaggaggacagcaagaagtctctggtttttaaatggttaatctccgcaggtcactaccagccaccgagaccaacagagtcagtgagtgctctctaaccacagtctatgcagtaatagtaggtccttcaaatatttgctcattctctttttgttttgtttctttgcttttcacatgttaccagctacataatttcttgacagaaaaaaataaatataaagtctatgtactccaggcatactgtaaaactaaaacaaggtttgggtatggtttgtattttcagtttaaggctgcaagcagtatttacaacagagggtacaagttctatctgaaaaaaaaaggagggactatg

SEQ ID NO: 2 - DNA, 2041 bp, homo sapien
ggcttatagagaacttattacggtgcttgacacagtaaatctcaaaaaatgcattattattattatggttcagaggtaaagtgacttgcccaaggtcacatagctggaaaatggcagagccgggatggaaatccaggacttcgtgactgcaaagcagatgttcattggttagtgaactttagaacttcaacttttctgtaaaggaagttaattatctccatctcacagtctcatttattagataagcatataaaatgcctggcacatagtaggccctttaaatacagcttattgggccgggcgccatggctcatgcccgtaatcctagcactttgggaggccaggtgggcagatcacttgagtcagaagttcgaaaccagcctggtcaacgtagtgaaaccccatctctactaaaaatacaaaaaatttagccaggcgtggtggcgcacgcctataataccagctactcgggaggctgaggcaggagaattgcttgaacccgggaggcagatgttgcagtgagccgagatcacgccactgcactccagcctgggtgacagagtgatactacaccccccaaaaataaaataaaataaataaatacaactttttgagttgttagcaggtttttcccaaatagggctttgaagaaggtgaatatagaccctgcccgatgccggctggctaggaagaaaggagtgagggaggctgctggtgtgggaggcttgggagggaggcttggcataagtgtgataattggggctggagatttggctgcatggagcagggctggagaactgaaagggctcctatagattattttcccccatatcctgccccaatttgcagttgaagaatcctaagctgacaaaggggaaggcatttactccaggttacactgcagcttagagcccaataacctggtttggtgattccaagttagaatcatggtcttttggcagggtctcgctctgttgcccaggctggagtgcagtgacataatcatggctcactgtatccttgaccttctttctgggctcaagcaatcctcccacctcggcctcccaaagtgctaagattacaggaatgagccaccatacctggccctgaatcttgggtcttggccttagtaattaaaaccaatcaccaccatccgttgcggacttacaacctacagtgttctaaacattttatatgtttgatctcatttaatcctcacatcaatttagggacaaagagccccccaccccccgtttttttttttacagctgaggaaacacttcaaagtggtaagacatttgcccgaggtcctgaaggaagagagtaaagccatgtctgctgttttctagaggctgctactgtcccctttactgccctgaagattcagcctgcggaagacagggggttgccccagtggaattccccagccttgcctagcagagcccattccttccgcccccagatgaagcagggagaggaagctgagtcaaagaaggctgtcagggagggaaaaagaggacagagcctggagtgtggggaggggtttggggaggatatctgacctgggagggggtgttgcaaaaggccaaggatgggccagggggatcattagtttcagaaagaagtctcagggagtcttccatcactttcccttggctgaccactggaggctttcagaccaagggatgggggatccctccagcttcatccccctccctccctttcatacagttcccacaagctctgcagtttgcaaaaccctacccctcccctgagggcctgcggtttcctgcgggtctggggtcttgcctgacttggcagtggagactgcgggcagtggagagaggaggaggtggtgtaagccctttctcatgctggtgctgccacacacacacacacacacacacacacacacacacacacacacacaccctgacccctgagtcagcacttgcctgtcaaggaggggtggggtcacaggagcgcctccttaaagcccccacaacagcagctgcagtcagacacctctgccctcaccatg

SEQ ID NO: 3 - DNA, 2049 bp, homo sapien
aaaataggttaggcaactagtctgaggtcacagagctaggaaaaattggagttggggctcaaatctaggttacaaaggccagtatcttaggtattcccctagaataatcataactataggaaatatttcctatgggccaggcattgtgctgagttattttacatgcattactttatttaatgctcataattagtgattaccatcatttatataattgttttttaaacgctcccatttgctttctcttacgtttctgcaatatcagtgtgtttttatcttatagatgaggctcagggagacgtaaacctttcccagggttaacactgaaggactcagttattgattagttttctccaaggtctgacacccacatattggcatcattttatgttctgagaaaaacaccttcaaataatatcctagacaaacattactctaacaaaaacaataatactgctatttatattgtgtttcactactaacacttggattgacttgagtcccatggcaagtctaagtgttgatatctcaggttgcagatgtcaaaactacgattcaaaatacaaggagtgatttggagtcatacaattttgtccacactcactgagctacatttattcactagttcacttaagaaaccagcatgctgttacattctggcccttgagggacaaagctgaatgacaccccgtcttctgtaatttgcaggatggaacagtctgtggatccactttgaactcgtggtggaaggatgtcccttggaaggggcagatgctctgatcctggtaagccatccttgctccccaggggtcccctctcctgattcttcaccttccttcccttgaatctggtgaaaggcagtatttgcccttctctggagacatataacttgaacacttggccctgatggggaagcagctctgcagggactttttcagccatctgtaaacaatttcagtggcaacccgcgaactgtaatccatgaatgggaccacactttacaagtcatcaagtctaacttctagaccagggaattgatgggggagacagcgaaccctagagcaaagtgccaaacttctgtcgatagcttgaggctagtggaaagacctcgaggaggctactccagaagttcagcgcgtaggaagctccgataccaatagccctttgatgatggtggggttggtgaagggaacagtgctccgcaaggttatccctgccccaggcagtccaattttcactctgcagattctctctggctctaactaccccagataacaaggagtgaatgcagaatagcacgggctttagggccaatcagacattagttagaaaaattcctactacatggtttatgtaaacttgaagatgaatgattgcgaactccccgaaaagggctcagacaatgccatgcataaagaggggccctgtaatttgaggtttcagaacccgaagtgaaggggtcaggcagccgggtacggcggaaactcacagctttcgcccagcgagaggacaaaggtctgggacacactccaactgcgtccggatcttggctggatcggactctcagggtggaggagacacaagcacagcagctgcccagcgtgtgcccagccctcccaccgctggtcccggctgccaggaggctggccgctggcgggaaggggccgggaaacctcagagccccgcggagacagcagccgccttgttcctcagcccggtggcttttttttcccctgctctcccaggggccagacaccaccgccccacccctcacgccccacctccctgggggatcctttccgccccagccctgaaagcgttaatcctggagctttctgcacaccccccgaccgctcccgcccaagcttcctaaaaaagaaaggtgcaaagtttggtccaggatagaaaaatgactgatcaaaggcaggcgatacttcctgttgccgggacgctatatataacgtgatgagcgcacgggctgcggagacgcaccggagcgctcgcccagccgccgcctccaagcccctgaggtttccggggaccacaatg

SEQ ID NO: 4 - DNA, 2443 bp, homo sapien
agtaaagtatttattctagatggccatatccctacctaagacttggagttttctatgactggggaagaacggaagacaagatattgggaaagactagcagcctctactaaaagggtgatctgtgttgatgtgcgtgtgtgtgtgatgtttgtatgagcatgtgtgttatgtgttgtgtgttggtggggcagattcttgcgagcactttggtctcagatggacctgctaccagttctctctgcagacccccataggtttctcctaaacctggcctctcctattaggcagccttactcagcggcagcttctcagctccatgttttcaaggaaccacaatttatttccagcatccactgaagcatattatcagtggtgatagagggggcttgtaaaactgtttttccacttaggtattagagggtggccattacttgagagtgactatgaccacagttaatctggtaataaattctcttgggtaggaggaaaggaaaggatgctttaaggaagcatcttgccgggagacacaaagctaacaagagtggagcctgcagctggagccgcagagcctaatcactacacccgcccatctctgctagggtttcatgacttcgtatcggggattagcagtatttaactctgttgcacaaacatttggtgtattattcaggtaacaagtagctaatagaggaagttttacttttttaagacataaatttgccttttcccaaattacttggtacatagtacttttcatgtttgaagttgagatgtgggtacaataccatagctttattccagagcagggtatttgtttccaaatgccatgttcccagcagctgcccttgactgggaattggggtgtgatttgggcttttccttaaatccttgaggagctggaggggtgggtggctcgcactcctgctttctggatctgaatcctgactctgtcatggacctgtttgactttgggcaagttgactcctattcctgagccccatatttttctcttctgtaaaattcagattaaaaaaacatggctttgatcaaacattataaataatatatagacagactgcttgtttttattgtattgccagaaatgaatcctactaatattgccatctatggacagaaaatgtattacctgtcttcatcaagacccagacgaggaagaacacgaaaagcggagattaattttactgccatctccagaaccgtcatcctaatatttacttacattttattattatttcaggctcatgcacatatacttagcatggatcattggccacagactcgcatacatttaactttattaccttttgcctcatgtatctcattaaaattttgctgcttaatcaaggatctgcatattattttaattttagaattcacagttccaagactttgaaagtttcaagcgttctgggtgaatgtgttatgctctctcccgccaccatgtctttataccccctgatttctcagccactatggcaaccactttctactcttagtagcccatatttagtccaatccccagctcaggagacacttcttccagggagccccctgtgccttccagtagtatcttgtacctgccctttttgcaaagctctttcctcctggcttagaatggcccattgacctgtttgtttctcctattaaactgtaagccactcgagggtagagagcatctgttgttcaccattgcatcctcggtgctgagcactgcgtctgacatattatttagaaggtcagtaagtgctagtgggattcaggctcccagtgggtgggagagaaaggacgtaaggaagcaagtggtaaaggccctcacagagtatcagcaggctggtgtgagggagaaatgcagaggatgggtgagtagcataatcgctaatgatagggtaatgatagagcacatttcacaacacctttaagccctttcacgtgcatcagataatttgatcctcataaaagcctagagatagatatattacagggatgaaggtggagtattttgtggttatgtgatatgtttaaaattatgcagtgagtaaatgactgggttcaaaccagaccttaaaagtctgttatctttccctcgagcatgcaatgaagtctacatcatccctaccatgtccatttgatcacaccctggcctcacagctctgtggtctacaggatacctcatggtggttttattgaccagacaataatcctctttctaaggggatgcatttcattaatacatatgtagatcatgaattgtctttgactttgaggggatggtagccagagcagaaagcaaagctgattttcatccccgtctggtaatgtggttggtaatgtgaagatgggtgtattctgagataccggctccttgcagtgtgtggttccttctgttttcaggcccaagaagcccatcctgggaaggaaaatg

SEQ ID NO: 5 - DNA, 2023 bp, homo sapien
ccgtggtgacagtaggaacaagtggtgcctatgtccctccccattcagtttaccagctgagggtaaagacagacatctgggcttcacaggatttcagaaggcatgtctagggcaacactaaacacatggcttgacagaaatttgaaccaaagcatcgaacccagtgaacgaggcagaagggcagagagaaggcaggtagaagccacagaccagaggctgggacccagggcacagcagaaggtttagaatcagagggaaggcggtggtgcctcagtagagtccttgggccatggaactcaccccaggagcttttccaggctgcctgcagcctgcaatgtgggtgtagagtgtggctaagggagctgcctgctgggaccagctctactgctcaggacactcaaatccatctgtatgccactgtcatcaccccacacatactctctccaatcccggcaaaatcagtgctaatgtctcaccaacagattaaggcctggattgaagtacaagaaacaggatttttaactcaagttaattcaattccccagcgacccttgttaacttattcaccctcagagacgtattaatagttctgtcttatattgtatagaaatttgtgcagtgagttttctggtagctttacattttttttctcacttcagttagacatgtaatctatttaaaagtaatatgggaataagataaatcagtgtaggaataacttcctggcagaaatatttttactagtttctgagtgtaatatcagcccagcaaaagttatctgcaaatatagaagttctcatgtacatcaaagacactcaagttttttttaagaaataaatcattttatgctactgaaataactctgtgatgtgctattggcatttaaggagctaaacagactctatgggccagccaacttctactgcaagcattagacatgcacaggctttagactcaggcacaccttagaagttctggctttgctacttattagctatggtaactcgggcaggtcatttatcctctctaagcctcaacttcctcatctgtgaaatgggaataatatcagtcacatgccagggataaatccagggagaatggccagggggctgtgtcaaaggccagacacaacttccaccccaggtgaatgttgggaccaggacagtgagcaggcaaaccttgcccttgccctccttccctccacaatcttaaagctccttgaacaacccccatccccaccccctgagaatgtctgtgccctcctgctgaaagggtttggcctttcagtgttcccctccaccatgagctgtttccatgaaaagatctcaagggtgacttgaggctacggtcatcactaccacaagccttttcccatccctgcctctacctattgccctctaaataaggaagccagcgctgccaggcaaagaacttctgcccaatatgggtcctgggtggcctctcgcctctctctttccctgggcccccagccagctcccccctcccccagagatgctccctgctcacttcattcctgcctcatagttggaatgacagtggctcccagaacccctggggagtgtggagggtgatgggggtctggggaggcagccaggcccaagagcaggttaatgttacagccctggataagtgagctgggcgggttgacgtcagggcgatgatgggtggaggggagggccgggctgctgaagcaactataaagataggtcaaatcaaatatcatcaactagggacggagcaagcgggcgagctagagagcgtccccgagccatggtctctaccggccgcggctcagcctgggtccctctgctctcaacccgagtgcccgatggaggctttggtttcatgtcagcagccttcatctgccttccaaaaataagcccctgccgccatgccggagggagaaaaacaagaagggcggtatttttagggccattaattctgaccacgtgcctgagaggcaaggtggatggccctgggacagaaactgttcatcactatg

SEQ ID NO: 6 - DNA, 2423 bp, homo sapien
ctgcccagcatggtgcttggccctgggactggccacataatatctgggccaggtgcaaaattagtacggggcagggggtactttgttcataggtgattcagaaccacatatggtgacctcagagtaggaaaccaagtgtggggcccttaagagctggggggccctgtacgactgtccaggttgcaggccccacagctcgcctcctgatatcctgtgctccatgcttgtctgttgaaggaaggagtgaatggatgaagagcaggtggtgggggtggtttgagggccttgcctggtgggtgggtagaggcccctccctggcatggggctcaagacctgttccatcccacagcctggggcctgtgtgtaaatggccaggacctgcaggctggcatttttctgctccttgcctggcctctggcctcccctttctccacccatgtggcccctcaggctgccatctagtccaaaagtccccaagggagacccagagggccacttggccaaactacttctgctccagaaaactgtagaagaccataattctcttccccagctctcctgctccaggaaggacagccccaaagtgaggcttagccagagcccctcccagacaagcgcccccgcttccccaacctcagcccttcccagttcatcccaaaggccctctggggacccactctctcacccagccccaggaggggaaggagacaggatgaacttttaccccgctgccctcactgccactctgggtgcagtaattcccttgagatcccacaccggcagagggaccggtgggttctgagtggtctggggactccctgtgacagcgtgcatggctcggtattgattgagggatgaatggatgaggagagacaggagaggaggccgatggggaggtctcaggcacagacccttggaggggaagaggatgtgaagaccagcggctggctccccaggcactgccacgaggagggctgatgggaagccctagtggtggggctggggtgtctggtctcaggctgaggggtggctggaaagatacagggccccgaagaggaggaggtgggaagaacccccccagctcacacgcagttcacttattcactcaacaaatcgtgactgcgcagctacagtggctaccaggcgctgggttcaaggcactgcgggtaccagaggtgcggagaagatcgctgatccgggccccagtgctctgggtgtctagcgggggtaagaaggcaataaagaaggcacggagtaactcaaacagcaattccagacagcaagagaaactacaggaaagaaaacaaacgtgcgaggggcgaggcgaggaaacaacctcagcttggcaggtcttggaggtctctgggaggagaaagcagcgtctgatgggggcgggaggtggtgagtggggagaggtccaggcggagggaatggcgagcgcagagacaggctggcaacggcttcagggaggcgcggaggggtcagcgtggctggcttaaaaggatacagggactgaggggcaagaccggctcaagggtcaccgcttccaggaagccttctatttccgcgccacctccgcgctcccccaacttttcccaccgcggtccgcagcccacccgtcctgctcgggccgccttcctggtccggaccgcgagtgccgagagggcagggccggctccgattcctccagccgcatccccgcgacgtcccgccaggctctaggcaccccgtgggcactcagtaaacatttgtcgagcgctctagagggaatgaatgaacccactgggcacagctggggggagggcggggccgagggcaggtgggaggccgccggcgcgggaggggcccctcgaagcccgtcctcctcctcctcctcctccgcccaggccccagcgcgtaccactctggcgctcccgaggcggcctcttgtgcgatccagggcgcacaaggctgggagagcgccccggggcccctgctaaccgcgccggaggttggaagagggtgggttgccgccgcccgagggcgagagcgccagaggagcgggaagaaggagcgctcgcccgcccgcctgcctcctcgctgcctccccggcgttggctctctggactcctaggcttgctggctgctcctcccacccgcgcccgcctcctcactcgccttttcgttcgccggggctgctttccaagccctgcggtgcgcccgggcgagtgcggggcgaggggcccggggccagcaccgagcagggggcgggggtccgggcagagcgcggccggccggggaggggccatgtctggcgcgggcgcagcggggcccgtctgcagcaagtgaccgagcggcgcggacggccgcctgccccctctgccacctggggcggtgcgggcccggagcccggagcccgggtagcgcgtagagccggcgcgatg

SEQ ID NO: 7 - DNA, 2363 bp, homo sapien
gttgacatctgtgtgtgtgtgaagataaatgggtgcctgtttggatgcaggacatgatacagggcattgctggtatgctgtcagaaacctcatgtgaaaacgaaccacccgaaggacggcttctggcccttggagtcactcactcacttgtgggactgttcagggtataatctgtctccagtctacaattgtcgttttactatgggaatagaaagtttgaatcaaaattgaacattgaatcaaaatcaaaactattaaacaaatagacaattaacaactactaaacaaaatatggttctttctatggtaatttaaaaaatggctgtaacattgtacattttaggaggaaaaagaatcaaaagatgactagaaacctaagtgagcctggagaaaaagttaagtggagacattgtagctaaacgatgagcatgaatataggaaaatttaacctagaaactgagaaaggattccagtgaaccaaatatcttgacacagcccttggaacacagcaccaggacgcgtgagtaatggtgtgcacgtcagaaagataccagaactaccacctcagtgggaaaaacatcccctgggcttgtccgcagggcctctctggctgcaccccggctgctactgtcactagttagaatggaaaatgtgatgaacctgatttgtctttcctaatctggacacacaatcgattctaccatttttattttcaggaccaaggcatttggcgttttttgtgtgcctagtaatgttgtttgccgagtgtattagtcagggttctctagagggacagaactaataggggatggagatatatttctgagtttattaagtattaactcacacgatcacaaggtcccacaataggctgtctgcaagctaaggatcgaggagagccagtccaagttccccgactgaagaacttggagtcccatattcaaggacaggaagcatccagcatgggagaaagataggctgaaagtctaggccagtctcgtcttttcacgtttttctgcctgctttatattctaaccgtgctggcggctgattagatggtgcctagctagattaagggtgggtctacctttcccagcccactgattcaaatgttaatctcctttggcaacaccctcacagacacacccgggatcaatactttgcatcctgcaatccaatcaagttgacagtaagtattaaccatcacaccaagcttttgctggagcctcttgatgacaattttgattgagtcagaaggatgaatttcgcagagatgttggttatattaacaactcattgcacagatggaggacctgaggtccacatccagctacaaatttctgcctgcctcctgcctccaggctgatctggggacgtggtggcctctcagcattattgcccatgccctagtctggtagaagagtggtttaaaagtgtgactgttttattcttcataagaatcaggctgccttggttgaaattgtggccccatcactttgcaactttgtggcctctggcaagctatggcacttcactgacccatatatgtgatggagataatgatacggttattacaggagcacacttgatgataggtgtaaagcactcagtacaatgcctgtttgtaggaagcatctaataaattctagttgccagtataactaagcacttgccctatttttcaaatgctattttagccagatcaaataggtaggaaaaagcctgtcaatcatgaagtttatactttcctgtttctaaaaaggtacacttctaaaaatttatataattcatttatagctattaacttaaacttggaaagtttggatatttggtctgtcttcacaagtgtttatctgagccctacctctcaaattaacatgtatcaccattgatgtgcattatgttgattcttatacctattatatgcatgtgtgaaactaagccccataaaaacagaatttaggcattcctgctgaaaggaagtgaattgaagggaagagaagcagagcctttgcaaagagaaaattgtcctatctctcaaccagtgtcagaatgtggaaatgtttacaaaatgctcattaaaagaaatagggattgcaagatagaaacaaattctggtgcacaagtttacactagggagaaagaaaggctaggcccctataggggattttgttatccaattactgcaacctgacttttagggggagaggaagagtggtagggggagggagagagagaggaagagtttccaaacttgtctccagtgacaggagacatttacgttccacaagataaaactgccacttagagcccagggaagctaaaccttcctggcttggcctaggagctcgagcggagtcagt

SEQ ID NO: 8 - DNA, 2203 bp, homo sapien
aatccatctattttactctttataagaaatcttttaaatgaaaataaagataggttgaaagttaaacaaaatcagaaaaaacataccatacagagtaagcatatgaaaactgctgtggcaatgttaataaaaaataaagtagactttaggacaaaaagtgatatctgagattaagtggagatcttcacagttatcaaaatattaatttataagatataaaaatctaaagattcaaaatattctaaatatgtatgtgcctcataacagtgcttcaaagaacaggaagaaatactgaaaaaaatgaaagaaaggtaggaatccataatcgcagattggaaaaatccacatttatttgtttgccaagagagaccatgcactgagccataagttaaatttcaataaacttctaaagtttgacatcttagagagtatgttctcagatcataaacatccagtgtagaaatcaaaaatataatatttaataaagctcaaatatttggaaattaacaaaaaataaatcacaagagaaattagaaattatgttaaataaatgacaatgaacataaagcattcctgaattcatgagaaacagctaaagaactgctagaaggaaatctatatttaaaagtttatatgataaaagaagaaaggtgtaaaatcataatttaactttccaaattgataggtagaaaaagaaaatgaaatttaaaaccaaaacaggtcaaatgaataatataataaatagaacagaatcaataaaaacacaaaaaataaaaaggcagaagtttttttggaaaagattaggaaaattgataaacccctaacataagtgatcaataaaaggagaaaagcacaacttaatcattttaaaaattacacaggggatatctatatagatgctatagacttcaagaagataataaggcaatttttaaaactgccaattgccaatgatttgacaatttagatgaattgaacaaattacttgaaaaatacaatatatcaaaaattgaccctccctaaagatattaatacaaaacctatctaaccctatgtctaataaaaaatagccaatacaatgcacgaagaaaactagagactcagatagtttcactaggaaattttatcaagcattttaaagagaattaattttaatctgaagttactttagaaaacagaagaggaagtgcatttccccgatcatttgttgatgccagtataccccaataaaaaacctgacaaaaacattataagaaaataaaattatagaccaatatattttatgagaggatgtcaaaattcttaaccaaacattagtcaattgaatcatccaatatataaaaatgataatatatcataaccaaatggagattaattcacaaatgcaaagctgccttcatattttaaaattcaatttgcataaattgtccccgttaacagaataaaggagaaaatccttatgttcatttcagtaggtttcgaaaagcatatgacaaaatgcaaaaccattttgttataaaaactctctgcaacttaggaatagtaggggacctactgaatctgataaagggtgtccataaaaaaatatgcagttcacatcatactccatagtgaaatattaggtttccctttaaaattcagaacaaagtgaagatgtcagctctcgccatttttagttaaccttggcataaagattgcaaaggaagaagtaagcctgaatgtacttgcaggtaaaatgattgtttatgtgtacgtttctaaagcatgtagtttaaaactactagaattaataaagaaattaagcatggtgggtgctcccgaatcgatgaggaaagccgctctccccggcagatcctcccggccggggcgcctccatcaccctgcctgcgcctcggcacgctggcaaggagcccgggaagagacgccgggagcgacttatgaaaatatgcatcagtttaatactgtcttggaattcatgagatggaagcataggtcaaagctgtttggagaaaatcggaagtacagttttatctagccacatcttggaggagtcgtaagaaagcagtgggagttgaagtcattgtcaagtgcttgcgatcttttacaagaaaatctcactgaatgacagtcatttaaattggtgaagtagcaagaccaattactaaaggtgacagtacacaggaaacattacaattgaacaagt

SEQ ID NO: 9 - DNA, 2402 bp, homo sapien
gtatttaccatgcacctactatagcaggcaacatttttaggaaatggtgaatgttacagaggtgaataatacagcaagagtcgttgaacatatggagtttatctattagttggggagtgaatgttgacaaaggaataagtaaatacataggcaagaaagatacattacctgtgaaacagcagcaggtagactgacagtggagtatctaatacagcctatggaagccagaagatagtgggatgacatttttggagtactagtagaaatgtcatatgaagaactctgtaggaatgtaacatacggtcccatatatgaagctcctgggtcaagtatacctgaacataattcagggatttgagggactttcttgtaacctgaggatcaagatgtcaaggaattaaaaacatgtataaaacattgttgtataaaaacccattaaaaagaatggaagacactatagtaaaatcattgtgggtttagttgttataacacattttaaaaatctttgatcccaatcaatatttataagaaagaagaaatatggaattatttcctgagtcaaggagcagggagagaatgaggaagaagaggaggaggaggagggggaggaggagacaataaacctacttcccaaagttaacaaacaaaaagtgggaagaggtcaaagactacaaggagtagaattaacgtcaattgtttctatgtttgagtctgaaaattttttgtcccttctccaccaacctatatattgatacacatataaatgctaaaggcatttttgaatttgaacagatcattttctttgtatggctgcctttaaaaaaaattcaacctggtcactcttcctcaacatttactgaggtctaagtgttcaatttagaacacatgctttaataactcagagacctgtcatttgtcacaaatcttgcctagagaaatactcattagcgaattaggcagaaagaggatgcaaaataaaaaggcacagtagtcccctgatatccatggaagactggttccaggacaccaccaaacccctccccgcaaataccaaaatccatggatgttcaagtttcttaacatatcatggcatagtatttgcatttaacctacacacatcctcttgtacacttgaaattatctttagattatttataatacttaatagaatgtaaatgctatgtaactagttgtgtatcatttaggaaatgatcacaagaaaaaaagtctacagatgttagtccagacacagccatcctttttttttttttcaaatatttttgatctgtggttcattgcatccacagatgtggaacccatggatactgtgggctaactgtattaataaaaaagtggaaacatcctaagtttcatgggtgtttaaattggtcagcaacttccttctgaagaagtatcagaatttgtgagcaatgttaatatttttgttttctcactaagagccacagttctgaatagaggtttttaaaaagccctagcaaggtttctttagcaatgaaactaacatttaactgtatcatcagcttcgtgttacatctctttcctgactgttgggtgagccctcctcggatgcttgcttctggctacacgcccctttacccttttctctgcactgttttcatctttataaagtcagagttggtgtctataggctctctactgccacattcaagacctgcctcgctcaatgtcaccttcaagatgcagaaatagggatttgggaaggggattgtgaaattttcgaagtcttccaaaatactttgagaaactatatttggaagactttggggggagaggttggacaggaagggtcttcagagatcatcaaatttaactttctaaatcctaaggaggaaaccgagactccaggatgtgaagtcccttctctaccaaactagaatggatgcaggaggaatgtctgaggtgcaatccttatcctttagcaaaggtgtcctctgcgtcttctttaacccatctcttggacctccagaaagacagctgaggatggcaaggggagtctggaaccactggagtagcccccagcctcctccttggagggcccccatgaaggaggcccttcagtgacagagattgagagagagggagggcgaaaggaaggaaggggagccagaggtgggagtggaagaggcagcctcgcctggggctgattggctcccgaggccagggctctccaagcggtttataagagttggggctgccgggcgccctgcccgctcgcccgcgcgccccaggagccaaagccgggctccaagtcggcgccccacgtcgaggctccgccgcagcctccggagttggccgcagacaagaaggggagggagcgggagagggaggagagctccgaagcgagagggccgagcgccatg

SEQ ID NO: 10 - DNA, 2499 bp, homo sapien
agatgaggaaactgaggtccagacagccgaagagtggtagtgtccaggacacacaactggtaagcgggcaagcacaggctgttgcttagcccagactcatttcccagggcctcatgcattcgcttcctccgcgatccttaaagccctgcgctccaggcatccccagcccctccctctgcctcagtttccccacttggtaccgggaggtggtaggtttggggtcgaagggcccctcctcttagagctccagcgtgccctccccagccaaacacagaaatcccgccccgttcagccccaacccccgcggactcctccttgccttcccctaagtcgagggtcccaggcggcccggtccgagccggccgatagcttttgggagtgggggtgggaacgggggagggaggtgaagcctgagagtgggtgtctggattgagccccaggtctggcagcctcgagcctccggggttggggctgggcaagctggagaggcccggccagcagctgaatgggtcgagactcggagacccggacccgaagagacgctgggcagggagggagcgggatgtgtggctgcagacctgggcgggggtcggggctggcctagggccgagaggaacgacaggcctgggatgggactgagggcaggggacgaggcgagggtggggctggacgtgggggagggcggcagcagccaagccgggctcggggctggcagccgagcggcctccccagggaccccgacccggcccgaacgggagcccagtggactgacagcgtcgcggccgggggcgcgcgggggtaccgggcagcctcctcaggggattcgcccatgatgaaagagggctcgcttctcggctcagggtctctattcgccagcgggggccggatgatcaagggaaaaaaaatttaaaagcccgtgctttccagaagagaatgaagcggcggcggcgtcccgggttccctgctcgggtctcgatgttacagctgcccccgccccgtctccccagcactcacatcccgccgccgtaagactccgggcctcggcctctagcgcaatgtcccggggcggggggcggaaggctcctctcggcctctccacactcccgcgtcggcggctgcggagggggtgggggcgggagaggcccgggagggcgcgggggagggaagaggcgcccggccggggagaaggggagcggcagacgccgaggcgagggatgcgcgcggcgggcggtggctccgagcggcggccgggcggggggcgctggaggccaggccggccagcggggggtatcccgagagctccatgaagtccccccggggccgcggacggggcgctggcttggggaggctgtcgggggggccccgacatccatggcaaggcgggggccgcggcggcgcgctcggagtaagtcggggctggggacccgcgccgaggggaagtggccggagtcggggaggagcgactccgggcctggccggagcagccaggctgctctgtctcggtgtcagtcggcggcgcctcctcggaacccgggggagtcgccagccccgcgccgctcggctcggtggcttttttggaaacttgcaaatgttttcgtagagagaaaagggggagggagggagcgagggagtgaccgaaacggagcttggggccgctggaagaactgaggccaaggccgggggagctagagacggactgacagacaggcagaccgacagagcgtcggggccgctgcgcgcccgagcggcacaggcgcaagcggggctctggccaaggatggggaaggggtgcgggaggcggctgccgagggtctgggatctcaggaggccgaacggccgggggctggcggccggaacacctaagggctcagtgtggctgcaaagttgagatcgcaccccctaactgcacgccccgcgcggctcagaacgcgccccctgcccggccctgactccctacgccgaaagtcgcggagctaaaaataacagtcctgcgcgccccccgcagaccgcgaccccgacccctcccccgccccctccccccactgggcgtggggcgaagccacagctcccatttccccaaaagaaaaaaaaagaaagaaagaaagaaagaaagaaagaaaaggcggcgcgggaggggggcggggggcgggccgggggaggcgggcccggccatatggatgtgatttcttcgctccgaggcagacgggccgctccgcagcgctcggcgcccgcccgccgcccgcccggcctccggctctccctccctccctcctgtccctccctccctccctcctttgcgctgctcgctcgctcgctcgctcgctcgccctcagcgcatgggccccgcgccgggccccggggcctcgggccgccgggacgccggggtcccataggccggggcgtgggcggggcggccagcctgacgcagctctgcaccccctaccaccccagggccggcggcggcggctgccccgagggacgcggccctaggcggtggcg

SEQ ID NO: 11 - DNA, 2288 bp, homo sapien
atattagggtgtcgatttgagatctttgcagctttgtgatgtgtgcatttagtgctataaatttccctcttaacactgctttaactgtgtcccagagattctggtacattgtctctttgttctcattggtttccaagaacttcttgatttctgcctgaatttttttagtcctgagttctaatttgattgcattgtggtctgagagactgtttgttatgattttagttcttttgcttttgctgaggaatgttttacttccaattatgtggtcgattttagaataagttccatgtggtactgagaagaatgtatattctgttgatttgggttggagagttctgtagatgtctattaggtccacttgatacagagctgagttcaagccctgaatatccttgctaattttctgtctcattgatcctctctaatattggtagtagaatgttaaagtctcccactattattgtgtgggagtctgagtatctttgtaagtctctaagaacttattttatgaatctgggtgctcctgtatagggtgcatatatatttagagtagttagctcttgttgaactgttccctttaccatcatgcaaggccttctttgtctttttttttatcttgttggtttaaagtctgttttgtcagagactaggattgcaacccatgctttttttttttttttttttctttccatttgcttggtaaattttcctccatccctttgttttgaacctatgtgtgtctttgcacatgaaatggatctcctgaatatagcacatcaatgggtcctgactttttattcaatttgccagtctgtgtcttttaattggggcatttagcccatttacatttaaggttagcattcttatgtgtgaatttgatccatcatcatgatgctatctggttattttgcacaacagttgatgcagtttctacatagtgccattggttttatattttggtgtgtttttgcagtggctggtactggtttttcctttccatatttagtgcttctttcaggagctcttgcaaggcagaccaaatggtaacaaaatctctcagcatttgcttgcccagaaatgattttatttcttcttcgcttatgaagcttagtttggctgaatattaaattctgggttgaaaattcttttctttaagaatgttgaatattggcctccaatctcttctagcttgtagagtttctgttgagaggtcttctgttagtctgaagggctttgctttgtaggttactttgcctttctctctggctgcccttaatattttttcattcatttcaaccttggagaatctgatgattatgtgtcttggggttgatcttctcatgaaatatcttagtggtgttctctgtatttcctgaatttgcatgttggccagtcttgctatgttggggaagttctcctggataaaggataggtaaattctatgggtaatacagtagatatagtgcaacaggaacttaccagttaagatacagtcataaccactcacccctagttggaatgtaggtttcacacaactcccactgatgaaaagaaatatatgtatttttcaactgtttaaccctttgttaagttttcttgtgtaaaattatctgcagagccatgaaaaaccatttgatatttgtgactaagcagcctgtttggatgattatgctcttcagtatgaatggtgagctgttaaatgacatgctcaatcattgctatggaagaaatttgttcttactagcaacttgaagcttaaagaaacatttataggaaagaaaattactcaaagctttaaataaggctacttttagagttggccttagactacctagagggcatgatgattaatctttcacaaattacagattttatttgttcatgtccagtgaggtgacttcttggtggacatcttcattgcaattttcagcagctctatcaatgacacatgttaactgaagctgacatgggttgctcttgctctcttggaatgtctttatttctgtcctaatatgcaaaggtagtgccagaatttcttaataggagggcctcaggtataacaatctagttgacaggaaaagcaatggaatcttcactgcatttgcatcacaagcatactgttttttcttacgtgtgttttttagggtgtcttgggatgttgatcctctttaagtcaaatagaaaaaatgaaaatgaaatgccatagccaatattagagatatattaattttagtctttgttgcttttatatttttctaggacaaagagatcttcaaaaatcaaaa

SEQ ID NO: 12 - DNA, 2475 bp, homo sapien
gaaaaactttgaatggacctttgaaaacggtagaattgacaatggttagctgcaagtgatattttcaaggcaaacagacactctcccaaagtattaaataacccagcattctaagttgcaggtggaaggtagccattagtgaagagagagaaaaaaaaaaagaaatagctcgtctgtatttagatttatcatttctgactattgctcttccctggaaaacgggtaggtacagtcatcctgtacttcgatcccaaatcagtctctggagactacttatttatttatttatttatttatggacttctttctttcaagcgttcgaactcatttccaccacaagagggcagccatctctaaaaaaaaaaaaatagggccaaaatttatgtaagttgtgcttggaacaagcattcagtagttcctcagaaatcatacaccctacataaaagagattctgcaatgggcagcactaacatgaaacagtgttcagaagtacccattttccctcagattctaaactgacaaggtttccacttatcaggttatgaagttctaaagctgcaagacatccttgaggtcatcacaggatatttatttattttttcttcgggtgcatccaatagttatcaacttttcctcctctttaaaagctacttaaatctcattgaagttttgttttgttttgtttttgaaatctaagtaatgagagaaacaattgttaacttctcaattaaacttgataggaaaggaaataatttcagaagccctgtgtccatgagtaggatatgttttattgcctccttgtttgcggtgcaatgactctgagtgacaatcaacttctatagcaccttttttttttttttttcaggaaataaagtagcatgttcctgaataattcccccacccccttttattttcctggtagtcaggcttcctccaaaataccttatttgacctttatacctttagaaacagcaagtgcctaattcgcctctgtgggttgctaatccgatttacgtgagcggaacctagtattattttagctcccctaccgaaaaaataatacacatggataatagttctattaccagctcctgcttctgacttttttctctctgtttcgcaggcccgatagctctgggaaagcagaacttggccttttccaaaaattttctgcccttggttttggggatcatttgggcaagcccgaggtgctgtgcatgggggctcctggaatcctgggaagggcagaaagccttggccccagactcatcgtgcagcagctctgagcagtatttcggctgaggagtgacttcagtgaatattcagctgaggagtgacttggccacgtgtcacagccctacttcttgggggcctggtggaagagggtggcgtagaaggttccaaggtcccaaactggaattgtcctgtatgcttggttcacacagtgcgttattttaccttcctctgagctgctaatcgcctgcctctgagctgggtgagataaatatcacaaggcacaaagtgattgtacaataaaaaaatcaaatccctcccatccatccttcagtctgccacacacgcagtctacgttacacacatgtcacgtaaagcaggatgacatccatgtcacatacatagacatattaaccgaaatgtggcccttcggttgcatatattctcatacatgaatatatttatagaaatatatgcacatatttttgtatattggatatatttatgtaactataaatttacatgcgtatggatatgaaaataaatgcatacacatttatgtaaaaaaatttgtacacatgcatttacatatgtaaatacatacatctctatgtattaatgtttaaaaacactcaatttccagcctgctgttttcttttaattttcctcctattccggggaaacagaagcgtggatcccacgtctatgctatgccaaaatacgctgtaattgaggtgttttgttttgttttgttttttgaaatcgtatattaccgaaaaacttcaaactgaaagttgaataacgggcccagcggggaaataagaggccagaccctgaccctgcatttgtcctggatttcgcctccagagtccccgcgagggtccggcgcgccagctgatctctcctttgagagcagggagtggaggcgcgagcgccccccttggcggccgcgcgcccccgccctccgccccaccccgccgcggctgcccgggcgcgccgtccacacccctgcgcgcagctcccgcccgctcggggatccccggcgagccgcgccgcgaagggggaggtgttcggccgcggccgggagggagccggcaggcggcgtcccctttaaaagccgcgagcgccgcgccacggcgccgccgccgccgtcgccgccgccggagtcctcgccccgccgcgctgcgcccggctcgcgctgcgctagtcgctccgcttcccacaccccgccggggactggca

SEQ ID NO: 13 - DNA, 35 bp, Artificial Sequence
  primer
agtcgaattctattgtgatctaatatgaaccaaaa

SEQ ID NO: 14 - DNA, 35 bp, Artificial Sequence
  primer
agtcctcgagggcttatagagaacttattacggtg

SEQ ID NO: 15 - DNA, 35 bp, Artificial Sequence
  primer
agtcgaattcaaaataggttaggcaactagtctga

SEQ ID NO: 16 - DNA, 35 bp, Artificial Sequence
  primer
agtcaagcttagtaaagtatttattctagatggcc

SEQ ID NO: 17 - DNA, 35 bp, Artificial Sequence
  primer
agtcctcgagccgtggtgacagtaggaacaagtgg

SEQ ID NO: 18 - DNA, 30 bp, Artificial Sequence
  primer
agtcctcgagctgcccagcatggtgcttgg

SEQ ID NO: 19 - DNA, 35 bp, Artificial Sequence
  primer
agtcccgcgggttgacatctgtgtgtgtgtgaaga

SEQ ID NO: 20 - DNA, 36 bp, Artificial Sequence
  primer
aagtcctcgagaatccatctattttactctttataa

SEQ ID NO: 21 - DNA, 35 bp, Artificial Sequence
  primer
agtcctcgaggtatttaccatgcacctactatagc

SEQ ID NO: 22 - DNA, 35 bp, Artificial Sequence
  primer
agtcgaattcagatgaggaaactgaggtccagaca

SEQ ID NO: 23 - DNA, 35 bp, Artificial Sequence
  primer
agtcgaattcatattagggtgtcgatttgagatct

SEQ ID NO: 24 - DNA, 35 bp, Artificial Sequence
  primer
agtcgaattcgaaaaactttgaatggacctttgaa

SEQ ID NO: 25 - DNA, 35 bp, Artificial Sequence
  primer
agtcacgcgtagtccctcctttttttttcagatag

SEQ ID NO: 26 - DNA, 34 bp, Artificial Sequence
  primer
agtcaagcttggtgagggcagaggtgtctgactg

SEQ ID NO: 27 - DNA, 31 bp, Artificial Sequence
  primer
agtcacgcgttgtggtccccggaaacctcag

SEQ ID NO: 28 - DNA, 32 bp, Artificial Sequence
  primer
agtcacgcgttttccttcccaggatgggcttc

SEQ ID NO: 29 - DNA, 35 bp, Artificial Sequence
  primer
agtcaagcttagtgatgaacagtttctgtcccagg

SEQ ID NO: 30 - DNA, 30 bp, Artificial Sequence
  primer
agtcaagcttcgcgccggctctacgcgcta

SEQ ID NO: 31 - DNA, 32 bp, Artificial Sequence
  primer
agtcgaattcgactccgctcgagctcctaggc

SEQ ID NO: 32 - DNA, 36 bp, Artificial Sequence
  primer
aagtcaagctttgttcaattgtaatgtttcctgtgt

SEQ ID NO: 33 - DNA, 28 bp, Artificial Sequence
  primer
agtcaagcttggcgctcggccctctcgc

SEQ ID NO: 34 - DNA, 28 bp, Artificial Sequence
  primer
agtcacgcgtcgccaccgcctagggccg

SEQ ID NO: 35 - DNA, 35 bp, Artificial Sequence
  primer
agtcacgcgtttttgatttttgaagatctctttgt

SEQ ID NO: 36 - DNA, 28 bp, Artificial Sequence
  primer
agtcacgcgttgccagtccccggcgggg

SEQ ID NO: 37 - DNA, 7278 bp, Artificial Sequence
  pDEST12.2 vector (Invitrogen)
tcgcgaatgcatgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagcctaggccgcgggacggataacaatttcacacaggaaacagctatgaccattaggcctttgcaaaaagctatttaggtgacactatagaaggtacgcctgcaggtaccggatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatcgcgtgcatgcgacgtcatagctctctccctatagtgagtcgtattataagctaggcactggccgtcgttttacaacgtcgtgactgggaaaactgctagcttgggatctttgtgaaggaaccttacttctgtggtgtgacataattggacaaactacctacagagatttaaagctctaaggtaaatataaaatttttaagtgtataatgtgttaaactagctgcatatgcttgctgcttgagagttttgcttactgagtatgatttatgaaaatattatacacaggagctagtgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgatcctgcattaatgaatcggccaacgcgcggggagaggcggtttgcgtattggctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 38 - DNA, 20 bp, Artificial Sequence
  primer
gactctctagtccacgttcc

SEQ ID NO: 39 - DNA, 20 bp, Artificial Sequence
  primer
ggctgaggaaactaacaaag

SEQ ID NO: 40 - DNA, 19 bp, Artificial Sequence
  primer
gtggctttaccaacagtac

SEQ ID NO: 41 - DNA, 39 bp, Artificial Sequence
  primer
gcgcggcaacgcgtataagttggaggtctggagtggcta

SEQ ID NO: 42 - DNA, 33 bp, Artificial Sequence
  primer
gctaggaagcttgctctgttggtgcgcggagct

SEQ ID NO: 43 - DNA, 20 bp, Artificial Sequence
  primer
gcgtataagttggaggtctg

SEQ ID NO: 44 - DNA, 4987 bp, Artificial Sequence
  pNF_B-Luc vector (Clontech)
ggtaccgagctcttacgcgtgctagcgggaatttccgggaatttccgggaatttccgggaatttccagatctgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 45 - DNA, 21 bp, Artificial Sequence
  attB
ctgcttttttatactaacttg

SEQ ID NO: 46 - DNA, 34 bp, Artificial Sequence
  Lox P site
ataacttcgtataatgtatgctatacgaagttat

SEQ ID NO: 47 - DNA, 1032 bp, Escherichia coli
  CDS (1)...(1032): nucleotide sequence encoding Cre recombinase
atgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 48 - protein, 343 aa, Escherichia coli
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 49 - DNA, 1272 bp, Saccharomyces cerevisiae
  CDS (1)...(1272): nucleotide sequence encoding Flip recombinase
atgccacaatttggtatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgctgagtttcgatattgtcaataaatcactccagtttaaatacaagacgcaaaaagcaacaattctggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtaggatcgatccacttgtatatttggatgaatttttgaggaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataa

SEQ ID NO: 50 - protein, 422 aa, Saccharomyces cerevisiae
PQFGILCKTPPKVLVRQFVERFERPSGEKIALCAAELTYLCWMITHNGTAIKRATFMSYNTIISNSLSFDIVNKSLQFKYKTQKATILEASLKKLIPAWEFTIIPYYGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYSIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 51 - DNA, 66 bp, Bacteriophage mu
  CDS (1)...(66): nucleotide sequence encoding GIN recombinase
tcaactctgtataaaaaacaccccgcgaaacgagcgcatatagaaaacgacgatcgaatcaattaa

SEQ ID NO: 52 - protein, 21 aa, bacteriophage mu
STLYKKHPAKRAHIENDDRIN

SEQ ID NO: 53 - DNA, 69 bp, Bacteriophage mu
  CDS (1)...(69): nucleotide sequence encoding Gin recombinase
tataaaaaacatcccgcgaaacgaacgcatatagaaaacgacgatcgaatcaatcaaatcgatcggtaa

SEQ ID NO: 54 - protein, 22 aa, bacteriophage mu
  Gin recombinase of bacteriophage mu
YKKHPAKRTHIENDDRINQIDR

SEQ ID NO: 55 - DNA, 555 bp, Escherichia coli
  CDS (1)...(555): nucleotide sequence encoding PIN recombinase
atgcttattggctatgtacgcgtatcaacaaatgaccagaacacagatctacaacgtaatgcgctgaactgtgcaggatgcgagctgatttttgaagacaagataagcggcacaaagtccgaaaggccgggactgaaaaaactgctcaggacattatcggcaggtgacactctggttgtctggaagctggatcggctggggcgtagtatgcggcatcttgtcgtgctggtggaggagttgcgcgaacgaggcatcaactttcgtagtctgacggattcaattgataccagcacaccaatgggacgctttttctttcatgtgatgggtgccctggctgaaatggagcgtgaactgattgttgaacgaacaaaagctggactggaaactgctcgtgcacagggacgaattggtggacgtcgtcccaaacttacaccagaacaatgggcacaagctggacgattaattgcagcaggaactcctcgccagaaggtggcgattatctatgatgttggtgtgtcaactttgtataagaggtttcctgcaggggataaataa

SEQ ID NO: 56 - protein, 184 aa, Escherichia coli [sequence omitted]